SEQUENCE LISTING (141 sequences)

SEQ ID NO: 1 - DNA, 20 bp, synthetic construct
  source 1..20: mol_type = other DNA
actcctacgggaggcagcag

SEQ ID NO: 2 - DNA, 17 bp, synthetic construct
  source 1..17: mol_type = other DNA
attaccgcggctgctgg

SEQ ID NO: 3 - DNA, 15 bp, synthetic construct
  source 1..15: mol_type = other DNA
tacgggaggcagcag

SEQ ID NO: 4 - DNA, 50 bp, synthetic construct
  source 1..50: mol_type = other DNA
ccggccgaaggcctccatcccgcacgcggcgtcgctgcgtcaggggccgg

SEQ ID NO: 5 - DNA, 50 bp, synthetic construct; identical to SEQ ID NO: 4
  source 1..50: mol_type = other DNA

SEQ ID NO: 6 - DNA, 50 bp, synthetic construct
  source 1..50: mol_type = other DNA
ccggcacacgcggcatggctggatcaggcttgcgcccattgtccagccgg

SEQ ID NO: 7 - DNA, 49 bp, synthetic construct
  source 1..49: mol_type = other DNA
ccggccggataggaccacaggatgcatgtcgtgtggtggaaagcgccgg

SEQ ID NO: 8 - DNA, 49 bp, synthetic construct
  source 1..49: mol_type = other DNA
ccgggcggataggaccacgggatgcatgtgttgtggtggaaagccccgg

SEQ ID NO: 9 - DNA, 49 bp, synthetic construct
  source 1..49: mol_type = other DNA
ccggccgaataggaccacgcgcttcatggtgtgtggtggaaagcgccgg

SEQ ID NO: 10 - DNA, 180 bp, synthetic construct
  source 1..180: mol_type = other DNA
actcctacgggaggcagcagggggaatattgcacaatgggcgcaagcctgatgcagcgacgccgcgtgcgggatgacggccttcgggttgtaaaccgcttttgactgggagcaagcccttcggggtgagtgtacctttcgaataagctccggctaactacgtgccagcagccgcggtaat

SEQ ID NO: 11 - DNA, 172 bp, synthetic construct
  source 1..172: mol_type = other DNA
actcctacgggaggcagcagggggaatcttgcgcaatggggggaaccctgacgcagcgacgccgcgtgcgggacggaggccttcgggtcgtaaaccgctttcagcagggaagagtcaagactgtacctgcagaagaagccccggctaactacgtgccagcagccgcggtaat

SEQ ID NO: 12 - DNA, 176 bp, synthetic construct
  source 1..176: mol_type = other DNA
actcctacgggaggcagcagggggaatattgcacaatgggcgcaagcctgatgcagcgacgccgcgtgagggatggaggccttcgggttgtaaacctcttttatcggggaacaagcgagagtgagtttacccgttgaataagcaccggctaactacgtgccagcagccgcggtaat

SEQ ID NO: 13 - DNA, 178 bp, synthetic construct
  source 1..178: mol_type = other DNA
actcctacgggaggcagcagggggaatattgcacaatgggcgcaagcctgatgcagcgacgccgcgtgagggatggaggccttcgggttgtaaacctcttttatcggggagcaagccttcgggtgagtgtacctttcgaataagcgccggctaactacgtgccagcagccgcggtaat

SEQ ID NO: 14 - DNA, 180 bp, synthetic construct
  source 1..180: mol_type = other DNA
actcctacgggaggcagcagggggaatattgcacaatgggcgcaagcctgatgcagcgacgccgcgtgcgggatggaggccttcgggttgtaaaccgcttttgatcgggagcaagcccttcggggtgagtgtacccttcgaataagcaccggctaactacgtgccagcagccgcggtaat

SEQ ID NO: 15 - DNA, 178 bp, synthetic construct
  source 1..178: mol_type = other DNA
actcctacgggaggcagcagggggaatcttgcgcaatgggcggaagcctgacgcagcgacgccgcgtgcgggaggaaggccctcgggtcgtaaaccgctttcagcagggacgaggccgcaaggtgacggtacctgcagaagaagccccggctaactacgtgccagcagccgcggtaat

SEQ ID NO: 16 - DNA, 172 bp, synthetic construct
  source 1..172: mol_type = other DNA
actcctacgggaggcagcagggggaattttgcgcaatgggggaaaccctgacgcagcaacgccgcgtgcgggacgacggccttcgggttgtaaaccgctttcagcagggaagaaattcgacggtacctgcagaagaagctccggctaactacgtgccagcagccgcggtaat

SEQ ID NO: 17 - DNA, 181 bp, synthetic construct
  source 1..181: mol_type = other DNA
actcctacgggaggcagcagggggaatattgcacaatgggcgcaagcctgatgcagcgacgccgcgtgcgggatgacggccttcgggttgtaaacctcttttgttagggagcaaggcactttgtgttgagtgtacctttcgaataagcaccggctaactacgtgccagcagccgcggtaat

SEQ ID NO: 18 - DNA, 172 bp, synthetic construct
  source 1..172: mol_type = other DNA
actcctacgggaggcagcagggggaatcttgcgcaatgggggcaaccctgacgcagcgacgccgcgtgcgggacgaaggccttcgggtcgtaaaccgctttcagcagggacgaggcaagacggtacctgcagaagaagccccggctaactacgtgccagcagccgcggtaat

SEQ ID NO: 19 - DNA, 171 bp, synthetic construct
  source 1..171: mol_type = other DNA
actcctacgggaggcagcagggggaatcttgcgcaatggggggaaccctgacgcagcgacgccgcgtgcgggatggaggccttcgggtcgtgaaccgctttcagcagggacgagtctgacggtacctgcagaagaagccccggctaactacgtgccagcagccgcggtaat

SEQ ID NO: 20 - DNA, 178 bp, synthetic construct
  source 1..178: mol_type = other DNA
actcctacgggaggcagcagggggaatcttgcgcaatgggcgaaagcctgacgcagcgacgccgcgtgcgggacgaaggccttctggtcgtaaaccgctttcagcagggacgagggggagacctgacggtacctgcagaagaagccccggctaactacgtgccagcagccgcggtaat

SEQ ID NO: 21 - DNA, 178 bp, synthetic construct
  source 1..178: mol_type = other DNA
actcctacgggaggcagcagggggaatcttgcgaaatgggggcaaccctgacgcagcgacgccgcgtacgggacgaaggccttcgggtcgtaaaccgctttcagcagggacgaggccggaaggtgacggtacctgcagaagaagccccggctaactacgtgccagcagccgcggtaat

SEQ ID NO: 22 - DNA, 178 bp, synthetic construct
  source 1..178: mol_type = other DNA
actcctacgggaggcagcagggggaatattgcacaatgggcgcaagcctgatgcagcgacgccgcgtgcgggatgacggccttcgggttgtaaaccgctttcgatcgggagcaagccttcgggtgagtgtacctttcgaataagcaccggctaactacgtgccagcagccgcggtaat

SEQ ID NO: 23 - DNA, 172 bp, synthetic construct
  source 1..172: mol_type = other DNA
actcctacgggaggcagcagggggaattttgcgcaatgggggcaaccctgacgcagcaacgccgcgtgcgggatgacggccttcgggttgtaaaccgctttcagcagggaagaccgacgacggtacctgcagaagaagccccggctaactacgtgccagcagccgcggtaat

SEQ ID NO: 24 - DNA, 178 bp, synthetic construct
  source 1..178: mol_type = other DNA
actcctacgggaggcagcagggggaaggttgcacaatgggcgaaagcctgatgcagcgacgccgcgtgcgggaaggaggccctcgggtcgtaaaccgctttcagcagggacgaggccgcgaggtgacggtacctgcagaagaagccccggctaactacgtgccagcagccgcggtaat

SEQ ID NO: 25 - DNA, 172 bp, synthetic construct
  source 1..172: mol_type = other DNA
actcctacgggaggcagcagggggaatcttgcgcaatgggggcaaccctgacgcagcgacgccacgtgcgggatggaggccttcgggtcgtaaaccgctttcagcagggacgagtcaagactgtacctgcagaagaagccccggctaactacgtgccagcagccgcggtaat

SEQ ID NO: 26 - DNA, 173 bp, synthetic construct
  source 1..173: mol_type = other DNA
actcctacgggaggcagcagggggaattttgcgcaatgggggcaaccctgacgcagcaacgccgcgtgcgggacgaaggcgtccgcgtcgtaaaccgctttcagcggggaacacctaacgagggtacccgcagaagaagccccggctaaatacgtgccagcagccgcggtaat

SEQ ID NO: 27 - DNA, 171 bp, synthetic construct
  source 1..171: mol_type = other DNA
actcctacgggaggcagcagggggaatattgcgcaatggggggaaccctgacgcagcgacgccgcgtgcgggacggaggccttcgggtcgtaaaccgctttcagcagggaagacatagacggtacctgcagaagaagctccggctaactacgtgccagcagccgcggtaat

SEQ ID NO: 28 - DNA, 172 bp, synthetic construct
  source 1..172: mol_type = other DNA
actcctacgggaggcagcagggggaatcttgcgcaatgggggcaaccctgacgcagcgacgccgcgtgcgggatggaggccttcgggtcgtaaaccgctttcagcagggacgagccaagacggtacctgcagaagaagccccggctaactacgtgccagcagccgcggtaat

SEQ ID NO: 29 - DNA, 188 bp, synthetic construct
  source 1..188: mol_type = other DNA
actcctacgggaggcagcagggggaatattgcacaatgggcgcaagcctgatgcagcgacgccgcgtgcgggatggaggccttcgggttgtaaaccgcttttgttcaagggcaaggcacggtttcggccgtgttgagtggattgttcgaataagcaccggctaactacgtgccagcagccgcggtaat

SEQ ID NO: 30 - DNA, 172 bp, synthetic construct
  source 1..172: mol_type = other DNA
actcctacgggaggcagcagggggaatattgcacaatgggggcaaccctgacgcagcgacgccgcgtgcgggacggaggccttcgggtcgtaaaccgctttcagcagggaagagacaagactgtacctgcagaagaagccccggctaactacgtgccagcagccgcggtaat

SEQ ID NO: 31 - DNA, 171 bp, synthetic construct
  source 1..171: mol_type = other DNA
actcctacgggaggcagcagggggaatattgcacaatgggggaaaccctgatgcagcgacgccgcgtgaaggaagaagtatctcggtatgtaaacttctatcagcagggaagacagtgacggtacctgactaagaagccccggctaactacgtgccagcagccgcggtaat

SEQ ID NO: 32 - DNA, 197 bp, synthetic construct
  source 1..197: mol_type = other DNA
actcctacgggaggcagcagggggaatcttccgcaatggacgaaagtctgacggagcaacgccgcgtgaacgatgacggccttcgggttgtaaagttctgttatacgggacgaatggtacgacggtcaatacccgtcgtaagtgacggtaccgtaagagaaagccacggctaactacgtgccagcagccgcggtaat

SEQ ID NO: 33 - DNA, 197 bp, synthetic construct
  source 1..197: mol_type = other DNA
actcctacgggaggcagcagagggaatcttcggcaatgggggcaaccctgaccgagcaacgccgcgtgagtgaagaaggttttcggatcgtaaagctctgttgtaagtcaagaacgagtgtgagagtggaaagttcacactgtgacggtagcttaccagaaagcgacggctaactacgtgccagcagccgcggtaat

SEQ ID NO: 34 - DNA, 171 bp, synthetic construct
  source 1..171: mol_type = other DNA
actcctacgggaggcagcagggggaatattgcacaatgggggaaaccctgatgcagcgacgccgcgtgagcgatgaagtatttcggtatgtaaagctctatcagcagggaagatagtgacagtacctgactaagaagccccggctaactacgtgccagcagccgcggtaat

SEQ ID NO: 35 - DNA, 197 bp, synthetic construct
  source 1..197: mol_type = other DNA
actcctacgggaggcagcagggggaatcttccgcaatgggcgaaagcctgacggagcaacgccgcgtgaacgatgaaggtcttaggatcgtaaagttctgttgttagggacgaagggtaaggataataataaggtttttatttgacggtacctaacgaggaagccacggctaactacgtgccagcagccgcggtaat

SEQ ID NO: 36 - DNA, 171 bp, synthetic construct
  source 1..171: mol_type = other DNA
actcctacgggaggcagcagggggaatattgcacaatgggggaaaccctgatgcagcgacgccgcgtggaggaagaaggtcttcggattgtaaactcctgttgttgaggaagataatgacggtacccaacaaggaagtgacggctaactacgtgccagcagccgcggtaaa

SEQ ID NO: 37 - DNA, 197 bp, synthetic construct
  source 1..197: mol_type = other DNA
actcctacgggaggcagcagggggaatcttccgcaatggacgaaagtctgacggagcaacgccgcgtgagtgatgaaggatttcggtctgtaaagctctgttgtttatgacgaacgtgcagtgtgtgaacaatgcaatgcaatgacggtagtaaacgaggaagccacggctaactacgtgccagcagccgcggtaat

SEQ ID NO: 38 - DNA, 171 bp, synthetic construct
  source 1..171: mol_type = other DNA
actcctacgggaggcagcagggggaatattgcacaatgggggaaaccctgatgcagcgacgccgcgtgagcgaagaagtatttcggtatgtaaagctctatcagcagggaagataatgacggtacctgactaagaagcaccggctaaatacgtgccagcagccgcggtaat

SEQ ID NO: 39 - DNA, 197 bp, synthetic construct
  source 1..197: mol_type = other DNA
actcctacgggaggcagcagggggaatcttccgcaatggacgaaagtctgacggagcaacgccgcgtgaacgatgacggccttcgggttgtaaagttctgttatatgggacgaacaggataacggttaatacccgatatccctgacggtaccgtaagagaaagccacggctaactacgtgccagcagccgcggtaat

SEQ ID NO: 40 - DNA, 197 bp, synthetic construct
  source 1..197: mol_type = other DNA
actcctacgggaggcagcagggggaatcttccgcaatggacgaaagtctgacggagcaacgccgcgtgagtgatgaaggtcttcggattgtaaaactctgttgttagggacgaaagcaccgtgttcgaacaggtcatggtgttgacggtacctaacgaggaagccacggctaactacgtgccagcagccgcggtaaa

SEQ ID NO: 41 - DNA, 172 bp, synthetic construct
  source 1..172: mol_type = other DNA
actcctacgggaggcagcagggggaatattgcacaatgggggaaaccctgatgcagcgacgccgcgtgagcgaagaagtatttcggtatgtaaagctctatcagcagggaagaaaatgacggtacctgactaagaagccccggctaactacgtgccagcagccgcgtgtaat

SEQ ID NO: 42 - DNA, 171 bp, synthetic construct
  source 1..171: mol_type = other DNA
actcctacgggaggcagcagggggaatattgcacaatgggggaaaccctgatgcagcgacgccgcgtgagcgatgaagtacttcggtatgtaaagctctatcagcagggaagaaaatgacggtacctgactaagaagcaccggctaaatacgtgccagcagccgcggtaat

SEQ ID NO: 43 - DNA, 173 bp, synthetic construct
  source 1..173: mol_type = other DNA
actcctacgggaggcagcagggggaatattgcacaatgggggaaaccctgatgcagcgacgccgcgtgaaggaagaagtatctcggtatgtaaacttctatcagcagggaagaagaaatgacggtacctgactaagaagcaccggctaactacgtgccagcagccgcggtaat

SEQ ID NO: 44 - DNA, 171 bp, synthetic construct
  source 1..171: mol_type = other DNA
actcctacgggaggcagcaggggggacattgcacaatgggggaaaccctgatgcagcgacgccgcgtggaggaagaaggttttcggattgtaaactcctgtcgttagggacgataatgacggtacctaacaagaaagcaccggctaactacgtgccagcagccgcggtaaa

SEQ ID NO: 45 - DNA, 171 bp, synthetic construct
  source 1..171: mol_type = other DNA
actcctacgggaggcagcagggggaatattgcacaatgggggaaaccctgatgcagcgacgccgcgtgagtgaagaagtatctcggtatgtaaagctctatcagcagggaagaaaatgacggtacctgactaagaagccccggctaactacgtgccagcagccgcggtaat

SEQ ID NO: 46 - DNA, 197 bp, synthetic construct
  source 1..197: mol_type = other DNA
actcctacgggaggcagcagggggaatcttccgcaatggacgaaagtctgacggagcaacgccgcgtgagtgaagaaggtcttcggactgtaaaactctgttgttagggacgaaagcagttatgaataacaagtgtagctgttgacggtacctgacgaggaagccacggctaactacgtgccagcagccgcggtaat

SEQ ID NO: 47 - DNA, 197 bp, synthetic construct
  source 1..197: mol_type = other DNA
actcctacgggaggcagcagggggaatcttccgcaatggacgaaagtctgacggagcaacgccgcgtgagtgatgacggccttcgggttgtaaagctctgttaatcgggacgaaaggtcctcttgcgaatagttagaggaattgacggtaccggaatagaaagccacggctaactacgtgccagcagccgcggtaat

SEQ ID NO: 48 - DNA, 173 bp, synthetic construct
  source 1..173: mol_type = other DNA
actcctacgggaggcagcagggggaatattgcacaatgggggaaaccctgatgcagcgacgccgcgtgagcgaagaagtatttcggtatgtaaagctctatcagcagggaagaagaaatgacggtacctgactaagaagcaccggctaaatacgtgccagcagccgcggtaat

SEQ ID NO: 49 - DNA, 172 bp, synthetic construct
  source 1..172: mol_type = other DNA
actcctacgggaggcagcagggggaatattgcacaatgggggaaaccctgatgcagcgacgccgcgtgaaggaagaagtatctcggtatgtaaacttctatcagcagggaagatagtgacggtacctgactaagaagccccggctaacttacgtgccagcagccgcggtaat

SEQ ID NO: 50 - DNA, 172 bp, synthetic construct
  source 1..172: mol_type = other DNA
actcctacgggaggcagcagggggaatcttgcgcaatgggggcaaccctgacgcagcaacgccgcgtgaaggatgaaggttttcggattgtaaacttcttttattaaggacgaaaattgacggtacttaatgaataagctccggctaactacgtgccagcagccgcggtaat

SEQ ID NO: 51 - DNA, 171 bp, synthetic construct
  source 1..171: mol_type = other DNA
actcctacgggaggcagcagggggaatattgcacaatggaggaaactctgatgcagcgacgccgcgtgaaggatgaagtatttcggtatgtaaacttctatcagcagggaagaaaatgacggtacctgactaagaagcaccggctaaatacgtgccagcagccgcggtaat

SEQ ID NO: 52 - DNA, 171 bp, synthetic construct
  source 1..171: mol_type = other DNA
actcctacgggaggcagcagggggaatattgcacaatgggggaaaccctgatgcagcgacgccgcgtgaaggaagaagtatctcggtttgtaaacttctatcagcagggaagataatgacggtacctgactaagaagccccggctaactacgtgccagcagccgcggtaat

SEQ ID NO: 53 - DNA, 171 bp, synthetic construct
  source 1..171: mol_type = other DNA
actcctacgggaggcagcagggggaatcttgcgcaatgggggaaaccctgatgcagcgacgccgcgtgaaggaagaagtatctcggtatgtaaacttctatcagcagggaagatagtgacggtacctgactaagaagccccggctaactacgtgccagcagccgcggtaat

SEQ ID NO: 54 - DNA, 197 bp, synthetic construct
  source 1..197: mol_type = other DNA
actcctacgggaggcagcagagggaatcttcggcaatgggggcaaccctgaccgagcaacgccgcgtgagtgaagaaggttttcggatcgtaaagctctgttgtaagagaagaacgagtgtgagagtggaaagttcacactgtgacggtaacttaccagaaagggacggctaactacgtgccagcagccgcggtaat

SEQ ID NO: 55 - DNA, 196 bp, synthetic construct
  source 1..196: mol_type = other DNA
actcctacgggaggcagcagggggaatattgcacaatgggcgcaagcctgatgcagccatgccgcgtgtatgaagaaggccttcgggttgtaaagtactttcagcggggaggaagggagtaaagttaatacctttactcattgacgttacccgcagaagaagccccggctaactacgtgccagcagccgcggtaat

SEQ ID NO: 56 - DNA, 196 bp, synthetic construct
  source 1..196: mol_type = other DNA
actcctacgggaggcagcagggggaattttggacaatgggcgaaagcctgatccagcaatgccgcgtgtgtgaagaaggccttcgggttgtaaagcacttttgtccggaaagaaatccttggctctaatacagtcgggggatgacgttaccggaagaataagcaccggctaactacgtgccagcagccgcggtaat

SEQ ID NO: 57 - DNA, 195 bp, synthetic construct
  source 1..195: mol_type = other DNA
actcctacgggaggcagcagggggaatattgcacaatgggcgcaagcctgatgcagccatgccgcgtgtgtgaagaaggccttcgggttgtaaagcactttcagcggggaggaaggcggtgaggttaataaccttggcgattgacgttacccgcagaagaagcaccggctaactccgtgcagcagccggggtaat

SEQ ID NO: 58 - DNA, 171 bp, synthetic construct
  source 1..171: mol_type = other DNA
actcctacgggaggcagcagggggaatattggacaatgggggcaaccctgatccagccatgccgcgtgagtgatgaaggccctagggttgtaaagctcttttgtgcgggaagataatgacggtaccgcaagaataagccccggctaacttcgtgccagcagccgcggtaat

SEQ ID NO: 59 - DNA, 196 bp, synthetic construct
  source 1..196: mol_type = other DNA
actcctacgggaggcagcagggggaatattggacaatgggcgcaagcctgatccagccatgccgcgtgggtgaagaaggccttcgggttgtaaagcccttttgttcgggaagaaatcgtctgggctaataccccgggcggatgacggtaccggaagaataagcaccggctaacttcgtgccagcagccgcggtaat

SEQ ID NO: 60 - DNA, 196 bp, synthetic construct
  source 1..196: mol_type = other DNA
actcctacgggaggcagcaggaggaatattgcacaatgggcgcaagcctgatccagctattccgcgtgtgggatgaaggccctcgggttgtaaaccacttttgtagagaacgaaaagacaccttcgaataaagggtgttgctgacggtactctaagaataagcaccggctaactacgtgccagcagccgcggtaat

SEQ ID NO: 61 - DNA, 197 bp, synthetic construct
  source 1..197: mol_type = other DNA
actcctacgggaggcagcagggggaatattgcacaatgggggaaaccctgacgcagcgacgccgcgtgagggatgaaggttctcggatcgtaaacctctgtcaggggggaagaaaccccctcgtgtgaataatgcgagggcttgacggtacccccaaaggaagcaccggctaactccgtgccagcagccgcggtaat

SEQ ID NO: 62 - DNA, 196 bp, synthetic construct
  source 1..196: mol_type = other DNA
actcctacgggaggcagcagggggaattttggacaatgggggcaaccctgatccagccatgccgcgtgcaggatgaaggtcttcggattgtaaactgcttttgtcagggacgaaaagggatgcgataacaccgtattccgctgacggtacctgaagaataagcaccggctaactacgtgccagcagccgcggtaat

SEQ ID NO: 63 - DNA, 196 bp, synthetic construct
  source 1..196: mol_type = other DNA
actcctacgggaggcagcagggggaatattgcgcaatgggggcaaccctgacgcagccatgccgcgtgaatgaagaaggccttcgggttgtaaagttctttcggtagcgaggaaggcatttagtttaatagactaggtgattgacgttaactacagaagaagcaccggctaactccgtgccagcagccgcggtaat

SEQ ID NO: 64 - DNA, 171 bp, synthetic construct
  source 1..171: mol_type = other DNA
actcctacgggaggcagcagggggaatattggacaatgggggcaaccctgatccagccatgccgcgtgagtgatgaaggccttaggtttgtaaagctcttttgtccgggacgataatgacggtaccggaagaataagccccggctaacttcgtgccagcagccgcggtaat

SEQ ID NO: 65 - DNA, 196 bp, synthetic construct
  source 1..196: mol_type = other DNA
actcctacgggaggcagcagggggaatattgcacaatgggcgcaagcctgatgcagccatgccgcgtgtatgaagaaggccttagggttgtaaagtactttcagcggggaggaaggtgataaggttaatacccttgtcaattgacgttacccgcagaagaagcaccggctaactccgtgccagcagccgcggtaat

SEQ ID NO: 66 - DNA, 23 bp, synthetic construct
  source 1..23: mol_type = other DNA
ctggcaccaagcagaagacggca

SEQ ID NO: 67 - DNA, 22 bp, synthetic construct
  source 1..22: mol_type = other DNA
acggcgaccaccgagatctacc

SEQ ID NO: 68 - DNA, 16 bp, synthetic construct
  source 1..16: mol_type = other DNA
ggcaccaagcagaaga

SEQ ID NO: 69 - DNA, 90 bp, synthetic construct
  source 1..90: mol_type = other DNA
tgctggcaccaagcagaagacggcatagctttccaccacacgacatgcatcgcgtagtcctattcggtagatctcggtggtcgccgtatc

SEQ ID NO: 70 - DNA, 90 bp, synthetic construct
  source 1..90: mol_type = other DNA
tgctggcaccaagcagaagacggcatagctttccaccacacaccatgcagcatgtggtcctatccggtagatctcggtggtcgccgtatc

SEQ ID NO: 71 - DNA, 90 bp, synthetic construct
  source 1..90: mol_type = other DNA
tgctggcaccaagcagaagacggcatagctttccaccacacaccatgaagcgcgcggtcctatccggtagatctcggtggtcgccgtatc

SEQ ID NO: 72 - DNA, 90 bp, synthetic construct
  source 1..90: mol_type = other DNA
tgctggcaccaagcagaagacggcatagctttccaccacacaccatgaagcgcgtggtcctatccggtagatctcggtggtcgccgtatc

SEQ ID NO: 73 - DNA, 90 bp, synthetic construct
  source 1..90: mol_type = other DNA
tgctggcaccaagcagaagacggcatagctttccaccacacaccatgcgacatgtggtcctatccggtagatctcggtggtcgccgtatc

SEQ ID NO: 74 - DNA, 90 bp, synthetic construct
  source 1..90: mol_type = other DNA
tgctggcaccaagcagaagacggcatagctttccaccacagcacatgaatgccgtggtcctattcggtagatctcggtggtcgccgtatc

SEQ ID NO: 75 - DNA, 90 bp, synthetic construct
  source 1..90: mol_type = other DNA
tgctggcaccaagcagaagacggcatagctttccaccacacaccattcgatgcgcggtcctatccggtagatctcggtggtcgccgtatc

SEQ ID NO: 76 - DNA, 90 bp, synthetic construct
  source 1..90: mol_type = other DNA
tgctggcaccaagcagaagacggcatagctttccaccacaggacatgcatcccgtggtcctatccggtagatctcggtggtcgccgtatc

SEQ ID NO: 77 - DNA, 90 bp, synthetic construct
  source 1..90: mol_type = other DNA
tgctggcaccaagcagaagacggcatagctttccaccagaagacatgcgtcttgaggtcctatccggtagatctcggtggtcgccgtatc

SEQ ID NO: 78 - DNA, 90 bp, synthetic construct
  source 1..90: mol_type = other DNA
tgctggcaccaagcagaagacggcatagctttccaccacaagacatgcatcccatggtcctatccggtagatctcggtggtcgccgtatc

SEQ ID NO: 79 - DNA, 90 bp, synthetic construct
  source 1..90: mol_type = other DNA
tgctggcaccaagcagaagacggcatagctttccaccacagcacatgcatgccgtggtcctatccggtagatctcggtggtcgccgtatc

SEQ ID NO: 80 - DNA, 90 bp, synthetic construct
  source 1..90: mol_type = other DNA
tgctggcaccaagcagaagacggcatagctttccacctaaagacatgcgcctaaaggtcctatccggtagatctcggtggtcgccgtatc

SEQ ID NO: 81 - DNA, 90 bp, synthetic construct
  source 1..90: mol_type = other DNA
tgctggcaccaagcagaagacggcatagctttccaccacaaggcatgcgccaagtggtcctatccggtagatctcggtggtcgccgtatc

SEQ ID NO: 82 - DNA, 90 bp, synthetic construct
  source 1..90: mol_type = other DNA
tgctggcaccaagcagaagacggcatagctttccaccccaaggcatgcgcctcggggtcctatccggtagatctcggtggtcgccgtatc

SEQ ID NO: 83 - DNA, 90 bp, synthetic construct
  source 1..90: mol_type = other DNA
tgctggcaccaagcagaagacggcatagctttccaccacagaacatgcatcccatggtcctatccggtagatctcggtggtcgccgtatc

SEQ ID NO: 84 - DNA, 90 bp, synthetic construct
  source 1..90: mol_type = other DNA
tgctggcaccaagcagaagacggcatagctttccaccacaagacatgcatcccgtggtcctatccggtagatctcggtggtcgccgtatc

SEQ ID NO: 85 - DNA, 20 bp, synthetic construct
  source 1..20: mol_type = other DNA
agagtttgatymtggctcag

SEQ ID NO: 86 - DNA, 20 bp, synthetic construct
  source 1..20: mol_type = other DNA
agagtttgatymtggctcag

SEQ ID NO: 87 - DNA, 17 bp, synthetic construct
  source 1..17: mol_type = other DNA
cctacgggnggcwgcag

SEQ ID NO: 88 - DNA, 19 bp, synthetic construct
  source 1..19: mol_type = other DNA
gtgccagcmgccgcggtaa

SEQ ID NO: 89 - DNA, 19 bp, synthetic construct
  source 1..19: mol_type = other DNA
gtgccagcmgccgcggtaa

SEQ ID NO: 90 - DNA, 23 bp, synthetic construct
  source 1..23: mol_type = other DNA
gaattgacgggggcccgcacaag

SEQ ID NO: 91 - DNA, 16 bp, synthetic construct
  source 1..16: mol_type = other DNA
caacgagcgcaaccct

SEQ ID NO: 92 - DNA, 18 bp, synthetic construct
  source 1..18: mol_type = other DNA
gctgcctcccgtaggagt

SEQ ID NO: 93 - DNA, 17 bp, synthetic construct
  source 1..17: mol_type = other DNA
attaccgcggctgctgg

SEQ ID NO: 94 - DNA, 21 bp, synthetic construct
  source 1..21: mol_type = other DNA
gactachvgggtatctaatcc

SEQ ID NO: 95 - DNA, 20 bp, synthetic construct
  source 1..20: mol_type = other DNA
ggactachvgggtwtctaat

SEQ ID NO: 96 - DNA, 18 bp, synthetic construct
  source 1..18: mol_type = other DNA
gaattaaaccacatgctc

SEQ ID NO: 97 - DNA, 24 bp, synthetic construct
  source 1..24: mol_type = other DNA
cggtgtgtacaaggcccgggaacg

SEQ ID NO: 98 - DNA, 22 bp, synthetic construct
  source 1..22: mol_type = other DNA
tacggytaccttgttacgactt

SEQ ID NO: 99 - DNA, 19 bp, synthetic construct
  source 1..19: mol_type = other DNA
gtagtcatatgcttgtctc

SEQ ID NO: 100 - DNA, 21 bp, synthetic construct
  source 1..21: mol_type = other DNA
gagacaagcatatgactactg

SEQ ID NO: 101 - DNA, 21 bp, synthetic construct
  source 1..21: mol_type = other DNA
ggctgctggcaccagacttgc

SEQ ID NO: 102 - DNA, 21 bp, synthetic construct
  source 1..21: mol_type = other DNA
gcaagtctggtgccagcagcc

SEQ ID NO: 103 - DNA, 20 bp, synthetic construct
  source 1..20: mol_type = other DNA
cttccgtcaattcctttaag

SEQ ID NO: 104 - DNA, 22 bp, synthetic construct
  source 1..22: mol_type = other DNA
aacttaaaggaattgacggaag

SEQ ID NO: 105 - DNA, 24 bp, synthetic construct
  source 1..24: mol_type = other DNA
gcatcacagacctgttattgcctc

SEQ ID NO: 106 - DNA, 24 bp, synthetic construct
  source 1..24: mol_type = other DNA
gaggcaataacaggtctgtgatgc

SEQ ID NO: 107 - DNA, 20 bp, synthetic construct
  source 1..20: mol_type = other DNA
tccgcaggttcacctacgga

SEQ ID NO: 108 - DNA, 16 bp, synthetic construct
  source 1..16: mol_type = other DNA
taagccatgcatgtct

SEQ ID NO: 109 - DNA, 17 bp, synthetic construct
  source 1..17: mol_type = other DNA
gcggtaattccagctcc

SEQ ID NO: 110 - DNA, 18 bp, synthetic construct
  source 1..18: mol_type = other DNA
ggagtggagcctgcggct

SEQ ID NO: 111 - DNA, 17 bp, synthetic construct
  source 1..17: mol_type = other DNA
aagtcgtaacaaggttt

SEQ ID NO: 112 - DNA, 17 bp, synthetic construct
  source 1..17: mol_type = other DNA
aaaccttgttacgactt

SEQ ID NO: 113 - DNA, 19 bp, synthetic construct
  source 1..19: mol_type = other DNA
catgtctaagtttaagcaa

SEQ ID NO: 114 - DNA, 20 bp, synthetic construct
  source 1..20: mol_type = other DNA
ctcattccaattacaagacc

SEQ ID NO: 115 - DNA, 22 bp, synthetic construct
  source 1..22: mol_type = other DNA
ccggagaaggagcctgagaaac

SEQ ID NO: 116 - DNA, 20 bp, synthetic construct
  source 1..20: mol_type = other DNA
cgtccctattaatcattacg

SEQ ID NO: 117 - DNA, 18 bp, synthetic construct
  source 1..18: mol_type = other DNA
gaataatagaataggacg

SEQ ID NO: 118 - DNA, 21 bp, synthetic construct
  source 1..21: mol_type = other DNA
aatatacgctattggagctgg

SEQ ID NO: 119 - DNA, 20 bp, synthetic construct
  source 1..20: mol_type = other DNA
aattaagcagacaaatcact

SEQ ID NO: 120 - DNA, 19 bp, synthetic construct
  source 1..19: mol_type = other DNA
gactcaacacgggaaactc

SEQ ID NO: 121 - DNA, 20 bp, synthetic construct
  source 1..20: mol_type = other DNA
aaaccttgttacgactttta

SEQ ID NO: 122 - DNA, 23 bp, synthetic construct
  source 1..23: mol_type = other DNA
gtggtaattctagagctaatact

SEQ ID NO: 123 - DNA, 24 bp, synthetic construct
  source 1..24: mol_type = other DNA
atgtattagctctagaattaccac

SEQ ID NO: 124 - DNA, 19 bp, synthetic construct
  source 1..19: mol_type = other DNA
ctgccctatcaactttcga

SEQ ID NO: 125 - DNA, 19 bp, synthetic construct
  source 1..19: mol_type = other DNA
tcgaaagttgatagggcag

SEQ ID NO: 126 - DNA, 23 bp, synthetic construct
  source 1..23: mol_type = other DNA
gtctagtataatcgttatacagg

SEQ ID NO: 127 - DNA, 20 bp, synthetic construct
  source 1..20: mol_type = other DNA
ggagtatggtcgcaaggctg

SEQ ID NO: 128 - DNA, 20 bp, synthetic construct
  source 1..20: mol_type = other DNA
cagccttgcgaccatactcc

SEQ ID NO: 129 - DNA, 20 bp, synthetic construct
  source 1..20: mol_type = other DNA
gtgagtttccccgtgttgag

SEQ ID NO: 130 - DNA, 20 bp, synthetic construct
  source 1..20: mol_type = other DNA
ttgctacatggataactgtg

SEQ ID NO: 131 - DNA, 18 bp, synthetic construct
  source 1..18: mol_type = other DNA
ctgttaagactacaacgg

SEQ ID NO: 132 - DNA, 18 bp, synthetic construct
  source 1..18: mol_type = other DNA
agagtgttcaaagcaggc

SEQ ID NO: 133 - DNA, 21 bp, synthetic construct
  source 1..21: mol_type = other DNA
ctcactaagccattcaatcgg

SEQ ID NO: 134 - DNA, 20 bp, synthetic construct
  source 1..20: mol_type = other DNA
  misc_difference 19: replace = t; replace = c
tctagagctaatacatgcng

SEQ ID NO: 135 - DNA, 20 bp, synthetic construct
  source 1..20: mol_type = other DNA
ggccctcaaatctaaggatt

SEQ ID NO: 136 - DNA, 20 bp, synthetic construct
  source 1..20: mol_type = other DNA
aatttgcgcgcctgctgcaa

SEQ ID NO: 137 - DNA, 20 bp, synthetic construct
  source 1..20: mol_type = other DNA
cgtatattaaaattgttgac

SEQ ID NO: 138 - DNA, 20 bp, synthetic construct
  source 1..20: mol_type = other DNA
gactacgagctttttaacgt

SEQ ID NO: 139 - DNA, 20 bp, synthetic construct
  source 1..20: mol_type = other DNA
tttcgcagtagtttgtctta

SEQ ID NO: 140 - DNA, 20 bp, synthetic construct
  source 1..20: mol_type = other DNA
caaactactgcgaaagcatc

SEQ ID NO: 141 - DNA, 20 bp, synthetic construct
  source 1..20: mol_type = other DNA
caaactactgcgaaagcatc

(19) United States
(12) Patent Application Publication
(10) Pub. No.: US 2025/0115967 A1
(43) Pub. Date: Apr. 10, 2025

(54) A Method of Profiling a Microbiotic Composition

(71) Applicant: National University of Singapore, Singapore (SG)

(72) Inventors: Yongqiang Luo, Singapore (SG); Xu Cui, Singapore (SG); Lih Feng Cheow, Singapore (SG)

(21) Appl. No.: 18/836,142

(22) PCT Filed: Feb. 28, 2023
(86) PCT No.: PCT/SG2023/050120

§ 371 Date: Aug. 6, 2024

(30) Foreign Application Priority Data
Feb. 28, 2022 (SG) 10202201933W

(51) Int. Cl. C12Q 1/689; C12Q 1/6895; G01N 21/64

(52) U.S. Cl. CPC C12Q 1/689; C12Q 1/6895; G01N 21/6428; C12Q 2600/156; G01N 2021/6439

(57) ABSTRACT

There is provided a method of profiling a microbiotic composition of a sample comprising the nucleic acid molecules of the microbiotic composition, the method comprising a) partitioning the sample in a sufficient number of partitions such that at least a portion of the partitions comprises no more than one nucleic acid molecule of the microbiotic composition; b) contacting the partitions of the sample to a plurality of mismatch-tolerant probes that are capable of binding to the nucleic acid molecules or parts thereof under suitable conditions; c) determining signals generated by each of the plurality of mismatch-tolerant probes in each partition; and d) establishing the microbiotic composition in the sample based on the signals. Also disclosed is a method of determining a health and kits for uses in the methods as described herein.

### TECHNICAL FIELD

The present disclosure relates broadly to the analysis of microbiotic composition of a sample, particularly profiling the nucleic acid molecules to identify microbiotic makeup in the sample.

### BACKGROUND

In recent years, researchers have found that changes in the body's microbial ecosystems are linked to a whole range of different medical conditions, from cancer to gastrointestinal problems and even neurological diseases like Parkinson's. There is great potential for developing microbiome-based diagnostics, as these diseases are now found to be characterized by strongly altered microbiome diversity. A particular advantage of microbiome-based diagnostics is that sample such as stool is easy to collect unlike colonoscopy and biopsy, and hence such procedures are highly amenable to longitudinal sampling.

Nevertheless, microbiome diagnostics presents a unique set of challenges. Whereas conventional disease is typically based on one or a few biomarkers that can be assayed with rapid platforms such as enzyme-linked immunosorbent assay (ELISA) or polymerase chain reaction (PCR), microbiome diagnostics need to rely on the identification of hundreds to thousands of bacteria species that colonize a niche. While rapid molecular fingerprinting approaches such as denaturing gradient gel and amplicon length polymorphism assays exist, they are often neither quantitative nor sensitive enough to detect subtle changes in bacteria diversity. Today, microbiome analysis is largely performed with next generation sequencing (NGS) approaches, in particular 16S ribosomal DNA (16S rDNA) sequencing. Despite being the standard method for microbiome analysis, NGS methods are far from clinical implementation as they require very specialized equipment, involve lengthy and complex workflow, and is only affordable with batch sequencing of large number of samples. There is a clear unmet need for a rapid, multiplexed and cost-effective microbiome profiling platform in clinical laboratories.

Inherent to the challenges of microbiome profiling is the need for very accurate quantification of particular microbiotic species or taxonomy group, and to compare their abundance with various other taxonomy groups present. Digital Polymerase Chain Reaction (dPCR), where highly parallel sample partitioning is coupled with individual PCR reactions in each compartment, is a very promising platform for highly quantitative absolute nucleic acid counting. While nucleic acid counting is highly attractive for individual species abundance measurement, current dPCR technology has limited capability for multidimensional profiling of complex samples such as the microbiome. To date, 5-plex is the maximum multiplexing capability for dPCR, a limitation imposed by the distinct fluorescence channels (2-3) in commercial instruments.

As such, there is a need to provide for an alternative method of profiling a microbiome composition in a sample.

### SUMMARY

In one aspect, there is provided a method of profiling a microbiotic composition of a sample comprising the nucleic acid molecules of the microbiotic composition, the method comprising:
a) partitioning the sample into a sufficient number of partitions such that at least a portion of the partitions comprises no more than one nucleic acid molecule of the microbiotic composition;
b) contacting the partitions of the sample to a plurality of mismatch-tolerant probes that are capable of binding to the nucleic acid molecules or parts thereof under suitable conditions;
c) determining signals generated by each of the plurality of mismatch-tolerant probes in each partition; and
d) establishing the microbiotic composition in the sample based on the signals.
   In some examples, the plurality of mismatch-tolerant probes is configured to collectively generate different signal intensity profiles for different groups of nucleic acid molecules. In some examples, step d) of the method comprises computing a melting temperature (Tm) of each of the plurality of mismatch-tolerant probes to obtain a Tm signature for each partition based on the signals generated by each of the plurality of mismatch-tolerant probes in each partition.

In some examples, computing the Tm comprises identifying an inflection point in a signal intensity generated by each of the plurality of mismatch-tolerant probes in each partition.

In some examples, step d) of the method further comprises classifying the Tm signature as belonging to a group of nucleic acid molecules.

In some examples, step d) of the method further comprises counting the number of partitions with the same Tm signature.

In some examples, step d) of the method further comprises determining a proportion of different groups of nucleic acid molecules in the sample based on the count numbers.

In some examples, the amplification reaction comprises an asymmetric polymerase chain reaction (PCR).

In some examples, the asymmetric PCR uses a primer set comprising:
a pair of forward and reverse primers for amplifying the nucleic acid molecules or parts thereof to produce double-stranded PCR products; and
a third primer for amplifying one of the two strands of the double-stranded PCR products or a part thereof.

In some examples, wherein the forward and reverse primers have a higher annealing temperature than the third primer.

In some examples, the third primer is present at a higher concentration than the forward and reverse primers.

In some examples, the plurality of mismatch-tolerant probes is configured to bind to a 16s and/or 18s ribosomal region of nucleic acid region of the microbiotic composition at temperatures below their melting temperature.

In some examples, the plurality of mismatch-tolerant probes is configured to bind to a V3 region of a 16s and/or 18s ribosomal nucleic acid region of the nucleic acid molecules at temperatures below their melting temperatures.

In some examples, the plurality of mismatch-tolerant probes is configured to have different Tm signatures for different groups of nucleic acid molecules, optionally
the plurality of mismatch-tolerant probes is configured to have different Tm signatures for nucleic acid molecules from different phyla and/or species of bacteria and/or fungus.

In some examples, the plurality of mismatch-tolerant probes has one or more of the following properties:
is capable of producing a fluorescent signal;
has a greater number of mismatches with the sequences of one group of nucleic acid molecules than another group of nucleic acid molecules;
comprises an oligonucleotide comprising a reporter moiety and a quencher moiety;
comprises a molecular beacon;
each is capable of producing a distinct colorimetric signal, optionally wherein the colorimetric signal is selected from the group consisting of: a green signal, an orange signal and a red signal.

In some examples, the microbiotic composition comprises one or more microbes comprising bacteria, fungi, and/or combination thereof.

In some examples, the microbiotic composition comprises microbes from one or more bacteria from the genus of Acetobacter, Acinetobacter, Actinomyces, Agrobacterium spp., Azorhizobium, Azotobacter, Anaplasma spp., Bacillus spp., Bacteroides spp., Bartonella spp., Bordetella spp., Borrelia, Brucella spp., Burkholderia spp., Calymmatobacterium, Campylobacter, Chlamydia spp., Chlamydophila spp., Clostridium spp., Corynebacterium spp., Coxiella, Ehrlichia, Enterobacter, Enterococcus spp., Escherichia, Francisella, Fusobacterium, Gardnerella, Haemophilus spp., Helicobacter, Klebsiella, Lactobacillus spp., Lactococcus, Legionella, Listeria, Methanobacterium extroquens, Microbacterium multiforme, Micrococcus luteus, Moraxella catarrhalis, Mycobacterium spp., Mycoplasma spp., Neisseria spp., Pasteurella spp., Peptostreptococcus, Porphyromonas, Pseudomonas, Rhizobium, Rickettsia spp., Rochalimaea spp., Rothia, Salmonella spp., Serratia, Shigella, Staphylococcus spp., Stenotrophomonas, Streptococcus spp., Treponema spp., Vibrio spp., Wolbachia, and Yersinia spp, and/or
one or more fungus from the genus Absidia, Ajellomyces, Arthroderma, Aspergillus, Blastomyces, Candida, Cladophialophora, Coccidioides, Cryptococcus, Cunninghamella, Epidermophyton, Exophiala, Filobasidiella, Fonsecaea, Fusarium, Geotrichum, Histoplasma, Hortaea, Issatschenkia, Madurella, Malassezia, Microsporum, Microsporidia, Mucor, Nectria, Paecilomyces, Paracoccidioides, Penicillium, Pichia, Pneumocystis, Pseudallescheria, Rhizopus, Rhodotorula, Scedosporium, Schizophyllum, Sporothrix, Trichophyton, and Trichosporon.

In some examples, the microbiotic composition comprises microbes from one or more bacteria from the group consisting of Acetobacter aurantius, Acinetobacter baumannii, Actinomyces Israelii, Agrobacterium radiobacter, Agrobacterium tumefaciens, Azorhizobium caulinodans, Azotobacter vinelandii, Anaplasma phagocytophilum, Anaplasma marginale, Bacillus anthracis, Bacillus brevis, Bacillus cereus, Bacillus fusiformis, Bacillus licheniformis, Bacillus megaterium, Bacillus mycoides, Bacillus stearothermophilus, Bacillus subtilis, Bacteroides fragilis, Bacteroides gingivalis, Bacteroides melaminogenicus (Prevotella melaminogenica), Bartonella henselae, Bartonella quintana, Bordetella bronchiseptica, Bordetella pertussis, Borrelia burgdorferi, Brucella abortus, Brucella melitensis, Brucella suis, Burkholderia mallei, Burkholderia pseudomallei, Burkholderia cepacia complex, Burkholderia cenocepacia, Calymmatobacterium granulomatis, Campylobacter coli, Campylobacter fetus, Campylobacter jejuni, Campylobacter pylori, Chlamydia trachomatis, Chlamydophila. (such as C. pneumoniae, Chlamydophila psittaci, Clostridium botulinum, Clostridium difficile, Clostridium perfringens, Clostridium tetani), Corynebacterium diphtheriae, Corynebacterium fusiforme, Coxiella bumetii, Ehrlichia chaffeensis, Enterobacter cloacae, Enterococcus avium, Enterococcus durans, Enterococcus faecalis, Enterococcus faecium, Enterococcus galllinarum, Enterobacter gergoviae (now known as Pluralibacter gergoviae), Enterococcus maloratus, Escherichia coli, Francisella tularensis, Fusobacterium nucleatum, Gardnerella vaginalis, Haemophilus ducreyi, Haemophilus influenzae, Haemophilus parainfluenzae, Haemophilus pertussis, Haemophilus vaginalis, Helicobacter pylori, Klebsiella pneumoniae, Lactobacillus acidophilus, Lactobacillus casei, Lactococcus lactis, Legionella pneumophila, Listeria monocytogenes, Methanobacterium extroquens, Microbacterium multiforme, Micrococcus luteus, Moraxella catarrhalis, Mycobacterium avium, Mycobacterium bovis, Mycobacterium diphtheriae, Mycobacterium intracellulare, Mycobacterium leprae, Mycobacterium lepraemurium, Mycobacterium phlei, Mycobacterium smegmatis, Mycobacterium tuberculosis, Mycoplasma fermentans, Mycoplasma genitalium, Mycoplasma hominis, Mycoplasma penetrans, Mycoplasma pneumoniae, Neisseria gonorrhoeae, Neisseria meningitidis, Pasteurella multocida, Pasteurella tularensis Peptostreptococcus, Porphyromonas gingivalis, Pseudomonas aeruginosa, Rhizobium Radiobacter, Rickettsia prowazekii, Rickettsia psittaci, Rickettsia quintana, Rickettsia rickettsii, Rickettsia trachomae, Rochalimaea henselae, Rochalimaea quintana, Rothia dentocariosa, Salmonella enteritidis, Salmonella typhi, Salmonella typhimurium, Serratia marcescens, Shigella dysenteriae, Staphylococcus aureus, Staphylococcus epidermidis, Stenotrophomonas maltophilia, Streptococcus agalactiae, Streptococcus. avium, Streptococcus bovis, Streptococcus cricetus, Streptococcus faceium, Streptococcus faecalis, Streptococcus ferus, Streptococcus gallinarum, Streptococcus lactis, Streptococcus mitior, Streptococcus mitis, Streptococcus mutans, Streptococcus oralis, Streptococcus pneumoniae, Streptococcus pyogenes, Streptococcus rattus, Streptococcus salivarius, Streptococcus sanguis, Streptococcus sobrinus, Treponema pallidum, Treponema denticola, Vibrio cholerae, Vibrio comma, Vibrio parahaemolyticus, Vibrio vulnificus, Wolbachia, Yersinia enterocolitica, Yersinia pestis and Yersinia pseudotuberculosis,
and/or one or more fungus from the group consisting of Absidia corymbifera, Ajellomyces capsulatus, Ajellomyces dermatitidis, Arthroderma benhamiae, Arthroderma fulvum, Arthroderma gypseum, Arthroderma incurvatum, Arthroderma otae and Arthroderma vanbreuseghemii, Aspergillus flavus, Aspergillus fumigatus and Aspergillus niger, Blastomyces dermatitidis, Candida albicans, Candida glabrata, Candida guilliermondii, Candida krusei, Candida parapsilosis, Candida tropicalis and Candida pelliculosa, Cladophialophora carrionii, Coccidioides immitis and Coccidioides posadasii, Cryptococcus neoformans, Cunninghamella Sp, Epidermophyton floccosum, Exophiala dermatitidis, Filobasidiella neoformans, Fonsecaea pedrosoi, Fusarium solani, Geotrichum candidum, Histoplasma capsulatum, Hortaea werneckii, Issatschenkia orientalis, Madurella grisae, Malassezia furfur, Malassezia globosa, Malassezia obtusa, Malassezia pachydermatis, Malassezia restricta, Malassezia slooffiae, Malassezia sympodialis, Microsporum canis, Microsporum fulvum, Microsporum gypseum, Microsporidia, Mucor circinelloides, Nectria haematococca, Paecilomyces variotii, Paracoccidioides brasiliensis, Penicillium marneffei, Pichia anomala, Pichia guilliermondii, Pneumocystis jiroveci, Pneumocystis carinii, Pseudallescheria boydii, Rhizopus oryzae, Rhodotorula rubra, Scedosporium apiospermum, Schizophyllum commune, Sporothnx schenckii, Trichophyton mentagrophytes, Trichophyton rubrum, Trichophyton verrucosum and Trichophyton violaceum, and Trichosporon asahii, Trichosporon cutaneum, Trichosporon inkin and Trichosporon mucoides, and/or
combinations of bacteria and/or fungus thereof.

In some examples, the sample is a medical sample and/or a non-medical sample, optionally
wherein the non-medical sample is a sample of one or more selected from the group consisting of food industries, consumer products, agriculture, and laboratory, or
wherein the medical sample is a biological sample.

In yet another aspect, there is provided a microbiotic composition profiling kit comprising
a reagent for detecting a nucleic acid molecules of a microbiotic composition,
wherein the reagent comprises a plurality of mismatch-tolerant probes configured to have different Tm signatures for different groups of nucleic acid molecules of the microbiotic composition.

### Definitions

The term “nucleic acid” refers to a DNA molecule (for example, but not limited to cDNA or genomic DNA), an RNA molecule (for example, but not limited to an mRNA), or a DNA or RNA analog. A DNA or RNA analog can be synthesized from nucleotide analogs. The nucleic acid molecule can be single-stranded or double-stranded. An “isolated” nucleic acid is a nucleic acid, the structure of which is not identical to that of any naturally occurring nucleic acid or to that of any fragment of a naturally occurring genomic nucleic acid. The term therefore covers, for example, (a) a DNA which has the sequence of part of a naturally occurring genomic DNA molecule but is not flanked by both of the coding sequences that flank that part of the molecule in the genome of the organism in which it naturally occurs; (b) a nucleic acid incorporated into a vector or into the genomic DNA of a prokaryote or eukaryote in a manner such that the resulting molecule is not identical to any naturally occurring vector or genomic DNA; (c) a separate molecule such as a cDNA, a genomic fragment, a fragment produced by PCR, or a restriction fragment; and (d) a recombinant nucleotide sequence that is part of a hybrid gene, i.e., a gene encoding a fusion protein.

“Target nucleic acid” or “target” refers to a nucleic acid containing a target nucleic acid sequence of interest. A target nucleic acid may be single-stranded or double-stranded. A “target nucleic acid sequence,” “target sequence” or “target region” means a specific sequence that comprises all or part of the sequence of a single-stranded nucleic acid. A target sequence may be within a nucleic acid template or within the genome of a cell, which may be any form of single-stranded or double-stranded nucleic acid. A template may be a purified or isolated nucleic acid or may be non-purified or non-isolated.

“Complementary” sequences, may include, or be formed entirely from, Watson-Crick base pairs (e.g., A-T/U and C-G), non-Watson-Crick base pairs and/or base pairs formed from non-natural and modified nucleotides, and in as far as the above requirements with respect to their ability to hybridize are fulfilled. A full complement or fully complementary may mean 100% (completely) complementary or substantially complementary base pairing between nucleotides or nucleotide analogs of nucleic acid molecules.

“Substantially complementary” refers to nucleic acid or oligonucleotide that has a sequence containing at least 10 contiguous bases that are at least 80%, (e.g., 85%, 90%, 95%, 96%, 97%, 98%, 99%, and 100%) to at least 10 contiguous bases in a target nucleic acid sequence so that the nucleic acid or oligonucleotide can hybridize or anneal to the target nucleic acid sequence under, e.g., the annealing condition of a PCR reaction or probe-target hybridization condition. Complementarity between sequences may be expressed a number of base mismatches in each set of at least 10 contiguous bases being compared. The term “substantially identical” means that a first nucleic acid is at least 80%, (e.g., 85%, 90%, 95%, 96%, 97%, 98%, 99%, and 100%) complementary to a second nucleic acid so that the first nucleic acid is substantially complementary to and is capable of hybridizing to the complement of the second nucleic acid under PCR annealing or probe-target hybridization conditions.

“Hybridization” or “hybridizing” or “hybridize” or “anneal” refers to the ability of completely or partially complementary nucleic acid strands to come together under specified hybridization conditions in a parallel or antiparallel orientation to form a stable double-stranded structure or region (sometimes called a “hybrid” or “duplex” or “stem”) in which the two constituent strands are joined by hydrogen bonds. Although hydrogen bonds typically form between adenine and thymine or uracil (A and T or U) or cytosine and guanine (C and G), other base pairs may form.

“Amplification” and its variants, as used herein, includes any process for producing multiple copies or complements of at least some portion of a polynucleotide, the polynucleotide typically being referred to as a “template.” The template polynucleotide can be single stranded or double stranded. A template may be a purified or isolated nucleic acid, or may be non-purified or non-isolated. Amplification of a given template can result in the generation of a population of polynucleotide amplification products, collectively referred to as an “amplicon.” The polynucleotides of the amplicon can be single stranded or double stranded, or a mixture of both. Typically, the template will include a target sequence, and the resulting amplicon will include polynucleotides having a sequence that is either substantially identical or substantially complementary to the target sequence. In some examples, the polynucleotides of a particular amplicon are substantially identical, or substantially complementary, to each other; alternatively, in some examples the polynucleotides within a given amplicon can have nucleotide sequences that vary from each other. Amplification can proceed in linear or exponential fashion, and can involve repeated and consecutive replications of a given template to form two or more amplification products. Some typical amplification reactions involve successive and repeated cycles of template-based nucleic acid synthesis, resulting in the formation of a plurality of daughter polynucleotides containing at least some portion of the nucleotide sequence of the template and sharing at least some degree of nucleotide sequence identity (or complementarity) with the template. In some examples, each instance of nucleic acid synthesis, which can be referred to as a “cycle” of amplification, includes creating free 3′ end (e.g., by nicking one strand of a dsDNA) thereby generating a primer and primer extension steps; optionally, an additional denaturation step can also be included wherein the template is partially or completely denatured. In some examples, one round of amplification includes a given number of repetitions of a single cycle of amplification. For example, a round of amplification can include 5, 10, 15, 20, 25, 30, 35, 40, 50, or more repetitions of a particular cycle. In some examples, amplification includes any reaction wherein a particular polynucleotide template is subjected to two consecutive cycles of nucleic acid synthesis. The synthesis can include template-dependent nucleic acid synthesis.

“Primer” or “primer oligonucleotide” refers to a strand of nucleic acid or an oligonucleotide capable of hybridizing to a template nucleic acid and acting as the initiation point for incorporating extension nucleotides according to the composition of the template nucleic acid for nucleic acid synthesis. “Extension nucleotides” refer to any nucleotides (e.g., dNTP) and analogs thereof capable of being incorporated into an extension product during amplification, i.e., DNA, RNA, or a derivative of DNA or RNA, which may include a label. As used herein, the term “oligonucleotide” refers to a short polynucleotide, typically less than or equal to 300 nucleotides long (e.g., in the range of 5 and 150, preferably in the range of 10 to 100, more preferably in the range of 15 to 50 nucleotides in length). However, as used herein, the term is also intended to encompass longer or shorter polynucleotide chains. An “oligonucleotide” may hybridize to other polynucleotides, therefore serving as a probe for polynucleotide detection, or a primer for polynucleotide chain extension.

“Probe” as used herein refers to an oligonucleotide capable of binding to a target nucleic acid of complementary sequence through one or more types of chemical bonds, usually through complementary base pairing, usually through hydrogen bond formation. Probes may bind target sequences lacking complete complementarity with the probe sequence depending upon the stringency of the hybridization conditions. There may be any number of base pair mismatches which will interfere with hybridization between the target sequence and the single stranded nucleic acids described herein. However, if the number of mutations is so great that no hybridization can occur under even the least stringent of hybridization conditions, the sequence is not a complementary target sequence. A probe may be single stranded or partially single and partially double stranded. The strandedness of the probe is dictated by the structure, composition, and properties of the target sequence. Probes may be directly labelled or indirectly labelled with a label such as with biotin to which a streptavidin complex may later bind.

The term “detection probe” refers to an oligonucleotide having a sequence sufficiently complementary to its target sequence to form a probe:target hybrid stable for detection under stringent hybridization conditions. A probe is typically a synthetic oligomer that may include bases complementary to sequence outside of the targeted region which do not prevent hybridization under stringent hybridization conditions to the target nucleic acid. A sequence non-complementary to the target may be a homopolymer tract (e.g., poly-A or poly-T), promoter sequence, restriction endonuclease recognition sequence, or sequence to confer desired secondary or tertiary structure (e.g., a catalytic site or hairpin structure), or a tag region which may facilitate detection and/or amplification. “Stable” or “stable for detection” means that the temperature of a reaction mixture is at least 2° C. below the melting temperature (Tm) of a nucleic acid duplex contained in the mixture, more preferably at least 5° C. below the Tm, and even more preferably at least 10° C. below the Tm.

A “label” or “reporter molecule” is chemical or biochemical moiety useful for labelling a nucleic acid (including a single nucleotide), polynucleotide, oligonucleotide, or protein ligand, e.g., amino acid or antibody. Examples include fluorescent agents, chemiluminescent agents, chromogenic agents, quenching agents, radionucleotides, enzymes, substrates, cofactors, inhibitors, magnetic particles, and other moieties known in the art. Labels or reporter molecules are capable of generating a measurable signal and may be covalently or noncovalently joined to an oligonucleotide or nucleotide (e.g., a non-natural nucleotide) or ligand.

“Contacting” and its variants, when used in reference to any set of components, includes any process whereby the components to be contacted are mixed into same mixture (for example, are added into the same compartment or solution), and does not necessarily require actual physical contact between the recited components. The recited components can be contacted in any order or any combination (or sub-combination), and can include situations where one or some of the recited components are subsequently removed from the mixture, optionally prior to addition of other recited components. For example, “contacting A with B and C” includes any and all of the following situations: (i) A is mixed with C, then B is added to the mixture; (ii) A and B are mixed into a mixture; B is removed from the mixture, and then C is added to the mixture; and (iii) A is added to a mixture of B and C. “Contacting” a target nucleic acid or a cell with one or more reaction components, such as a polymerase, a primer set or a probe, includes any or all of the following situations: (i) the target or cell is contacted with a first component of a reaction mixture to create a mixture; then other components of the reaction mixture are added in any order or combination to the mixture; and (ii) the reaction mixture is fully formed prior to mixture with the target or cell.

The term “mixture” as used herein, refers to a combination of elements, that are interspersed and not in any particular order. A mixture is heterogeneous and not spatially separable into its different constituents. Examples of mixtures of elements include a number of different elements that are dissolved in the same aqueous solution, or a number of different elements attached to a solid support at random or in no particular order in which the different elements are not spatially distinct. In other words, a mixture is not addressable.

The term “microbiome”, or its permutations such as microbiotic composition, microbiotes, and the like, as used herein refers to a collective of microorganisms, including bacteria, archaea, fungi, viruses, and the like that live in an environment. In the present disclosure, the environment is used broadly to cover any biological environment, such as the gut of a human, and any non-biological environment, such as water bodies or consumer/industrial products.

As used herein, the term “subject” refers to any organism having a genome, preferably, a living animal, e.g., a mammal, which has been the object of diagnosis, treatment, observation or experiment. Examples of a subject can be a human, a livestock animal (beef and dairy cattle, sheep, poultry, swine, etc.), or a companion animal (dogs, cats, horses, etc).

A “sample” as used herein means any medical or non-medical sample. Medical sample may include medical instruments, environment, biological fluid, or tissue obtained from an organism (e.g., patient) or from components (e.g., blood) of an organism. The sample may be of any biological tissue, cell(s) or fluid. The sample may be a “clinical sample” which is a sample derived from a subject, such as a human patient or veterinary subject. Useful biological samples include, without limitation, whole blood, saliva, urine, synovial fluid, bone marrow, cerebrospinal fluid, vaginal mucus, cervical mucus, nasal secretions, sputum, semen, amniotic fluid, bronchoalveolar lavage fluid, faeces, and other cellular exudates from a patient or subject. Such samples may further be diluted with saline, buffer or a physiologically acceptable diluent. Alternatively, such samples are concentrated by conventional means. Biological samples may also include sections of tissues such as frozen sections taken for histological purposes. A biological sample may also be referred to as a “patient sample.” A biological sample may also include a substantially purified or isolated protein, membrane preparation, or cell culture. Examples of non-medical sample include a sample found in food industries (such as in food preparation, preservatives, additives, and the like), consumer products (such as in shampoo, cream, moisturizer, hand sanitizer, soaps, and the like), agriculture (such as in soil composition, water-bodies fertilizer composition, additives, meat or poultry samples, samples from sources of potential contamination or environment of interest (environmental), and the like), or laboratory (such as experimental animals including rodents (rats, mice, and the like), rabbits, non-human primates, and the like).

The terms “determining,” “measuring,” “assessing,” and “assaying” are used interchangeably and include both quantitative and qualitative measurement, and include determining if a characteristic, trait, or feature is present or not. Assessing may be relative or absolute. Assessing the presence of a target includes determining the amount of the target present, as well as determining whether it is present or absent.

As used herein the term “reference” value refers to a value that statistically correlates to a particular outcome when compared to an assay result. In preferred embodiments, the reference value can be determined from statistical analysis that examines the mean of wild type values. The reference value may be a threshold score value or a cutoff score value. Typically a reference value will be a threshold above (or below) which one outcome is more probable and below which an alternative outcome is more probable.

The term “particle” as used herein broadly refers to a discrete entity or a discrete body. The particle described herein can include an organic, an inorganic or a biological particle. The particle used described herein may also be a macro-particle that is formed by an aggregate of a plurality of sub-particles or a fragment of a small object. The particle of the present disclosure may be spherical, substantially spherical, or non-spherical, such as irregularly shaped particles or ellipsoidally shaped particles. The term “size” when used to refer to the particle broadly refers to the largest dimension of the particle. For example, when the particle is substantially spherical, the term “size” can refer to the diameter of the particle; or when the particle is substantially non-spherical, the term “size” can refer to the largest length of the particle.

The terms “coupled” or “connected” as used in this description are intended to cover both directly connected or connected through one or more intermediate means, unless otherwise stated.

The term “associated with”, used herein when referring to two elements refers to a broad relationship between the two elements. The relationship includes, but is not limited to a physical, a chemical or a biological relationship. For example, when element A is associated with element B, elements A and B may be directly or indirectly attached to each other or element A may contain element B or vice versa.

The term “adjacent” used herein when referring to two elements refers to one element being in close proximity to another element and may be but is not limited to the elements contacting each other or may further include the elements being separated by one or more further elements disposed therebetween.

The term “and/or”, e.g., “X and/or Y” is understood to mean either “X and Y” or “X or Y” and should be taken to provide explicit support for both meanings or for either meaning.

Further, in the description herein, the word “substantially” whenever used is understood to include, but not restricted to, “entirely” or “completely” and the like. In addition, terms such as “comprising”, “comprise”, and the like whenever used, are intended to be non-restricting descriptive language in that they broadly include elements/components recited after such terms, in addition to other components not explicitly recited. For example, when “comprising” is used, reference to a “one” feature is also intended to be a reference to “at least one” of that feature. Terms such as “consisting”, “consist”, and the like, may in the appropriate context, be considered as a subset of terms such as “comprising”, “comprise”, and the like. Therefore, in embodiments disclosed herein using the terms such as “comprising”, “comprise”, and the like, it will be appreciated that these embodiments provide teaching for corresponding embodiments using terms such as “consisting”, “consist”, and the like. Further, terms such as “about”, “approximately” and the like whenever used, typically means a reasonable variation, for example a variation of +/−5% of the disclosed value, or a variance of 4% of the disclosed value, or a variance of 3% of the disclosed value, a variance of 2% of the disclosed value or a variance of 1% of the disclosed value.

Furthermore, in the description herein, certain values may be disclosed in a range. The values showing the end points of a range are intended to illustrate a preferred range. Whenever a range has been described, it is intended that the range covers and teaches all possible sub-ranges as well as individual numerical values within that range. That is, the end points of a range should not be interpreted as inflexible limitations. For example, a description of a range of 1% to 5% is intended to have specifically disclosed sub-ranges 1% to 2%, 1% to 3%, 1% to 4%, 2% to 3% etc., as well as individually, values within that range such as 1%, 2%, 3%, 4% and 5%. It is to be appreciated that the individual numerical values within the range also include integers, fractions and decimals. Furthermore, whenever a range has been described, it is also intended that the range covers and teaches values of up to 2 additional decimal places or significant figures (where appropriate) from the shown numerical end points. For example, a description of a range of 1% to 5% is intended to have specifically disclosed the ranges 1.00% to 5.00% and also 1.0% to 5.0% and all their intermediate values (such as 1.01%, 1.02% . . . 4.98%, 4.99%, 5.00% and 1.1%, 1.2% . . . 4.8%, 4.9%, 5.0% etc.,) spanning the ranges. The intention of the above specific disclosure is applicable to any depth/breadth of a range.

Additionally, when describing some embodiments, the disclosure may have disclosed a method and/or process as a particular sequence of steps. However, unless otherwise required, it will be appreciated that the method or process should not be limited to the particular sequence of steps disclosed. Other sequences of steps may be possible. The particular order of the steps disclosed herein should not be construed as undue limitations. Unless otherwise required, a method and/or process disclosed herein should not be limited to the steps being carried out in the order written. The sequence of steps may be varied and still remain within the scope of the disclosure.

Furthermore, it will be appreciated that while the present disclosure provides embodiments having one or more of the features/characteristics discussed herein, one or more of these features/characteristics may also be disclaimed in other alternative embodiments and the present disclosure provides support for such disclaimers and these associated alternative embodiments.

### DESCRIPTION OF EMBODIMENTS

Exemplary, non-limiting embodiments of methods of profiling microbiome/microbiotic compositions are described herein. In the first aspect, there is provided a method of profiling a microbiotic composition of a sample comprising the nucleic acid molecules of the microbiotic composition, the method comprising:
a) partitioning the sample into a sufficient number of partitions such that at least a portion of the partitions comprises no more than one nucleic acid molecule of the microbiotic composition;
b) contacting the partitions of the sample to a plurality of mismatch-tolerant probes that are capable of binding to the nucleic acid molecules or parts thereof under suitable conditions;
c) determining signals generated by each of the plurality of mismatch-tolerant probes in each partition; and
d) establishing the microbiotic composition in the sample based on the signals.

Also disclosed is a method of profiling a microbiome composition in a sample comprising bacterial nucleic acid molecules, the method comprising:
a) partitioning the sample into a sufficient number of partitions such that at least a portion of the partitions comprises no more than one bacterial nucleic acid molecule;
b) contacting the partitions of the sample to a plurality of mismatch-tolerant probes that are capable of binding to the bacterial nucleic acid molecules or parts thereof under suitable conditions;
c) determining signals generated by each of the plurality of mismatch-tolerant probes in each partition; and
d) establishing the microbiome composition in the sample based on the signals.

In some examples, the method further comprises subjecting the partitions of the sample to an amplification reaction in the presence of a plurality of mismatch-tolerant probes that are capable of binding to the bacterial nucleic acid molecules or parts thereof under suitable conditions. For example, the contacting of the partitions of the sample to the plurality of mismatch-tolerant probes may occur in an amplification reaction.

As nucleic acid molecule may be single-stranded or double-stranded, in some examples, the bacterial nucleic acid molecule as described herein may be single-stranded or double-stranded.

As exemplified in the Experimental Section below, individual bacterial DNA is confined in separate partitions.

In some examples, the plurality of mismatch-tolerant probes is configured to collectively generate different signal intensity profiles for different groups of nucleic acid molecules.

In some examples, the plurality of mismatch-tolerant probes is configured to collectively generate different signal intensity profiles for different groups of bacterial nucleic acid molecules.

In some examples, the plurality of mismatch-tolerant probes is a plurality of sloppy molecular beacon (SMB), which are mismatch-tolerant probes that hybridises with and generate detectable signal for more than one target sequence at a detection temperature in an assay, and various hybrids so form will have different melting points. Examples of such probes are hairpin or linear probes with an internal fluorescent moiety whose level of fluorescence increases upon hybridisation to one or another target strand.

In some examples, the mismatch-tolerant probes may be dual-labelled probes, which may contain a target binding sequence flanked by a pair of arms complementary to one another. They can be DNA, RNA, or PNA, or a combination of all three nucleic acids.

In some examples, the mismatch-tolerant probes may contain modified nucleotides and/or modified internucleotide linkages.

In some examples, the mismatch-tolerant probes can have a first fluorophore on one arm and a second fluorophore on the other arm, wherein the absorption spectrum of the second fluorophore substantially overlaps the emission spectrum of the first fluorophore. In some examples, the mismatch-tolerant probes may be a hairpin probe that have a fluorophore on one arm and a quencher on the other arm such that the probes are dark when free in solution. In some examples, the mismatch-tolerant probes can also be wavelength-shifting molecular beacon probes with, for example, multiple flurophores on one arm that interact by fluorescence resonance energy transfer (FRET), and a quencher on the other arm.

In some examples, the target binding sequences can be, for example, 10 to 80, or 20 to 50, or 20, or 21, or 22, or 23, or 24, or 25, or 26, or 27, or 28, or 29, or 30, or 31, or 32, or 33, or 34, or 35, or 36, or 37, or 38, or 39, or 40, or 41, or 42, or 43, or 44, or 45, or 46, or 47, or 48, or 49, or 50 nucleotides in length, and the hybridizing arms can be 2 to 10 or 2 to 6 (e.g. 2, 3, 4, 5, or 6) nucleotides in length. In some examples, as known in the art, molecular beacon probes may be tethered to primers.

The mismatch-tolerant probes as described herein is capable of producing a detectable signal in a homogeneous assay, that is, without having to separate probes hybridized to target from unbound probes. In particular, as shown in the Experimental Section below, the mismatched-tolerant probes as described herein can generate specific melting temperature (Tm values) for DNA sequences that differed by as little as one nucleotide. This is made possible by virtue of their ability to bind to more than one variant of a given target sequence. That is, the probes as disclosed herein can be used in assays to detect the presence of one variant of a nucleic acid sequence segment of interest from among a number of possible variants or even to detect the presence of two or more variants. The probes can therefore be used in combinations of two or more in the same assay. Due to the differences in target binding sequence, the probes relative avidities for different variants are different. For example, a first probe may bind strongly to a first target sequence, moderately to a first allele of the first target sequence, weakly to a second allele of the first target sequence and not at all to a third allele of the first target sequence; while a second probe may bind weakly to the first target sequence and the first variant, and moderately to the second variant and the third variant. Additional mismatch-tolerant probes will exhibit yet different binding patterns due to their different target binding sequences. Thus, fluorescence emission spectra from combinations of sloppy probes define different microbial strains or species, as well as allelic variants/mutation of genes.

As in some examples, the mismatch-tolerant probes reproducibly fluoresce with variable intensities after binding to different DNA sequences, combinations can be used in, for example, simple, rapid, and sensitive nucleic acid amplification reaction assays (e.g., PCR-based assays) that identify multiple microorganisms or variants in a single reaction container. It is understood, however, that the assays can be performed also on samples suspected of containing directly detectable amounts of unamplified target nucleic acids. This identification assay is based on analyzing the spectra of a set of partially hybridizing signalling mismatch-tolerant probes, such as mismatch-tolerant molecular beacon probes, each labeled with a fluorophore that emits light with a different wavelength optimum, to generate “signature spectra” of species-specific or variant-specific DNA sequences.

Using the probes, multiplexing can be achieved, for example, by designing a different allele-discriminating mismatch-tolerant probe for each target (for example a microbe phylum) and labeling each probe differentially. Mixtures of allele-discriminating probes, each comprising aliquots of multiple colors, extends the number of probe signatures. To that end, every molecular beacon-target hybrid with a unique melting temperature will have corresponding unique signal intensity at a defined temperature and concentration of probe and amplicon. Thus, a limited number of mismatch-tolerant probes could be used as probes to identify many different possible target sequences in a real-time PCR reaction. The probes can be added to the amplification reaction mixture before, during, or after the amplification.

In some examples, step d) further comprises classifying the Tm signature as belonging to a group of nucleic acid molecules. In some examples, step d) as described herein comprises computing a melting temperature (Tm) of each of the plurality of mismatch-tolerant probes to obtain a Tm signature for each partition based on the signals generated by each of the plurality of mismatch-tolerant probes in each partition. The computation of the melting temperature may be conducted based on methods known in the art. In some examples, computing the Tm comprises identifying an inflection point in a signal intensity generated by each of the plurality of mismatch-tolerant probes in each partition. An illustration of one example of the embodiment of the present disclosure is provided in FIG. 1a. In some examples, the melting temperature (Tm) of each of the plurality of mismatch-tolerant probes is determined using a system that is capable of calculating real-time melting curve analysis.

In some examples, step d) further comprises determining a proportion of different groups of nucleic acid molecules in the sample based on the count numbers. In some examples, step d) as described herein further comprises classifying the Tm signature as belonging to a group of bacterial nucleic acid molecules. In some examples, step d) as described herein further comprises counting the number of partitions with the same Tm signature. In some examples, step d) as described herein further comprises determining a proportion of different groups of bacterial nucleic acid molecules in the sample based on the count numbers.

In some examples, the amplification reaction comprises any suitable PCR methodology, combination of PCR methodologies, or combination of amplification techniques, such as, but is not limited to, asymmetric PCR, allele-specific PCR, assembly PCR, digital PCR, endpoint PCR, hot-start PCR, in situ PCR, intersequence-specific PCR, inverse PCR, linear after exponential PCR, ligation-mediated PCR, methylation-specific PCR, miniprimer PCR, multiplex ligation-dependent probe amplification, multiplex PCR, nested PCR, overlap-extension PCR, polymerase cycling assembly, qualitative PCR, quantitative PCR, real-time PCR, RT-PCR, single-cell PCR, solid-phase PCR, thermal asymmetric interlaced PCR, touchdown PCR, universal fast walking PCR, and the like. A suitable PCR method would be one which final product is single stranded. In some examples, the amplification reaction comprises an asymmetric polymerase chain reaction (PCR).

In some examples, wherein the asymmetric PCR uses a primer set comprising: a pair of forward and reverse primers for amplifying the bacterial nucleic acid molecules or parts thereof to produce double-stranded PCR products; and a third primer for amplifying one of the two strands of the double-stranded PCR products or a part thereof. In some examples, the asymmetric PCR uses a primer set comprising: a pair of forward and reverse primers for amplifying the bacterial nucleic acid molecules or parts thereof to produce double-stranded PCR products; and a third primer for amplifying one of the two strands of the double-stranded PCR products or a part thereof.

In some examples, the forward and reverse primers have a higher annealing temperature than the third primer. In some examples, the third primer is present at a higher concentration than the forward and reverse primers.

In some examples, the primer as used herein may include, but is not limited to, a primer that recognises the nine variable regions of a 16s ribosomal nucleic acid region of a bacteria and/or an archea. In some examples, the primer as used herein may include, but is not limited to, a primer that recognises V1, V2, V3, V4, V5, V6, V7, V8, or V9 region of a 16s ribosomal nucleic acid region of a bacteria. In some examples, the primer as used herein may include, but is not limited to, a primer that recognises the nine variable regions of a 18s ribosomal nucleic acid region of a fungus. In some examples, the primer as used herein may include, but is not limited to, a primer that recognises V1, V2, V3, V4, V5, V6, V7, V8, or V9 region of a 18s ribosomal nucleic acid region of a fungus. In some examples, the primers as used herein may include, but is not limited to, primers may include, but is not limited to, primers provided in the table 7 and/or table 8 below:

In some examples, wherein the microbiotic composition comprises a virus, such as an influenza virus, the primer as used herein may include, but is not limited to a primer that recognises a Neuramidase (NA) and/or a Hemaglutinin (HA) of a influenza virus.

TABLE 7
Primers for 16S rRNA

| | | | Forward | SEQ | Reverse | SEQ |
|---|---|---|---|---|---|---|
| V- | Forward | Reverse | sequence | ID | sequence | ID |
| region | primer | primer | (5'-3') | NO: | (5'-3') | NO: |
| V1-V2 | 27F | 338R | AGA GTT TGA | 85 | GCT GCC TCC | 92 |
| | | | TYM TGG CTC | | CGT AGG AGT | |
| | | | AG | | | |
| V1-V3 | 27F | 534R | AGA GTT TGA | 86 | ATT ACC GCG | 93 |
| | | | TYM TGG CTC | | GCT GCT GG | |
| | | | AG | | | |
| V3-V4 | 341F | 785R | CCT ACG GGN | 87 | GAC TAC HVG | 94 |
| | | | GGC WGC AG | | GGT ATC TAA | |
| | | | | | TCC | |
| V4 | 515F | 806R | GTG CCA GCM | 88 | GGA CTA CHV | 95 |
| | | | GCC GCG GTA | | GGG TWT CTA | |
| | | | A | | AT | |
| V4-V5 | 515F | 944R | GTG CCA GCM | 89 | GAA TTA AAC | 96 |
| | | | GCC GCG GTA | | CAC ATG CTC | |
| | | | A | | | |
| V6-V8 | 939F | 1378R | GAA TTG ACG | 90 | CGG TGT GTA | 97 |
| | | | GGG GCC CGC | | CAA GGC CCG | |
| | | | ACA AG | | GGA ACG | |
| V7-V9 | 1115F | 1492R | CAA CGA GCG | 91 | TAC GGY TAC | 98 |
| | | | CAA CCC T | | CTT GTT ACG | |
| | | | | | ACT T | |

TABLE 8
Primers for 18S rRNA (from Berkeley University of California, as
disclosed in CD Genomics Blog on 17 Oct. 2018, https://www.
cd-genomics.com/blog/18s-rrna-and-its-use-in-fungal-diversity-
analysis/)

| Name | Primer Sequence | Tm | SEQ ID NO |
|---|---|---|---|
| NS1 | GTAGTCATATGCTTGTCTC | 49 | 99 |
| CNS1 | GAGACAAGCATATGACTACTG | 55 | 100 |
| NS2 | GGCTGCTGGCACCAGACTTGC | 65 | 101 |
| NS3 | GCAAGTCTGGTGCCAGCAGCC | 65 | 102 |
| NS4 | CTTCCGTCAATTCCTTTAAG | {62} | 103 |
| NS5 | AACTTAAAGGAATTGACGGAAG | 55 | 104 |
| NS6 | GCATCACAGACCTGTTATTGCCTC | {72} | 105 |
| NS7 | GAGGCAATAACAGGTCTGTGATGC | {72} | 106 |
| NS8 | TCCGCAGGTTCACCTACGGA | 59 | 107 |
| TW9 | TAAGCCATGCATGTCT | | 108 |
| TW10 | GCGGTAATTCCAGCTCC | | 109 |
| TW11 | GGAGTGGAGCCTGCGGCT | | 110 |
| TW12 | AAGTCGTAACAAGGTTT | 53 | 111 |
| CTW12 | AAACCTTGTTACGACTT | 53 | 112 |
| NS17 | CATGTCTAAGTTTAAGCAA | 55 | 113 |
| NS18 | CTCATTCCAATTACAAGACC | 60 | 114 |
| NS19 | CCGGAGAAGGAGCCTGAGAAAC | 74 | 115 |
| NS20 | CGTCCCTATTAATCATTACG | 61 | 116 |
| NS21-ag | GAATAATAGAATAGGACG | 50 | 117 |
| NS21-ls | AATATACGCTATTGGAGCTGG | | 118 |
| NS22 | AATTAAGCAGACAAATCACT | 57 | 119 |
| NS23 | GACTCAACACGGGAAACTC | 64 | 120 |
| NS24 | AAACCTTGTTACGACTTTTA | 58 | 121 |
| NS25 | GTGGTAATTCTAGAGCTAATACT | | 122 |
| CNS25 | ATGTATTAGCTCTAGAATTACCAC | | 123 |
| NS26 | CTGCCCTATCAACTTTCGA | | 124 |
| CNS26 | TCGAAAGTTGATAGGGCAG | | 125 |
| VANS1 | GTCTAGTATAATCGTTATACAGG | 57 | 126 |
| MB1 | GGAGTATGGTCGCAAGGCTG | | 127 |
| CMB1 | CAGCCTTGCGACCATACTCC | | 128 |
| MB2 | GTGAGTTTCCCCGTGTTGAG | 57 | 129 |
| Basid 1 | TTGCTACATGGATAACTGTG | 49 | 130 |
| Basid 2 | CTGTTAAGACTACAACGG | | 131 |
| Basid 3 | AGAGTGTTCAAAGCAGGC | | 132 |
| Basid 4 | CTCACTAAGCCATTCAATCGG | | 133 |
| NS1.5R | TCTAGAGCTAATACATGC(T/C)G | 52 | 134 |
| NS2.8R | GGCCCTCAAATCTAAGGATT | 53 | 135 |
| CNS2.8R | AATTTGCGCGCCTGCTGCAA | 57 | 136 |
| NS3.2R | CGTATATTAAAATTGTTGAC | 45 | 137 |
| CNS3.3R | GACTACGAGCTTTTTAACGT | 51 | 138 |
| CNS3.5R | TTTCGCAGTAGTTTGTCTTA | 49 | 139 |
| NS3.6R | CAAACTACTGCGAAAGCATC | 53 | 140 |
| CNS3.6R | AATGAAGTCATCCTTGGCAG | 53 | 141 |

In some examples, the primers comprise at least one sequence selected from the group consisting of: ACTCCTACGGGAGGCAGCAG (SEQ ID NO: 1) or a sequence sharing at least about 85% sequence identity thereto; ATTACCGCGGCTGCTGG (SEQ ID NO: 2) or a sequence sharing at least about 85% sequence identity thereto; TACGGGAGGCAGCAG (SEQ ID NO: 3) or a sequence sharing at least about 85% sequence identity thereto, CTGGCACCAAGCAGAAGACGGCA (SEQ ID NO: 66) or a sequence sharing at least about 85% sequence identity thereto, ACGGCGACCACCGAGATCTACC (SEQ ID NO: 67) or a sequence sharing at least about 85% sequence identity thereto; and GGCACCAAGCAGAAGA (SEQ ID NO: 68) or a sequence sharing at least about 85% sequence identity thereto. In some examples, the primers may comprise a sequence sharing at least about 89%, or 90%, or 91%, or 92%, or 93%, or 94%, or 95%, or 96%, or 97%, or 98%, or 99% sequence identity thereto. In some examples, the primers may comprise a sequence having one, or two, or three nucleotide differences thereto.

In some examples, the plurality of mismatch-tolerant probes as disclosed herein may be configured to bind to a nucleic acid region of the microbiotic composition. In some examples, the plurality of mismatch-tolerant probes as disclosed herein may be configured to bind to a 16s and/or 18s ribosomal region of nucleic acid region of the microbiotic composition. In some examples, the plurality of mismatch-tolerant probes as disclosed herein may be configured to bind to a 16s and/or 18s ribosomal region of nucleic acid region at temperatures below their melting temperature. In some examples, wherein the microbiotic composition comprises a virus, such as an influenza virus, the plurality of mismatch-tolerant probes as disclosed herein may be configured to bind to the a Neuramidase (NA) and/or a Hemaglutinin (HA) of region of the influenza virus strain.

In some examples, the plurality of mismatch-tolerant probes may be configured to the ribosomal region of nucleic acid molecules of microbes. In some examples, the microbes may be bacteria including, but not limited to, one or more Staphylococcus aureus (coagulase negative Staphylococcus sp., Mycobacterium species, Haemophilus species, Genera Prevotella, Fusobacterium, Streptococcus, Granulicatella, Bacteroides, Porphyromonas, Treponema, Genera Veillonella, Eubacterium, Enterococcus, Catonella, Selenomonas, Actinobacteria (such as genus Arthrobacter), β-Proteobacteria (such as genus Gallionella), α-Proteobacteria, γ-Proteobacteria (such as Acinetobacter), Chlamydiae, and the like.

In some examples, the plurality of mismatch-tolerant probes is configured to bind to one or more V1, V2, V3, V4, V5, V6, V7, V8, and V9 of a 16s and/or 18s ribosomal region of nucleic acid region of the microbiotic composition. In some examples, the plurality of mismatch-tolerant probes is configured to bind to 1, 2, 3, 4, 5, 6, 7, 8, or all of the 16s and/or 18s ribosomal region of nucleic acid region of the microbiotic composition.

In some examples, the plurality of mismatch-tolerant probes is configured to bind to a V1 region of a 16s and/or 18s ribosomal nucleic acid region of the nucleic acid molecules at temperature below their melting temperature. In some examples, the mismatch-tolerant probes may be configured to bind to a V1 region of the nucleic acid molecules for bacteria including, but is not limited to, Staphylococcus aureus (coagulase negative Staphylococcus sp., Genera Prevotella, Fusobacterium, Streptococcus, Granulicatella, Bacteroides, Porphyromonas, Treponema, Actinobacteria (such as genus Arthrobacter), and the like.

In some examples, the plurality of mismatch-tolerant probes is configured to bind to a V2 region of a 16s and/or 18s ribosomal nucleic acid region of the nucleic acid molecules at temperature below their melting temperature. In some examples, the mismatch-tolerant probes may be configured to bind to a V2 region of the nucleic acid molecules for bacteria including, but is not limited to, Mycobacterium species, Genera Prevotella, Fusobacterium, Streptococcus, Granulicatella, Bacteroides, Porphyromonas, Treponema, Actinobacteria (such as genus Arthrobacter), and the like.

In some examples, the plurality of mismatch-tolerant probes is configured to bind to a V3 region of a 16s and/or 18s ribosomal nucleic acid region of the nucleic acid molecules at temperature below their melting temperature. In some examples, the mismatch-tolerant probes may be configured to bind to a V3 region of the nucleic acid molecules for bacteria including, but is not limited to, Haemophilus species, Genera Prevotella, Fusobacterium, Streptococcus, Granulicatella, Bacteroides, Porphyromonas, Treponema, Actinobacteria (such as genus Arthrobacter), β-Proteobacteria (such as genus Gallionella), α-Proteobacteria, and the like.

In some examples, the plurality of mismatch-tolerant probes is configured to bind to a V3, V4, or V5 region of a 16s and/or 18s ribosomal nucleic acid region of the nucleic acid molecules at temperature below their melting temperature. In some examples, the mismatch-tolerant probes may be configured to bind to a V3, V4, or V5 region of the nucleic acid molecules for bacteria including, but is not limited to, Actinobacteria (such as genus Arthrobacter), and the like.

In some examples, the plurality of mismatch-tolerant probes is configured to bind to a V7, V8, or V9 region of a 16s and/or 18s ribosomal nucleic acid region of the nucleic acid molecules at temperature below their melting temperature. In some examples, the mismatch-tolerant probes may be configured to bind to a V7, V8, or V9 region of the nucleic acid molecules for bacteria including, but is not limited to, Genera Veillonella, Streptococcus, Eubacterium, Enterococcus, Treponema, Catonella, Selenomonas, β-Proteobacteria (such as genus Gallionella), α-Proteobacteria, γ-Proteobacteria (such as Acinetobacter), and the like.

In some examples, the plurality of mismatch-tolerant probes is configured to bind to a V8 region of a 16s and/or 18s ribosomal nucleic acid region of the nucleic acid molecules at temperature below their melting temperature. In some examples, the mismatch-tolerant probes may be configured to bind to a V8 region of the nucleic acid molecules for bacteria including, but is not limited to, β-Proteobacteria (such as genus Gallionella), γ-Proteobacteria (such as Acinetobacter), Chlamydiae, and the like.

In some examples, the plurality of mismatch-tolerant probes is configured to bind to a V6, V7, or V8 region of a 16s and/or 18s ribosomal nucleic acid region of the nucleic acid molecules at temperature below their melting temperature. In some examples, the mismatch-tolerant probes may be configured to bind to a V6, V7, or V8 region of the nucleic acid molecules for bacteria including, but is not limited to, 3-Proteobacteria, Chlamydiae, and the like.

In some examples, the plurality of mismatch-tolerant probes is configured to bind to a V3 region of a 16s and/or 18s ribosomal nucleic acid region of the nucleic acid molecules at temperatures below their melting temperatures. In some examples, the plurality of mismatch-tolerant probes is configured to bind to a region of the bacterial nucleic acid molecules at temperatures below their melting temperatures. In some examples, the plurality of mismatch-tolerant probes is configured to bind to a 16s ribosomal nucleic acid region of the bacterial nucleic acid molecules. In some examples, the plurality of mismatch-tolerant probes is configured to bind to a V3 region of a 16s ribosomal nucleic acid region of the bacterial nucleic acid molecules. In some examples, the plurality of mismatch-tolerant probes is configured to bind to a V3 region of a 16s ribosomal nucleic acid region of the bacterial nucleic acid molecules at temperatures below their melting temperatures.

Without wishing to be bound by theory, the inventor of the present disclosure developed a bioinformatic pipeline to design an optimal set of 40 nucleotide-long sloppy molecular beacon (SMB) probes (i.e. mismatch-tolerant probes) for the determination of the phylum of specific 16S and/or 18s rRNA gene sequences. Using a sliding-window approach, the inventor found that the user can determined probe sequences at each position that would have the greatest affinity and least intra-phylum variability with a target phylum. The user would then chose a probe sequence among these sequences that can best distinguish the target phylum from non-target phylum. Using this approach, SMB probes can be identified, that when used simultaneously, allow for accurate determination of the phylum.

Therefore, in some examples, the plurality of mismatch-tolerant probes is configured to have different Tm signatures for different groups of nucleic acid molecules from the microbiotic composition. For example, the plurality of mismatch-tolerant probe for a first microbiotic phylum may be designed to have a highest Tm signature, the plurality of mismatch-tolerant probe for a second microbiotic phylum may be designed to have a mid-range Tm signature, the plurality of mismatch-tolerant probe for a third microbiotic phylum may be designed to have a low Tm signature, and the plurality of mismatch-tolerant probe for a fourth microbiotic phylum may be designed to have the lowest Tm signature.

In some examples, the plurality of mismatch-tolerant probes is configured to have different Tm signatures for different groups of bacterial nucleic acid molecules. For example, the plurality of mismatch-tolerant probe for a first bacterial phylum may be designed to have a highest Tm signature, the plurality of mismatch-tolerant probe for a second bacterial phylum may be designed to have a mid-range Tm signature, the plurality of mismatch-tolerant probe for a third bacterial phylum may be designed to have a low Tm signature, and the plurality of mismatch-tolerant probe for a fourth bacterial phylum may be designed to have the lowest Tm signature.

In some examples, the plurality of mismatch-tolerant probes is configured to have different Tm signatures for nucleic acid molecules from different phyla of microbiotic composition.

In some examples, the plurality of mismatch-tolerant probes is configured to have different Tm signatures for different phyla of bacterial nucleic acid molecules.

In some examples, the microbiotic composition comprises one or more microbes comprising bacteria, fungi, virus, archaea, or combinations thereof.

In some examples, the microbiotic composition may be one or more microbes comprising bacteria, fungi, and/or combination thereof.

In some examples, the nucleic acid of the microbiotic composition may comprise one or more microbiotic nucleic acid from bacteria, fungi, and/or combination thereof.

In some examples, the microbiotic composition may include, but is not limited to, microbes from
one or more bacteria from the genus of Acetobacter, Acinetobacter, Actinomyces, Agrobacterium spp., Azorhizobium, Azotobacter, Anaplasma spp., Bacillus spp., Bacteroides spp., Bartonella spp., Bordetella spp., Borrelia, Brucella spp., Burkholderia spp., Calymmatobacterium, Campylobacter, Chlamydia spp., Chlamydophila spp., Clostridium spp., Corynebacterium spp., Coxiella, Ehrlichia, Enterobacter, Enterococcus spp., Escherichia, Francisella, Fusobacterium, Gardnerella, Haemophilus spp., Helicobacter, Klebsiella, Lactobacillus spp., Lactococcus, Legionella, Listeria, Methanobacterium extroquens, Microbacterium multiforme, Micrococcus luteus, Moraxella catarrhalis, Mycobacterium spp., Mycoplasma spp., Neisseria spp., Pasteurella spp., Peptostreptococcus, Porphyromonas, Pseudomonas, Rhizobium, Rickettsia spp., Rochalimaea spp., Rothia, Salmonella spp., Serratia, Shigella, Staphylococcus spp., Stenotrophomonas, Streptococcus spp., Treponema spp., Vibrio spp., Wolbachia, and Yersinia spp, and/or one or more fungus from the genus Absidia, Ajellomyces, Arthroderma, Aspergillus, Blastomyces, Candida, Cladophialophora, Coccidioides, Cryptococcus, Cunninghamella, Epidermophyton, Exophiala, Filobasidiella, Fonsecaea, Fusarium, Geotrichum, Histoplasma, Hortaea, Issatschenkia, Madurella, Malassezia, Microsporum, Microsporidia, Mucor, Nectria, Paecilomyces, Paracoccidioides, Penicillium, Pichia, Pneumocystis, Pseudallescheria, Rhizopus, Rhodotorula, Scedosporium, Schizophyllum, Sporothrix, Trichophyton, and Trichosporon,
or combinations thereof.

In some examples, the microbiotic composition comprises microbes from 1 to 60 bacteria genus as disclosed herein, or 1 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 bacterial genus as disclosed herein. In some examples, the microbiotic composition comprises microbes from 1 to 60 fungal genus as disclosed herein, or 1 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, or 38, fungal genus as disclosed herein.

In some examples, the microbiotic nucleic acid may be from a bacteria. In some examples, the bacteria may be Gram-positive or Gram-negative bacteria. Thus, bacteria may be of genus including, but not limited to Acetobacter, Acinetobacter, Actinomyces, Agrobacterium spp., Azorhizobium, Azotobacter, Anaplasma spp., Bacillus spp., Bacteroides spp., Bartonella spp., Bordetella spp., Borrelia, Brucella spp., Burkholderia spp., Calymmatobacterium, Campylobacter, Chlamydia spp., Chlamydophila spp., Clostridium spp., Corynebacterium spp., Coxiella, Ehrlichia, Enterobacter, Enterococcus spp., Escherichia, Francisella, Fusobacterium, Gardnerella, Haemophilus spp., Helicobacter, Klebsiella, Lactobacillus spp., Lactococcus, Legionella, Listeria, Methanobacterium extroquens, Microbacterium multiforme, Micrococcus luteus, Moraxella catarrhalis, Mycobacterium spp., Mycoplasma spp., Neisseria spp., Pasteurella spp., Peptostreptococcus, Porphyromonas, Pseudomonas, Rhizobium, Rickettsia spp., Rochalimaea spp., Rothia, Salmonella spp., Serratia, Shigella, Staphylococcus spp., Stenotrophomonas, Streptococcus spp., Treponema spp., Vibrio spp., Wolbachia, and Yersinia spp. In one example, the bacteria include, but are not limited to Acetobacter aurantius, Acinetobacter baumannii, Actinomyces Israelii, Agrobacterium radiobacter, Agrobacterium tumefaciens, Azorhizobium caulinodans, Azotobacter vinelandii, Anaplasma phagocytophilum, Anaplasma marginale, Bacillus anthracis, Bacillus brevis, Bacillus cereus, Bacillus fusiformis, Bacillus licheniformis, Bacillus megaterium, Bacillus mycoides, Bacillus stearothermophilus, Bacillus subtilis, Bacteroides fragilis, Bacteroides gingivalis, Bacteroides melaminogenicus (Prevotella melaminogenica), Bartonella henselae, Bartonella quintana, Bordetella bronchiseptica, Bordetella pertussis, Borrelia burgdorferi, Brucella abortus, Brucella melitensis, Brucella suis, Burkholderia mallei, Burkholderia pseudomallei, Burkholderia cepacia complex, Burkholderia cenocepacia, Calymmatobacterium granulomatis, Campylobacter coli, Campylobacter fetus, Campylobacter jejuni, Campylobacter pylori, Chlamydia trachomatis, Chlamydophila. (such as C. pneumoniae, Chlamydophila psittaci, Clostridium botulinum, Clostridium difficile, Clostridium perfringens, Clostridium tetani), Corynebacterium diphtheriae, Corynebacterium fusiforme, Coxiella bumetii, Ehrlichia chaffeensis, Enterobacter cloacae, Enterococcus avium, Enterococcus durans, Enterococcus faecalis, Enterococcus faecium, Enterococcus galllinarum, Enterobacter gergoviae (now known as Pluralibacter gergoviae), Enterococcus maloratus, Escherichia coli, Francisella tularensis, Fusobacterium nucleatum, Gardnerella vaginalis, Haemophilus ducreyi, Haemophilus influenzae, Haemophilus parainfluenzae, Haemophilus pertussis, Haemophilus vaginalis, Helicobacter pylori, Klebsiella pneumoniae, Lactobacillus acidophilus, Lactobacillus casei, Lactococcus lactis, Legionella pneumophila, Listeria monocytogenes, Methanobacterium extroquens, Microbacterium multiforme, Micrococcus luteus, Moraxella catarrhalis, Mycobacterium avium, Mycobacterium bovis, Mycobacterium diphtheriae, Mycobacterium intracellulare, Mycobacterium leprae, Mycobacterium lepraemurium, Mycobacterium phlei, Mycobacterium smegmatis, Mycobacterium tuberculosis, Mycoplasma fermentans, Mycoplasma genitalium, Mycoplasma hominis, Mycoplasma penetrans, Mycoplasma pneumoniae, Neisseria gonorrhoeae, Neisseria meningitidis, Pasteurella multocida, Pasteurella tularensis Peptostreptococcus, Porphyromonas gingivalis, Pseudomonas aeruginosa, Rhizobium Radiobacter, Rickettsia prowazekii, Rickettsia psittaci, Rickettsia quintana, Rickettsia rickettsii, Rickettsia trachomae, Rochalimaea henselae, Rochalimaea quintana, Rothia dentocariosa, Salmonella enteritidis, Salmonella typhi, Salmonella typhimurium, Serratia marcescens, Shigella dysenteriae, Staphylococcus aureus, Staphylococcus epidermidis, Stenotrophomonas maltophilia, Streptococcus agalactiae, Streptococcus. avium, Streptococcus bovis, Streptococcus cricetus, Streptococcus faceium, Streptococcus faecalis, Streptococcus ferus, Streptococcus gallinarum, Streptococcus lactis, Streptococcus mitior, Streptococcus mitis, Streptococcus mutans, Streptococcus oralis, Streptococcus pneumoniae, Streptococcus pyogenes, Streptococcus rattus, Streptococcus salivarius, Streptococcus sanguis, Streptococcus sobrinus, Treponema pallidum, Treponema denticola, Vibrio cholerae, Vibrio comma, Vibrio parahaemolyticus, Vibrio vulnificus, Wolbachia, Yersinia enterocolitica, Yersinia pestis and Yersinia pseudotuberculosis.

In some examples, the microbiotic nucleic acid may be from a fungi. In some examples, fungi includes, but is not limited to, references to organisms of the following genus Absidia, Ajellomyces, Arthroderma, Aspergillus, Blastomyces, Candida, Cladophialophora, Coccidioides, Cryptococcus, Cunninghamella, Epidermophyton, Exophiala, Filobasidiella, Fonsecaea, Fusarium, Geotrichum, Histoplasma, Hortaea, Issatschenkia, Madurella, Malassezia, Microsporum, Microsporidia, Mucor, Nectria, Paecilomyces, Paracoccidioides, Penicillium, Pichia, Pneumocystis, Pseudallescheria, Rhizopus, Rhodotorula, Scedosporium, Schizophyllum, Sporothrix, Trichophyton, and Trichosporon. In one example, the fungus may include, but is not limited to Absidia corymbifera, Ajellomyces capsulatus, Ajellomyces dermatitidis, Arthroderma benhamiae, Arthroderma fulvum, Arthroderma gypseum, Arthroderma incurvatum, Arthroderma otae and Arthroderma vanbreuseghemii, Aspergillus flavus, Aspergillus fumigatus and Aspergillus niger, Blastomyces dermatitidis, Candida albicans, Candida glabrata, Candida guilliermondii, Candida krusei, Candida parapsilosis, Candida tropicalis and Candida pelliculosa, Cladophialophora carrionii, Coccidioides immitis and Coccidioides posadasii, Cryptococcus neoformans, Cunninghamella Sp, Epidermophyton floccosum, Exophiala dermatitidis, Filobasidiella neoformans, Fonsecaea pedrosoi, Fusarium solani, Geotrichum candidum, Histoplasma capsulatum, Hortaea werneckii, Issatschenkia orientalis, Madurella grisae, Malassezia furfur, Malassezia globosa, Malassezia obtusa, Malassezia pachydermatis, Malassezia restricta, Malassezia slooffiae, Malassezia sympodialis, Microsporum canis, Microsporum fulvum, Microsporum gypseum, Microsporidia, Mucor circinelloides, Nectria haematococca, Paecilomyces variotii, Paracoccidioides brasiliensis, Penicillium marneffei, Pichia anomala, Pichia guilliermondii, Pneumocystis firoveci, Pneumocystis carinii, Pseudallescheria boydii, Rhizopus oryzae, Rhodotorula rubra, Scedosporium apiospermum, Schizophyllum commune, Sporothnx schenckii, Trichophyton mentagrophytes, Trichophyton rubrum, Trichophyton verrucosum and Trichophyton violaceum, and Trichosporon asahii, Trichosporon cutaneum, Trichosporon inkin and Trichosporon mucoides.

In some examples, the microbiotic composition comprises combinations of bacteria and/or fungus thereof.

In some examples, the sample may be a medical sample and/or a non-medical sample.

In some examples, the sample may be a medical sample and/or an non-medical (e.g. industrial or commercial) sample. The sample may be a fluid (such as a liquid or air) or a solid (including semi solids). In some examples, the sample may be a plane or a surface of interest. Therefore, the present disclosure may be applied to any surface for plane whose mechanical structure is compatible with the adherence of a microbiotic composition such as bacteria, fungi (including yeast), virus, and the like. In some examples, the microbes is a bacteria and/or fungi. In some examples, the microbiotic composition may comprise of microbes found in an environment, a product, a person, an organ, or a tissue.

In the context of the methods as disclosed herein, the terminology “sample” encompass the inner and outer aspects of various composition, instruments and devices, both disposable and non-disposable, medical and non-medical.

In some examples, the sample is a sample found in one or more selected from the group consisting of food industries, consumer products, agriculture, and laboratory.

Examples of non-medical sample include a sample found in food industries (such as in food preparation, preservatives, additives, and the like), consumer products (such as in shampoo, cream, moisturizer, hand sanitizer, soaps, and the like), agriculture (such as in soil composition, water-bodies fertilizer composition, additives, meat or poultry samples, samples from sources of potential contamination or environment of interest (environmental), and the like), or laboratory (such as experimental animals including rodents (rats, mice, and the like), rabbits, non-human primates, and the like).

Examples of medical samples include the entire spectrum of medical devices. Such “samples” may include the inner and outer aspects of various instruments and devices, whether disposable or intended for repeated uses.

In some examples, the sample may be a medical sample such as a biological sample. In some examples, the sample may be a tissue or organ that comprises microbiotic/microbial/microbes component. In some examples, the microbiotic composition may be desirable or undesirable. Desirable microbiotic component may be a harmless and/or health supporting microbiome. Undesirable microbiotic component may be a microbiotic composition that are harmful to the host and/or causes a disease/disorder in the host. For example a desirable microbiome is a heathy gut flora (including but not limited to gut flora comprising Lactobacillus, Escherichia coli, Bifido-bacterium, and the like) and an undesirable microbiome is a damaged gut flora (including but not limited to Staphylococcus aureus, Clostridium perfringens, Salmonella, and the like).

In some examples, the sample is a biological sample. In some examples, the sample is a biological sample comprising stool, whole blood, serum, plasma, tears, saliva, nasal fluid, sputum, ear fluid, genital fluid, breast fluid, milk, colostrum, placental fluid, amniotic fluid, perspirate, synovial fluid, ascites fluid, cerebrospinal fluid, bile, gastric fluid, aqueous humor, vitreous humor, gastrointestinal fluid, exudate, transudate, pleural fluid, pericardial fluid, semen, upper airway fluid, peritoneal fluid, fluid harvested from a site of an immune response, fluid harvested from a pooled collection site, bronchial lavage, urine, a biopsy material of a gastrointestinal tract, a biopsy material of a respiratory tract, a biopsy material of a musculoskeletal system, a biopsy material of a reproductive system, a biopsy material of a nervous system, a biopsy material of a renal/urinary system, a biopsy material of an immune system, a biopsy material of a endocrine system, a biopsy material of an integumentary system, a biopsy material of a circulatory system/cardiovascular system.

In some examples, the biological sample may include, but is not limited to samples derived from or comprising stool, whole blood, serum, plasma, tears, saliva, nasal fluid, sputum, ear fluid, genital fluid, breast fluid, milk, colostrum, placental fluid, amniotic fluid, perspirate, synovial fluid, ascites fluid, cerebrospinal fluid, bile, gastric fluid, aqueous humor, vitreous humor, gastrointestinal fluid, exudate, transudate, pleural fluid, pericardial fluid, semen, upper airway fluid, peritoneal fluid, fluid harvested from a site of an immune response, fluid harvested from a pooled collection site, bronchial lavage, urine, biopsy material from all suitable organs, such as, but not limited to the gastrointestinal tract (such as, but not limited to, mouth, esophagus, stomach, small intestine, large intestine, colon, rectum, and the like), the respiratory tract (such as, but not limited to, nose, mouth, nasal passage/sinuses, pharynx, rachea, bronchial tubes, lungs, and the like), the musculoskeletal system (such as, but not limited to bones, muscles, tendons, ligaments, soft tissues, and the like), the reproductive system (such as, but not limited to the female reproductive system including ovaries, fallopian tubes, uterus, cervix, vagina, and vulva; the male reproductive system including testes, epididymis, vas deferens, ejaculatory ducts, urethra, and penis), the nervous system (such as the central nervous system including the brain and spinal cord; and the peripheral nervous system), renal/urinary system (such as kidneys, bladder, and the urinary tract), immune system (such as the lymphatic vessels, spleen, and the like), endocrine system (such as the pineal gland, pituitary gland, parathyroid gland, thyroid gland, adrenal gland, pancreas, ovary, testis), integumentary system such as exocrine glands (including sweat, salivary, mammary, ceruminous, lacrimal, sebaceous, protrate, mucous, and the like), skin, hair, nails, and the like, the circulatory system/cardiovascular system (including heart, arteries, veins, and the like), and other tissues in the body of a human or a non-human animal that comprises microbiome. In some example, the sample may be a skin swab/biopsy, a colon biopsy, a colorectal biopsy, and the like.

In some examples, the microbiotic composition may be from a sample of the conjunctiva, the outer ear, the stomach, the skin, the urethra, the vagina, the nose, the mouth and/or oropharynx, the small intestine, the large intestines, and the like.

In some examples, the microbiotic composition found in the outer ear sample may include, but is not limited to, one or more of Staphylococci (coagulase-negative), Diphtheroids, Pseudomonas spp., Enterobacteriaceae (Peptostreptococcus), and the like.

In some examples, the microbiotic composition found in the stomach might include, but is not limited to, one or more of Streptococcus, Staphylococcus, Lactobaccilus, Peptostreptococcus, and the like.

In some examples, the microbiotic composition found in the skin may include, but is not limited to, one or more of Staphylococci (coagulase-negative), Diphtheroids (including Propionibacterium acnes), Staphylococcus aureus, Streptococci (various species), Bacillus spp., Malassezia furfur, Candida spp., Mycobacterium spp., and the like.

In some examples, the microbiotic composition found in the urethra may include, but is not limited to, one or more of Staphylococci (coagulase-negative), Diphtheroids, Streptococci (various species), Mycobacterium spp., Bacteroides spp., Fusobacterium spp., Peptostreptococcus spp., and the like.

In some examples, the microbiotic composition found in the vagina may include, but is not limited to, one or more of Lactobacillus spp., Peptostreptococcus pp., Diphtheroids, Streptococci (various), Clostridium spp., Bacteroides spp., Candida spp., Gardnerella vaginalis, and the like.

In some examples, the microbiotic composition found in the nose may include, but is not limited to, one or more of Staphylococci (coagulase-negative), Viridans streptococci, Staphylococcus aureus, Neisseria spp., Haemophilus spp., Streptococcus pneumoniae, and the like.

In some examples, the microbiotic composition found in the mouth and oropharynx may include, but is not limited to Viridians streptococci, Staphylococci (coagulase-negative), Veillonella spp., Fusobacterium spp., Treponema spp., Porphyromonas spp, Prevotella spp., Neissera spp., Branhamella catarrhalis, Streptococcus pneumoniae, Streptococci (beta-hemolytic; not group A), Candida spp., Heamophilus spp., Diphtheroids, Actinomyces spp., Ekenella corrodens, Staphylococcus aureus, and the like.

In some examples, the microbiotic composition found in the small intestine may include, but is not limited to Lactobacillus spp., Bacteroides spp., Clostridium spp., Mycobacterium spp., Enterococci, Enterobacteriaceae, and the like.

In some examples, the microbiotic composition found in the large intestine may include, but is not limited to Bacteroides spp., Fusobacterium spp., Clostridium spp., Peptostreptococcus spp., Escherichia coli, Klebsiella spp., Proteus spp., Lactobacillus spp., Enterococci, Streptococci (various species), Pseudomonas spp., Acinetobacter spp., Staphylococci (coagulase-negative), Staphylococcus aureus, Mycobacterium spp., Actinomyces spp., and the like.

In some examples, the sample may be a gut biopsy, fecal material, saliva, buccal swab, oral swab, nasal swab/biopsy, skin biopsy/swab, vagina swab, urine (urinary tract swab/biopsy), blood, wound biopsy/swab, environmental swab, water-bodies, soil, consumer product, and animals of interest. In some examples, the bacteria may be selected from the group consisting of: Firmicutes, Bacteriodetes, Actinobacteria, Proteobacteria, Fusobacteria and Verrucomicrobia.

In some examples, the plurality of mismatch-tolerant probes is configured to have different Tm signatures for different species of bacterial nucleic acid molecules.

In some examples, the plurality of mismatch-tolerant probes is configured to have different Tm signatures for different phyla of bacterial nucleic acid molecules, optionally selected from the group consisting of: Firmicutes, Bacteriodetes, Actinobacteria, Proteobacteria, Fusobacteria, and Verrucomicrobia. In some examples, the plurality of mismatch-tolerant probes is configured to have different Tm signatures for different species of bacteria, such as, but not limited to, Mycobacterium aurum, Mycobacterium branderi, Mycobacterium engbaekii, Mycobacterium senegalense, Mycobacterium shimoidei, Mycobacterium tokaiense, Mycobacterium triviale, Mycobacterium asiaticum, Mycobacterium avium, Mycobacterium celatum, Mycobacterium chubuense, Mycobacterium intracellulare, Mycobacterium kansasii, Mycobacterium szulgai, Mycobacterium terrae, Mycobacterium tuberculosis, and the like.

In some examples, the method as disclosed herein detects bacterial nucleic acid molecules from various phyla of bacteria, such as, but not limited to Firmicutes, Bacteriodetes, Actinobacteria, Proteobacteria, Fusobacteria, and Verrucomicrobia. In some examples, the plurality of mismatch-tolerant probes as described herein binds to (or capable of hybridizing to) at least one target bacterial nucleic acid sequence selected from the group consisting of SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, or fragments thereof, or a sequence sharing at least about 85% sequence identity thereto. In some examples, the target bacterial nucleic acid sequence may comprise a sequence sharing at least about 89%, or 90%, or 91%, or 92%, or 93%, or 94%, or In some examples, the plurality of mismatch-tolerant probes is configured to have different Tm signatures for different phyla of bacterial nucleic acid molecules, optionally selected from the group consisting of: Firmicutes, Bacteriodetes, Actinobacteria, Proteobacteria, Fusobacteria, and Verrucomicrobia. In some examples, the plurality of mismatch-tolerant probes is configured to have different Tm signatures for different species of bacteria, such as, but not limited to, Mycobacterium aurum, Mycobacterium branderi, Mycobacterium engbaekii, Mycobacterium senegalense, Mycobacterium shimoidei, Mycobacterium tokaiense, Mycobacterium triviale, Mycobacterium asiaticum, Mycobacterium avium, Mycobacterium celatum, Mycobacterium chubuense, Mycobacterium intracellulare, Mycobacterium kansasii, Mycobacterium szulgai, Mycobacterium terrae, Mycobacterium tuberculosis, and the like.

In some examples, the method as disclosed herein detects bacterial nucleic acid molecules from various phyla of bacteria, such as, but not limited to Firmicutes, Bacteriodetes, Actinobacteria, Proteobacteria, Fusobacteria, and Verrucomicrobia. In some examples, the plurality of mismatch-tolerant probes as described herein binds to (or capable of hybridizing to) at least one target bacterial nucleic acid sequence selected from the group consisting of SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, or fragments thereof, or a sequence sharing at least about 85% sequence identity thereto. In some examples, the target bacterial nucleic acid sequence may comprise a sequence sharing at least about 89%, or 90%, or 91%, or 92%, or 93%, or 94%, or 95%, or 96%, or 97%, or 98%, or 99% sequence identity thereto. In some examples, the bacterial nucleic acid sequence may comprise a sequence having one, or two, or three nucleotide differences thereto.

In some examples, the plurality of mismatch-tolerant probes as described herein binds to (or capable of hybridizing to) the nucleic acid component of target bacteria (such as an actinobacterium). In some examples, the actinobacterium comprises at least one sequence selected from the group consisting of SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, or fragments thereof, or a sequence sharing at least about 85% sequence identity thereto. In some examples, the nucleic acid component of target bacteria (such as an actinobacterium) may comprise a sequence sharing at least about 89%, or 90%, or 91%, or 92%, or 93%, or 94%, or 95%, or 96%, or 97%, or 98%, or 99% sequence identity thereto. In some examples, the nucleic acid component of an actinobacterium may comprise a sequence having one, or two, or three nucleotide differences thereto.

In some examples, the plurality of mismatch-tolerant probes as described herein binds to (or capable of hybridizing to) the nucleic acid component of target bacteria (such as firmicutes). In some examples, the target bacteria (such as firmicutes) comprises at least one sequence selected from the group consisting of SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, or fragments thereof, or a sequence sharing at least about 85% sequence identity thereto. In some examples, the nucleic acid component of target bacteria (such as firmicutes) may comprise a sequence sharing at least about 89%, or 90%, or 91%, or 92%, or 93%, or 94%, or 95%, or 96%, or 97%, or 98%, or 99% sequence identity thereto. In some examples, the nucleic acid component of firmicutes may comprise a sequence having one, or two, or three nucleotide differences thereto.

In some examples, the plurality of mismatch-tolerant probes as described herein binds to (or capable of hybridizing to) the nucleic acid component of target bacteria (such as proteobacteria). In some examples, the target bacteria (such as proteobacteria) comprises at least one sequence selected from the group consisting SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, or fragments thereof, or a sequence sharing at least about 85% sequence identity thereto. In some examples, the nucleic acid component of target bacteria (such as proteobacteria) may comprise a sequence sharing at least about 89%, or 90%, or 91%, or 92%, or 93%, or 94%, or 95%, or 96%, or 97%, or 98%, or 99% sequence identity thereto. In some examples, the nucleic acid component of proteobacteria may comprise a sequence having one, or two, or three nucleotide differences thereto.

In some examples, the method as disclosed herein detects bacterial nucleic acid molecules found in bacterium such as, but not limited to, Mycobacterium aurum, Mycobacterium branderi, Mycobacterium engbaekii, Mycobacterium senegalense, Mycobacterium shimoidei, Mycobacterium tokaiense, Mycobacterium triviale, Mycobacterium asiaticum, Mycobacterium avium, Mycobacterium celatum, Mycobacterium chubuense, Mycobacterium intracellulare, Mycobacterium kansasii, Mycobacterium szulgai, Mycobacterium terrae, Mycobacterium tuberculosis, and the like.

In some examples, the plurality of mismatch-tolerant probes as described herein binds to (or capable of hybridizing to) the nucleic acid component of target bacteria (such as a mycobacterium). In some examples, the target bacteria (such as mycobacterium) comprises at least one sequence selected from the group consisting SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 80, SEQ ID NO: 81, SEQ ID NO: 82, SEQ ID NO: 83, SEQ ID NO: 84, or fragments thereof, or a sequence sharing at least about 85% sequence identity thereto. In some examples, the nucleic acid component of target bacteria (such as mycobacterium) may comprise a sequence sharing at least about 89%, or 90%, or 91%, or 92%, or 93%, or 94%, or 95%, or 96%, or 97%, or 98%, or 99% sequence identity thereto. In some examples, the nucleic acid component of mycobacterium may comprise a sequence having one, or two, or three nucleotide differences thereto.

In some examples, the plurality of mismatch-tolerant probes is configured to have different Tm signatures for different species of bacterial nucleic acid molecules.

In some examples, the plurality of mismatch-tolerant probes has one or more of the following properties: is capable of producing a fluorescent signal; has a greater number of mismatches with the sequences of one group of bacterial nucleic acid molecules than another group of bacterial nucleic acid molecules; comprises an oligonucleotide comprising a reporter moiety and a quencher moiety; comprises a molecular beacon; each is capable of producing a distinct colorimetric signal, optionally wherein the colorimetric signal is selected from the group consisting of: a green signal, an orange signal and a red signal.

In some examples, the bacterial nucleic acid molecules in each group share at least about 97% sequence identity.

In some examples, the plurality of mismatch-tolerant probes has one or more of the following properties:
a) is capable of producing a fluorescent signal;
b) has a greater number of mismatches with the sequences of one group of bacterial nucleic acid molecules than another group of bacterial nucleic acid molecules;
c) comprises an oligonucleotide comprising a reporter moiety and a quencher moiety;
d) comprises a molecular beacon;
e) each is capable of producing a distinct colorimetric signal, optionally wherein the colorimetric signal is selected from the group consisting of: a green signal, an orange signal and a red signal.

In some examples, the plurality of mismatch-tolerant probes comprises at least one sequence selected from the group consisting of:
CCGGCCGAAGGCCTCCATCCCGCACGCGGCGTCGCTGCGTCAGGGGC CGG (SEQ ID NO: 4) or a sequence sharing at least about 85% sequence identity thereto;
CCGGCACTCACGCGGCGTTGCTGCATCAGGGTTTCCCCCATTGTGGCC GG (SEQ ID NO: 5) or a sequence sharing at least about 85% sequence identity thereto; and
CCGGCACACGCGGCATGGCTGGATCAGGCTTGCGCCCATTGTCCAGCC GG (SEQ ID NO: 6) or a sequence sharing at least about 85% sequence identity thereto. In some examples, the probes may comprise a sequence sharing at least about 89%, or 90%, or 91%, or 92%, or 93%, or 94%, or 95%, or 96%, or 97%, or 98%, or 99% sequence identity thereto. In some examples, the probes may comprise a sequence having one, or two, or three nucleotide differences thereto.

In some examples, the plurality of mismatch-tolerant probes comprises at least one sequence selected from the group consisting of:
CCGGCCGGATAGGACCACAGGATGCATGTCGTGTGGTGGAAAGCGCCG G (SEQ ID NO: 7) or a sequence sharing at least about 85% sequence identity thereto;
CCGGGCGGATAGGACCACGGGATGCATGTGTTGTGGTGGAAAGCCCCG G (SEQ ID NO: 8) or a sequence sharing at least about 85% sequence identity thereto; and
CCGGCCGAATAGGACCACGCGCTTCATGGTGTGTGGTGGAAAGCGCCG G (SEQ ID NO: 9) or a sequence sharing at least about 85% sequence identity thereto.
   In some examples, the probes may comprise a sequence sharing at least about 89%, or 90%, or 91%, or 92%, or 93%, or 94%, or 95%, or 96%, or 97%, or 98%, or 99% sequence identity thereto. In some examples, the probes may comprise a sequence having one, or two, or three nucleotide differences thereto. In some examples, the method is carried out on a digital PCR platform. In some examples, the methods as described herein may be performed in a digital PCR (dPCR) system such as microfluidic-based Fluidigm dPCR system, Combinati's Absolute Q dPCR system, and the like. In some examples, the dPCR may provide real time fluorescence tracking capability.

In another aspect, there is provided a method of determining a health of a subject, the method comprising profiling a microbiome composition in a sample from the subject according to the methods as described herein.

In another aspect, there is provided a kit for use in the methods as described herein.

In yet another aspect, there is provided a microbiotic composition profiling kit comprising a reagent for detecting a nucleic acid molecules of a microbiotic composition, and instructions to perform the methods as described herein.

In yet another aspect, there is provided a microbiotic composition profiling kit comprising primers as described herein. In some examples, the kit may further comprise mismatch-tolerant probes as described herein. In some examples, the kit may further comprise a plurality of mismatch-tolerant probes configured to have different Tm signatures for different groups of nucleic acid molecules of a microbiotic phyla/genus/species/type. In some examples, the kit may further comprise instructions to perform the methods as described herein.

In another aspect, there is provided a kit for use in the methods as described herein, the kit comprising primers comprising at least one sequence selected from the group consisting of:
ACTCCTACGGGAGGCAGCAG (SEQ ID NO: 1) or a sequence sharing at least about 85% sequence identity thereto;
ATTACCGCGGCTGCTGG (SEQ ID NO: 2) or a sequence sharing at least about 85% sequence identity thereto;
TACGGGAGGCAGCAG (SEQ ID NO: 3) or a sequence sharing at least about 85% sequence identity thereto,
CTGGCACCAAGCAGAAGACGGCA (SEQ ID NO: 66) or a sequence sharing at least about 85% sequence identity thereto;
ACGGCGACCACCGAGATCTACC (SEQ ID NO: 67) or a sequence sharing at least about 85% sequence identity thereto; and
GGCACCAAGCAGAAGA (SEQ ID NO: 68) or a sequence sharing at least about 85% sequence identity thereto.

In yet another aspect, there is provided a kit for use in the methods as described herein, the kit comprising mismatch-tolerant probes comprising at least one sequence selected from the group consisting of:
CCGGCCGAAGGCCTCCATCCCGCACGCGGCGTCGCTGCGTCAGGGGC CGG (SEQ ID NO:4) or a sequence sharing at least about 85% sequence identity thereto;
CCGGCACTCACGCGGCGTTGCTGCATCAGGGTTTCCCCCATTGTGGCC GG (SEQ ID NO: 5) or a sequence sharing at least about 85% sequence identity thereto;
CCGGCACACGCGGCATGGCTGGATCAGGCTTGCGCCCATTGTCCAGCC GG (SEQ ID NO: 6) or a sequence sharing at least about 85% sequence identity thereto;
CCGGCCGGATAGGACCACAGGATGCATGTCGTGTGGTGGAAAGCGCCG G (SEQ ID NO: 7) or a sequence sharing at least about 85% sequence identity thereto;
CCGGGCGGATAGGACCACGGGATGCATGTGTTGTGGTGGAAAGCCCCG G (SEQ ID NO: 8) or a sequence sharing at least about 85% sequence identity thereto; and/or
CCGGCCGAATAGGACCACGCGCTTCATGGTGTGTGGTGGAAAGCGCCG G (SEQ ID NO: 9) or a sequence sharing at least about 85% sequence identity thereto.

Also disclosed are kits containing reagents for performing the above-described methods, including PCR and/or probe-target hybridization reactions. To that end, one or more of the reaction components, e.g., PCR primers, polymerase, and probes, for the methods disclosed herein can be supplied in the form of a kit for use. In such a kit, an appropriate amount of one or more reaction components is provided in one or more containers or held on a substrate.

The kit also contains additional materials for practicing the above-described methods. In some examples, the kit contains some or all of the reagents, materials for performing a method that uses primers and/or probes according to the present disclosure. Some or all of the components of the kits can be provided in containers separate from the container(s) containing the primers and/or probes of the present disclosure. Examples of additional components of the kits include, but are not limited to, one or more different polymerases, one or more control reagents (e.g., probes or PCR primers or control templates), and buffers for the reactions (in 1× or concentrated forms). The kit may also include one or more of the following components: supports, terminating, modifying or digestion reagents, osmolytes, and an apparatus for detection.

The reaction components used can be provided in a variety of forms. For example, the components (e.g., enzymes, probes and/or primers) can be suspended in an aqueous solution or as a freeze-dried or lyophilized powder, pellet, or bead. In the latter case, the components, when reconstituted, form a complete mixture of components for use in an assay. The kits of the present disclosure can be provided at any suitable temperature. For example, for storage of kits containing protein components (e.g., an enzyme) in a liquid, it is preferred that they are provided and maintained below 0° C., preferably at or below −20° C., or otherwise in a frozen state.

A kit or system of this present disclosure may contain, in an amount sufficient for at least one assay, any combination of the components described herein. In some applications, one or more reaction components may be provided in pre-measured single use amounts in individual, typically disposable, tubes or equivalent containers. With such an arrangement, a PCR reaction can be performed by adding a target nucleic acid or a sample/cell containing the target nucleic acid to the individual tubes directly. The amount of a component supplied in the kit can be any appropriate amount, and may depend on the target market to which the product is directed. The container(s) in which the components are supplied can be any conventional container that is capable of holding the supplied form, for instance, microfuge tubes, ampoules, bottles, or integral testing devices, such as fluidic devices, cartridges, lateral flow, or other similar devices.

The kits can also include packaging materials for holding the container or combination of containers. Typical packaging materials for such kits and systems include solid matrices (e.g., glass, plastic, paper, foil, micro-particles and the like) that hold the reaction components or detection probes in any of a variety of configurations (e.g., in a vial, microtiter plate well, microarray, and the like). The kits may further include instructions recorded in a tangible form for use of the components.

It will be appreciated by a person skilled in the art that other variations and/or modifications may be made to the embodiments disclosed herein without departing from the spirit or scope of the disclosure as broadly described. For example, in the description herein, features of different exemplary embodiments may be mixed, combined, interchanged, incorporated, adopted, modified, included etc. or the like across different exemplary embodiments. The present embodiments are, therefore, to be considered in all respects to be illustrative and not restrictive.

### DESCRIPTION OF FIGURES

Example embodiments of the disclosure will be better understood and readily apparent to one of ordinary skill in the art from the following discussions and if applicable, in conjunction with the figures. It should be appreciated that other modifications or changes may be made without deviating from the scope of the invention. Example embodiments are not necessarily mutually exclusive as some may be combined with one or more embodiments to form new exemplary embodiments. The example embodiments should not be construed as limiting the scope of the disclosure.

FIG. 1 shows SAMBA assay workflow Schematic overview of the dPCR (SAMBA) assay. The DNA sample is first diluted and separated into single copies in a dPCR chip. Asymmetric PCR is performed to generate single stranded DNA targets of the 16S rRNA gene. Three SMBs will bind to these single stranded DNA targets with different mismatches as indicated by the pink bars. For each bacterial identity, a unique Tm signature will be formed with the three SMBs. As dPCR allows absolute counting of the chambers with the same Tm signatures, the proportion of each bacterial identity within the sample can be quantified.

FIG. 2 shows SAMBA enables highly multiplexed quantitative detection of mixed bacteria species. (A) Characteristic melt curve profiles in multiple fluorescence channels enable robust bacteria species identification from single DNA template molecule. (B) Tm signatures from 3 fluorescence channels of 16 bacteria species obtained using SAMBA. (C) Representation of Tm signatures with SAMBA Scores shows 16 distinct clusters that consists of bacteria from the same species. (D) Linear decision boundaries in SAMBA 1 Score enables accurate classification of 4 bacteria species M. aurum, M. branderi, M. shimoidei and M. triviale. Samples containing mixed bacteria species of different ratios in the order above (Equal—25%:25%:25%:25%, Staggered—53:3%:26.7%:13.3%:6.1%) are accurately quantified using SAMBA.

FIG. 3 shows probe design guided by phylum-level differences in 16S rRNA gene. (A) Illustration showing the positions in the 16S V3 region targeted by each probe. (B) Sequence logos for each probe target region. The nucleotides in red are positions where there are high degree of mismatch with the probe sequence. (C) The percent of mismatches of each probe against sequences within each phylum HEX, FAM and ROX probes contain least mismatch against Actinobacteria, Firmicutes and Proteobacteria respectively. (D) 3D bubble plot showing distinct clustering of gut microbiome sequences based on predicted Tm signatures, providing theoretical validation the probes used in the SAMBA assay.

FIG. 4 shows validation of SAMBA Assay for microbiome profiling using synthetic oligonucleotide pools. (A) Tm profiles of synthetic oligonucleotide pools obtained by SAMBA assay shows a distinct signature according to phylum. (B) The SAMBA Score representation of the combined Tm information from multiple fluorescence channel allows accurate phylum classification with linear decision boundaries. Samples containing mixed oligonucleotide pools from three phyla (Firmicutes, Actinobacteria, Proteobacteria) at different ratios (Uniform—33%:33%:33%, Staggered—67%:27%:5%) can be quantified using SAMBA with good accuracy. Each bubble represents a SAMBA OTU and its size is proportional to abundance.

FIG. 5 shows microbiome profiling of clinical samples with SAMBA shows phylum and SAMBA OTU level differences between CRC patients and healthy subjects. (A) NMDS plot based on Bray-Curtis dissimilarity among the SAMBA OTUs reveal distinction between microbial communities in healthy subjects and CRC patients. (B) Microbiome phylum compositions measured using SAMBA in clinical samples recapitulates key trends in NGS analysis. (C) High concordance between quantitative microbiome analyses performed using SAMBA and NGS. (D) At the phylum level, Firmicutes abundance is significantly higher and Actinobacteria abundance is significantly lower in CRC patients (n=4) compared to healthy subjects (n=4) measured by SAMBA assay. (E) SAMBA can enable differential analysis of microbiome composition at higher taxonomic resolution. Two SAMBA OTUs are significantly enriched in CRC patients compared to heathy subjects.

FIG. 6 shows melt curve profiles of remaining 12 bacteria species in SAMBA.

FIG. 7 shows (A) Naïve Bayes model for classifying 16 bacteria species using three SMB probes, obtained from training with 1680 (70%) SAMBA measurements; (B) prediction results of Naïve Bayes classifier on 720 (30%) SAMBA measurements used for validation. High accuracy of 98.3% is demonstrated.

FIG. 8 shows (A) decision tree model for classifying 16 bacteria species using SAMBA 1 Score, obtained from training with 420 (70%) SAMBA measurements; (B) prediction results of decision tree classifier on 180 (30%) SAMBA measurements used for validation. High accuracy of 98.3% is demonstrated.

FIG. 9 shows the percent of mismatches of three SMB probes against sequences within the phyla Actinobacteria, Firmicutes, Proteobacteria and Bacteriodetes. Bacteriodetes sequences have high degree of mismatches with all three probes, and they would not be detected via SAMBA due to the specific probe design.

FIG. 10 shows principal component analysis of the melting temperatures of DNA oligonucleotide pools from SAMBA assay. Measurements are aggregated into SAMBA OTUs and size of bubbles is proportional to the abundance of specific SAMBA OTUs. PC 2 is observed to provide good separation among the three phyla and is chosen to represent the SAMBA Score for microbiome profiling.

FIG. 11 shows (A) decision tree model for classifying 3 phyla using SAMBA Score, obtained from training with 340 (70%) SAMBA measurements of DNA oligonucleotide pools; (B) prediction results of decision tree classifier on 146 (30%) SAMBA measurements used for validation. High accuracy of 95.2% is demonstrated.

### EXPERIMENTAL SECTION

The present disclosure presents a method called Split, Amplify and Melt analysis of BActeria-community (SAMBA), which combines probe-based dPCR technology with multicolor melting analysis to significantly expand its multiplexing capability for analysis of mixed bacteria communities. The inventors of the present disclosure developed a set of multicolor molecular beacon probes that have differential binding affinity to 16S rDNA sequences from bacteria of different taxonomic groups. Simultaneous melting analysis of these probe hybridization with amplification product of single 16S rDNA molecule in individual partition within a dPCR platform yield unique melting temperature (Tm) signatures reflective of the species taxonomy. This analysis provides information about the absolute abundance of various taxonomies of the microbiome sample of interest at the phylum level. The present study validated SAMBA in silico, in vitro, and also on clinical samples. The inventors of the present disclosure demonstrated the ability of SAMBA to distinguish 16 different bacteria species—a multiplexing capability far greater than any dPCR method reported to date. The inventors of the present disclosure showed that SAMBA is capable of measuring subtle but consistent changes in gut microbiome composition between healthy and colorectal cancer patients, and the results are well-correlated with concurrent next generation sequencing analysis. Leveraging on existing capabilities of array-based dPCR platforms, SAMBA is a promising method for rapid, user-friendly, and high-throughput profiling of microbiome in clinical laboratories.

### Materials and Methods

qPCR Assay:

To determine the Tm signatures of the 16 mycobacteria species with qPCR, 1× TaqMan Fast Advanced Master mix (Applied Biosystems), 50 nM Forward primer, 50 nM Reverse primer, 1 μM Excess primer, 50 nM FAM probe, 100 nM HEX probe, 100 nM ROX probe (primer and probe sequences in Table 2), 1 μL ddH₂O and 1 μL 0.1 μM of synthetic bacteria sequences (Table 1) were mixed in a 96-well PCR plate. Asymmetric PCR and melting curve analysis were performed on a qPCR platform (Biorad CFX96 Touch Real-Time PCR Detection System). The PCR thermal conditions were 1 cycle of 95° C. for 2 min, 20 cycles of 95° C. for 10 s, 61° C. for 30 s and 30 cycles of 95° C. for 10 s, 50° C. for 15 s, and 60° C. for 15 s. To form hybrids, the reaction mixture was incubated at 95° C. for 5 min before cooling gradually to 45° C. and holding at 45° C. for 5 min. The reaction mixture was heated gradually from 45° C. to 85° C. at 1° C. interval, holding each temperature for 15 s, with continuous monitoring of the fluorescence during the process for melting curve analysis.

TABLE 1
DNA sequence of the 16 mycobacteria species tested

| Species | Sequence | SEQ ID NO: |
|---|---|---|
| M. aurum | TGCTGGCACCAAGCAGAAGACGGCATAG | 69 |
| | CTTTCCACCACACGACATGCATCGCGTAG | |
| | TCCTATTCGGTAGATCTCGGTGGTCGCC | |
| | GTATC | |
| M. branderi | TGCTGGCACCAAGCAGAAGACGGCATAG | 70 |
| | CTTTCCACCACACACCATGCAGCATGTGG | |
| | TCCTATCCGGTAGATCTCGGTGGTCGCC | |
| | GTATC | |
| M. engbaekii | TGCTGGCACCAAGCAGAAGACGGCATAG | 71 |
| | CTTTCCACCACACACCATGAAGCGCGCG | |
| | GTCCTATCCGGTAGATCTCGGTGGTCGC | |
| | CGTATC | |
| M. senegalense | TGCTGGCACCAAGCAGAAGACGGCATAG | 72 |
| | CTTTCCACCACACACCATGAAGCGCGTG | |
| | GTCCTATCCGGTAGATCTCGGTGGTCGC | |
| | CGTATC | |
| M. shimoidei | TGCTGGCACCAAGCAGAAGACGGCATAG | 73 |
| | CTTTCCACCACACACCATGCGACATGTGG | |
| | TCCTATCCGGTAGATCTCGGTGGTCGCC | |
| | GTATC | |
| M. tokaiense | TGCTGGCACCAAGCAGAAGACGGCATAG | 74 |
| | CTTTCCACCACAGCACATGAATGCCGTGG | |
| | TCCTATTCGGTAGATCTCGGTGGTCGCC | |
| | GTATC | |
| M. triviale | TGCTGGCACCAAGCAGAAGACGGCATAG | 75 |
| | CTTTCCACCACACACCATTCGATGCGCGG | |
| | TCCTATCCGGTAGATCTCGGTGGTCGCC | |
| | GTATC | |
| M. asiaticum | TGCTGGCACCAAGCAGAAGACGGCATAG | 76 |
| | CTTTCCACCACAGGACATGCATCCCGTG | |
| | GTCCTATCCGGTAGATCTCGGTGGTCGC | |
| | CGTATC | |
| M. avium | TGCTGGCACCAAGCAGAAGACGGCATAG | 77 |
| | CTTTCCACCAGAAGACATGCGTCTTGAGG | |
| | TCCTATCCGGTAGATCTCGGTGGTCGCC | |
| | GTATC | |
| M. celatum | TGCTGGCACCAAGCAGAAGACGGCATAG | 78 |
| | CTTTCCACCACAAGACATGCATCCCATGG | |
| | TCCTATCCGGTAGATCTCGGTGGTCGCC | |
| | GTATC | |
| M. chubuense | TGCTGGCACCAAGCAGAAGACGGCATAG | 79 |
| | CTTTCCACCACAGCACATGCATGCCGTG | |
| | GTCCTATCCGGTAGATCTCGGTGGTCGC | |
| | CGTATC | |
| M. intracellulare | TGCTGGCACCAAGCAGAAGACGGCATAGCTT | 80 |
| | TCCACCTAAAGACATGCGCCTAAAGGTCCTAT | |
| | CCGGTAGATCTCGGTGGTCGCCGTATC | |
| M. kansasii | TGCTGGCACCAAGCAGAAGACGGCATAG | 81 |
| | CTTTCCACCACAAGGCATGCGCCAAGTG | |
| | GTCCTATCCGGTAGATCTCGGTGGTCGC | |
| | CGTATC | |
| M. szulgai | TGCTGGCACCAAGCAGAAGACGGCATAG | 82 |
| | CTTTCCACCCCAAGGCATGCGCCTCGGG | |
| | GTCCTATCCGGTAGATCTCGGTGGTCGC | |
| | CGTATC | |
| M. terrae | TGCTGGCACCAAGCAGAAGACGGCATAG | 83 |
| | CTTTCCACCACAGAACATGCATCCCATGG | |
| | TCCTATCCGGTAGATCTCGGTGGTCGCC | |
| | GTATC | |
| M. tuberculosis | TGCTGGCACCAAGCAGAAGACGGCATAG | 84 |
| | CTTTCCACCACAAGACATGCATCCCGTGG | |
| | TCCTATCCGGTAGATCTCGGTGGTCGCC | |
| | GTATC | |

TABLE 2
Probe sequences and primers used for SAMBA assay on Mycobacterial
species.

| Probe | DNA sequence |
|---|---|
| HEX Probe | /5HEX/CCGGCCGGATAGGACCACAGGATGCATGTCGTGTGGTG |
| | GAAAGCGCCGG/3BHQ_1/ (SEQ ID NO: 7) |
| FAM Probe | /56_FAM/CCGGGCGGATAGGACCACGGGATGCATGTGTTGTGG |
| | TGGAAAGCCCCGG/3BHQ_1/ (SEQ ID NO: 8) |
| ROX Probe | /56_ROXN/CCGGCCGAATAGGACCACGCGCTTCATGGTGTGTG |
| | GTGGAAAGCGCCGG/3BHQ_2/ (SEQ ID NO: 9) |
| Forward primer | CTGGCACCAAGCAGAAGACGGCA (SEQ ID NO: 66) |
| Reverse primer | ACGGCGACCACCGAGATCTACC (SEQ ID NO: 67) |
| Excess primer | GGCACCAAGCAGAAGA (SEQ ID NO: 68) |

### SAMBA Assay:

The inventors of the present disclosure utilized the Fluidigm 48.770 digital array integrated fluidic circuit (IFC) (100-6151) that contains 48×770 compartments (0.85 nl each compartment). Template DNA was diluted to 10 ag μl⁻¹ (final concentration) to efficiently partition single 16S rDNA in individual compartments in the dPCR chip.

To perform SAMBA assay on the 16 mycobacteria DNA, the mastermix used for the dPCR contained 50 nM FAM Probe, 100 nM HEX Probe, 100 nM ROX Probe, 50 nM Forward primer, 50 nM Reverse primer, 1 μM Excess primer, 2 mM MgCl₂, 1× GE Sample Loading Reagent and 1× TaqMan Fast Advanced Master mix. The probe and primer sequences can be found in Table 2.

To perform SAMBA assay on the three synthetic oligonucleotide pools (Actinobacteria, Firmicutes, Proteobacteria) and clinical samples, contained 50 nM FAM Probe, 100 nM HEX Probe, 100 nM ROX Probe, 50 nM 338 Forward primer, 50 nM 534 Reverse primer, 1 μM V3 excess primer, 2 mM MgCl₂, 1× GE Sample Loading Reagent and 1× TaqMan Fast Advanced Master mix. The probe and primer sequences can be found in Table 4.

After loading all the samples into the dPCR chip, dPCR was performed according to the manufacturer's instructions in the Fluidigm Biomark HD system using the following thermal conditions. The PCR thermal conditions for the 16 mycobacteria samples are 1 cycle of 95° C. for 2 min, 20 cycles of 95° C. for 10 s, 61° C. for 30 s and 30 cycles of 95° C. for 10 s, 50° C. for 15 s, 60° C. for 15 s with fluorescence monitoring for the first 20 cycles of PCR. The PCR thermal conditions for synthetic oligonucleotide pools and clinical samples are 1 cycle of 95° C. for 2 min, 20 cycles of 95° C. for 10 s, 62° C. for 15 s, 72° C. for 15 s and 30 cycles of 95° C. for 10 s, 52° C. for 15 s, 72° C. for 15 s with fluorescence monitoring for the first 20 cycles of PCR. To form hybrids, the reaction mixture was incubated at 95° C. for 5 min before cooling gradually to 45° C. and holding at 45° C. for 5 min. The reaction mixture was heated gradually from 45° C. to 85° C. at 1° C. interval with continuous monitoring of the fluorescence during the process. Exposure times for all the SMB probes were inputted manually (Passive reference ROX=4 s, FAM=12 s, HEX=5 s, ROX=3 s). The option of “Capture first image only” was selected for the passive reference dye. The Fluidigm Digital PCR Analysis Program (version 4.1.2) was used to extract Tm values for all three probes from each chamber after the dPCR run.

TABLE 4
Probe and primer sequences used for SAMBA assay on human microbiome
samples

| Probe/Primer | DNA sequence |
|---|---|
| HEX Probe | /5HEX/CCGGCCGAAGGCCTCCATCCCGCACGCGGCGTCGCTGC |
| | GTCAGGGGCCGG/3BHQ_1/ (SEQ ID NO: 4) |
| FAM Probe | /56_FAM/CCGGCACTCACGCGGCGTTGCTGCATCAGGGTTTCCC |
| | CCATTGTGGCCGG/3BHQ_1/ (SEQ ID NO: 5) |
| ROX Probe | /56_ROXN/CCGGCACACGCGGCATGGCTGGATCAGGCTTGCGCC |
| | CATTGTCCAGCCGG/3BHQ_2/ (SEQ ID NO: 6) |
| 338 Forward primer | ACTCCTACGGGAGGCAGCAG (SEQ ID NO: 1) |
| 534 Reverse primer | ATTACCGCGGCTGCTGG (SEQ ID NO: 2) |
| V3 excess primer | TACGGGAGGCAGCAG (SEQ ID NO: 3) |

### Data Analysis for 16 Mycobacteria Species:

Tm values in each compartment were extracted by the Fluidigm Digital PCR Analysis Program. Compartments that contain in-range Tm's (50° C. to 85° C.) in at least two separate fluorescence channels were considered as positive compartments. An undetectable Tm in any fluorescence channel of a positive compartment was defaulted to 40° C. Naïve Bayes model for classifying 16 mycobacteria species from Tm's of 3 fluorescence channel is trained with 70% of the measurements (n=1680) using the free and open-source KNIME software (https://www.knime.com/) and validated with the remaining 30% of the measurements (n=720).

SAMBA 1 Score and SAMBA 2 Score were calculated for each positive compartments according to the formula in the main text. Decision Tree model for classifying 4 mycobacteria species from SAMBA 1 Score was trained with 70% of the measurements (n=420) using KNIME and validated with the remaining 30% of the measurements (n=180). Based on the decision tree model, the identities of positive compartments were identified based on SAMBA 1 Scores. This criterion was applied for measuring the abundance of each bacteria in the subsequent mixed species experiment.

### Data Analysis for Synthetic Oligonucleotide Pools and Clinical Samples:

Tm values in each compartment was extracted by the Fluidigm Digital PCR Analysis Program. Compartments that contain in-range Tm's (50° C. to 85° C.) in at least one fluorescence channel were considered as positive compartments. An undetectable Tm in any fluorescence channel of a positive compartment was defaulted to 40° C. To enable abundance analysis of highly complex mixtures, the Operational Taxonomic Unit (OTU) concepts in microbial ecology studies was adopted. The Tm's in positive compartments were rounded to the nearest integer. SAMBA measurements that had the same triplet of rounded Tm's were considered to be in the same SAMBA OTU. SAMBA OTUs that had only a single occurrence in the present data were removed to reduce noise in accordance to common practice in microbial ecology to remove singleton OTUs.

SAMBA measurement results from the three oligonucleotide pools were combined and their Tm profiles were analyzed using Principal Component Analysis to perform dimensionality reduction and find linear combinations of Tm's that can best separate the different phyla. PC 2 was eventually selected as the SAMBA Score as it could effectively be used to identify the phylum of a template DNA. Decision Tree model for classifying the phylum from SAMBA Score was trained with 75% of the measurements (n=340) using KNIME and validated with the remaining 25% of the measurements (n=146). This decision tree model was applied for measuring the abundance of each phylum in the subsequent mixed synthetic oligonucleotide pool experiment and clinical microbiome profiling.

NMDS analysis of the SAMBA OTUs from clinical samples was performed using the Phyloseq package in R with Bray-Curtis dissimilarity as a distance function. The phyla and SAMBA OTUs that were significantly different between CRC patients and healthy subjects were identified by comparing the microbiome compositions measured from SAMBA assays from the two groups and performing two-tailed t-tests.

Clinical Sample Collection, Extraction, and 16S rRNA Gene Amplicon Sequencing

All stool samples were collected immediately after defecation in a sterile stool collection tube containing 4 sterile glass beads measuring 5 mm in diameter (Merck) and 2 ml of RNAlater® (Invitrogen). Samples were immediately and transported to tissue repository at NUH and stored at −80° C.

Genomic DNA was extracted from human stool samples using DNeasy Powersoil Pro Kit (Qiagen) according to the manufacturer's instructions with additional steps at the start. The stool samples were thawed at room temperature before being vortexed to resuspend the material. Sufficient volume of stool slurry was taken to make up 0.25 g per sample before transferring it to a 1.5 ml tube. Sample was centrifuged at 14,000 g for 5 min before decanting and resuspending in 1 ml of PBS. This process was performed thrice with the resuspension in the last repeat using 800 μL of Solution CD1 instead of 1 ml of PBS. Subsequent steps were the same as manufacturer's instructions. Each DNA sample was quantified using a Qubit Fluorometer (Thermo Fisher) for concentration.

A PCR targeting the V3-V4 region using forward (5′-CCTACGGGNGGCWGCAG) and reverse primers (5′-GACTACHVGGGTATCTAATCC) with overhang adaptors, were performed as recommended by the 16S metagenomics library kit by Illumina. The quality and quantity of the amplicons were measured using Agilent 4200 TapeStation, picogreen and nanodrop. All the samples passed the quality control measurement and proceed straight for a second round of PCR step for library preparation. The quality of libraries was measured using Agilent 4200 TapeStation, picogreen and qPCR. Libraries that pass the quality control measurement were pooled according to the protocol recommended by Illumina and was sequenced using the MiSeq platform using the 2×301 PE format.

### Sequencing Data Analysis

Sequences obtained from Miseq platform (Illumina, San Diego, CA, United States) were processed using Mothur (version 1.44.1). First, the forward and reverse reads were combined, then sequences with ambiguous bases or lengths longer than expected were removed. Duplicate sequences were also merged. Sequences resulting from sequencing errors were removed. Next, sequences were pre-clustered before removing chimeras using VSEARCH algorithm. To prepare the sequences for analysis, they were grouped into Operational Taxonomic Units (OTUs). Sequences in the same OTUs are at least 97% similar. Subsequently, the taxonomy of each OTU was identified using the SILVA bacteria database.

### Clinical Samples

The study was approved by the National Healthcare Group Domain Specific Review Board (NHG DSRB Ref: 2017/01257) for use of fecal samples obtained from recruited subjects from National University Hospital, Singapore. All subjects were recruited with informed consent of age 21 and above and were scheduled to undergo elective diagnostic colonoscopy. Subjects who were found to have colorectal cancer at colonoscopy were assigned as “cancer”, while those who did not have cancer were assigned as “healthy”. 63 fecal samples were used for this study and eight samples (C1 to C4 and H1 to H4) were analysed by SAMBA (Table 6). All fecal samples collected in this study were stored at −80° C. prior to downstream processes.

TABLE 6
Demographic and clinical data of CRC (C1 to C4) and healthy subjects
(H1 to H4).

| | | | | Colonic tumor | TNM Cancer |
|---|---|---|---|---|---|
| Subject | Age | Gender | Race | location | stage |
| C1 | 56 | Female | Malay | Sigmoid | 1 |
| C2 | 56 | Female | Malay | Sigmoid | 1 |
| C3 | 46 | Male | Chinese | Rectum | 3 |
| C4 | 59 | Female | Indian | Rectum | 2 |
| H1 | 57 | Female | Chinese | — | — |
| H2 | 75 | Female | Chinese | — | — |
| H3 | 61 | Male | Chinese | — | — |
| H4 | 66 | Male | Chinese | — | — |

### Results

### The Principle of SAMBA Assay

The present disclosure proposes that melting curve analysis in dPCR is a promising strategy for highly multiplexed target detection. Sloppy molecular beacons (SMB) are a class of mismatched-tolerant probes that can generate specific Tm values for DNA sequences that differed by as little as one nucleotide. Combining the Tm values generated by several probe-target hybrids can result in Tm signatures that serve as highly accurate sequence identifier. Previously SMBs have been used to identify 27 different mycobacteria species, and 119 different blood-based pathogens in cultures, demonstrating their extensive multiplexing capability. However, these previously reported platforms utilizing SMB probes are not suitable for profiling mixtures of a community of bacteria due to the inevitable merging of melting curves from different species in a complex mixture.

In a departure from previous approaches, the strategy as described herein performs SMB melting curve analysis in a dPCR platform where individual bacteria DNA is confined in separate partitions. Tm signatures from many individual bacteria can be obtained in a single dPCR chip, providing information about the identity and abundance of all the bacteria. The approach is referred to as Split, Amplify and Melt analysis of BActeria-community (SAMBA) to highlight the exploitation of melting curve analysis for highly multiplexed digital bacteria nucleic acid testing.

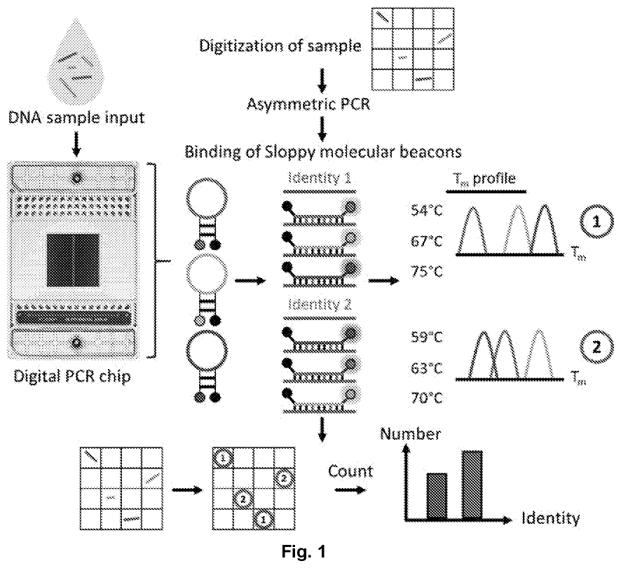

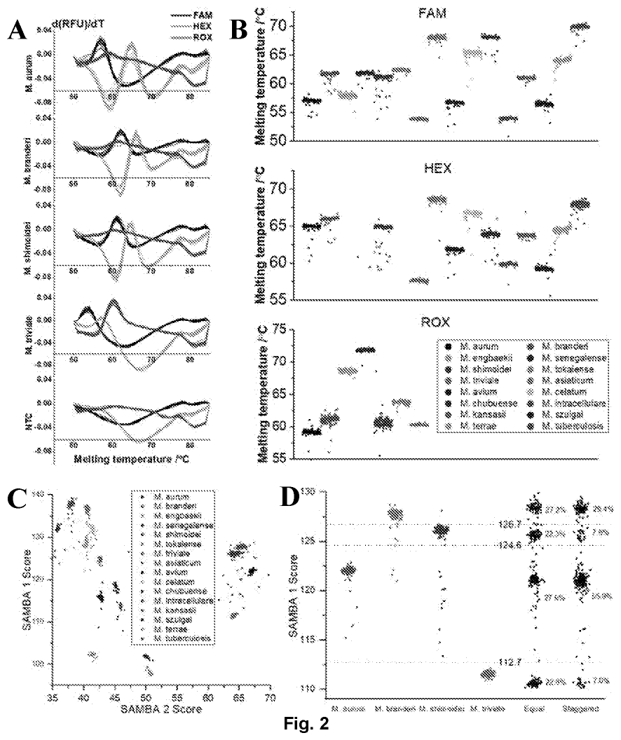

A schematic illustration of the SAMBA assay is shown in FIG. 1. The first step of SAMBA assay is to encapsulate single 16S rDNA molecules into individual compartments in a dPCR platform. The SAMBA assay leveraged on the microfluidic-based Fluidigm dPCR system, as it has a high number of partitions (˜37,000) and is capable of performing real-time melting curve analysis. Sample containing 16S rDNA is diluted, mixed with specific primers, SMB probes, and PCR mix, and partitioned into individually isolated nanoliter compartments in the dPCR chip. With limited dilution, most of the compartments will contain either one or zero copies of 16S rDNA. Subsequently, an asymmetric PCR step is performed within each of the nanoliter compartments in parallel to generate many identical copies of single stranded DNA capable of hybridizing to the SMB probes included in the mix. This amplification step is key to obtaining a robust and sensitive melting curve analysis starting from a single copy of DNA in each compartment.

The binding affinity of SMB probes to target DNA is measured by melting curve analysis. At low temperatures, a large fraction of the SMB probes can bind to its target, even when there are significant numbers of mismatches, hence a high fluorescence signal is observed. As the temperature increases, weakly-hybridizing probe-targets would start to dissociate, leading to lowered fluorescence signals in compartments containing targets that have large number of mismatches with probes. Compartments containing strongly hybridizing probe-targets with minimal number of mismatches retain fluorescence signals at high temperatures. The simultaneous use of different color probes that form different-affinity hybrids with the target allows combinatorial melting profiles to be obtained and significantly increase the multiplexing capacity. The hybridization behaviour of SMB probes to targets is represented by the Tm, which is the temperature corresponding to the maximum of the negative derivative of normalized fluorescence in each compartment. As each target has a unique pattern of mismatches to the tested panels of SMB probes, the combined Tm of all SMB probes would provide a fingerprint for unique identification of that target. The enumeration of compartments that have the same Tm fingerprint provides absolute quantification of the abundance of a particular species.

### Optimization and Validation of the SAMBA Assay.

To perform a proof of concept of the SAMBA assay for measuring absolute composition of a complex mixture, synthetic DNA sequences corresponding to different species of mycobacteria was used. It was previously shown that a panel of 4-colored SMB probes can be used to distinguish between 27 pure mycobacterial species (El-Hajj, H. H. et al., 2009). The present inventors synthesized three SMB probes (Table 6) to test the ability to distinguish between 4 mycobacterial 16S rDNA sequences.

TABLE 6
Probe sequences used for SAMBA assay
on Mycobacterial species.

| Probe | DNA sequence |
|---|---|
| Probe A | /5HEX/CCGGCCGGATAGGACCACAGGATGCATGTCGTGT |
| (HEX) | GGTGGAAAGCGCCGG/3BHQ_1/ (SEQ ID NO: 7) |
| Probe B | /56-FAM/CCGGGCGGATAGGACCACGGGATGCATGTGTT |
| (FAM) | GTGGTGGAAAGCCCCGG/3BHQ_1/ (SEQ ID NO: 8) |
| Probe C | /56-ROXN/CCGGCCGAATAGGACCACGCGCTTCATGGTG |
| (ROX) | TGTGGTGGAAAGCGCCGG/3BHQ_2/ (SEQ ID NO: |
| | 9) |

The original Linear-After-The-Exponential (LATE)-PCR protocol (Sanchez, J. A., Pierce, K. E., Rice, J. E. & Wangh, L. J. 2004) was tested for generating single-stranded DNA products for SMB probe binding. However, this protocol did not produce sufficient single-stranded DNA product for robust Tm measurement, especially when single-copy target sequence is used. The present inventors successfully implemented an asymmetric PCR approach that uses a pair of long outer primers with high annealing temperature and a nested short primer at high concentration with lower annealing temperature and performed PCR with high annealing temperature followed by low annealing temperature to obtain high single-stranded DNA product yield for SAMBA (Tang, X., Morris, S. L, Langone, J. J. & Bockstahler, L. E. 2006).

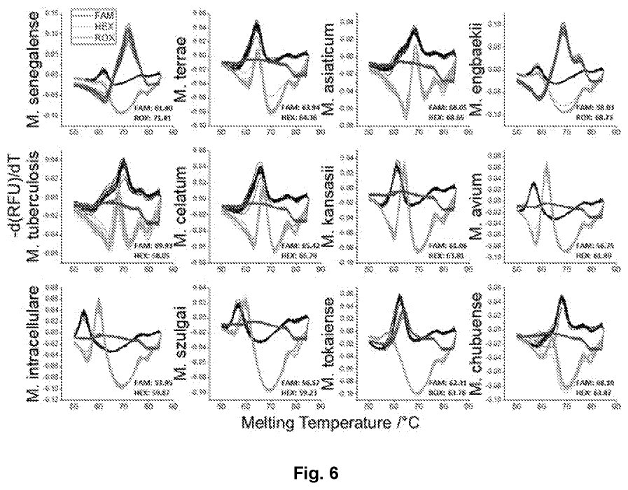

Asymmetric PCR followed by melting curve analysis of SMB probe hybridization on each of the 16 mycobacteria sequences on a conventional qPCR platform allowed the present experiment to determine the unique Tm signature of each species (Table 3). SAMBA was performed on limited dilutions of each of pure species in the dPCR platform and observed digital amplification with the number of positive compartments corresponding to input amounts, a hallmark of dPCR. FIG. 2A shows the raw melting curves in the first 20 positive compartments in digital melting PCR for four different mycobacteria species, and 20 melting curves from a no template control. The raw melting curves in dPCR of the remaining 12 mycobacteria species are included in FIG. 6). Among positive compartments containing the same mycobacteria species DNA, the melting curve profiles in all three fluorescence channels are highly consistent and reproducible, and a single peak in each fluorescence channel, allows unambiguous determination of Tm. Meanwhile different mycobacteria species DNA produce melting curve profiles that differ significantly in one or more fluorescence channel. FIG. 2A further shows that different species can be separated even when their melting temperatures in a particular fluorescence channel are indistinguishable. For example, M. aurum and M. shimoidei have overlapping Tm in the HEX channel, but the well-separated Tm values in the FAM and ROX channel allows us to easily identify the species. This highlights the significantly enhanced specificity and multiplexing capacity provided by multicolor probes compared to single-channel melting curve analysis such as HRM. These results showed that asymmetric PCR from single molecules in nanoliter compartment is robust, and the melting curve analysis of SMB probes binding to its cognate target is highly specific on a widely accessible commercial dPCR platform.

The Tm values in three fluorescence channels for all 16 mycobacteria in dPCR are shown in FIG. 2B (Tm's from 150 positive chambers are shown for each species). The results as currently presented showed that the characteristic Tm signatures of each mycobacteria is highly specific and reproducible even when starting from single template molecule in nanoliter compartment. The Tm values obtained for individual mycobacteria sequence in a dPCR platform are similar to the Tm values obtained on qPCR platform (Table 3), showing that melting profile is characteristic of the specific target and probes, regardless of the platforms used.

TABLE 3
Melting temperatures of the three sloppy molecular probes on 16
different mycobacterial species measured on a qPCR platform

| Mycobacteria species | FAM | HEX | ROX |
|---|---|---|---|
| M. senegalense | 61 | 60 | 71 |
| M. asiaticum | 67 | 67 | — |
| M. aurum | 57 | 64 | 58 |
| M. avium | 56 | 61 | — |
| M. branderi | 62 | 66 | 61 |
| M. celatum | 65 | 65 | — |
| M. chubuense | 67 | 64 | — |
| M. engbaekii | 57 | 58 | 68 |
| M. intracellulare | 53 | 59 | — |
| M. kansasii | 60 | 63 | — |
| M. shimoidei | 61 | 64 | 60 |
| M. szulgai | 56 | 58 | — |
| M. terrae | 63 | 63 | — |
| M. tokaiense | 61 | 57 | 63 |
| M. triviale | 54 | 58 | 60 |
| M. tuberculosis | 69 | 66 | — |

As the superior multiplexing capability of SAMBA lies in its ability to combine Tm information from multiple fluorescence channels, the inventors of the present disclosure further explored strategies to express the multicolor melting profiles of individual compartments as unique “scores” that will allow easy determination and visualization of the bacteria identity in each nanoliter compartment. The present study introduces the concept of SAMBA Scores, which are linear combinations of Tm measurements that consider the relationships among Tm's in individual compartments. For this case, the present study defines

$$SAMBA Score 1 = T {m}_{F A M} + T {m}_{H E X} ,$$

$$SAMBA Score 2 = - T {m}_{F A M} + {Tm}_{H E X} + T {m}_{R O X} .$$

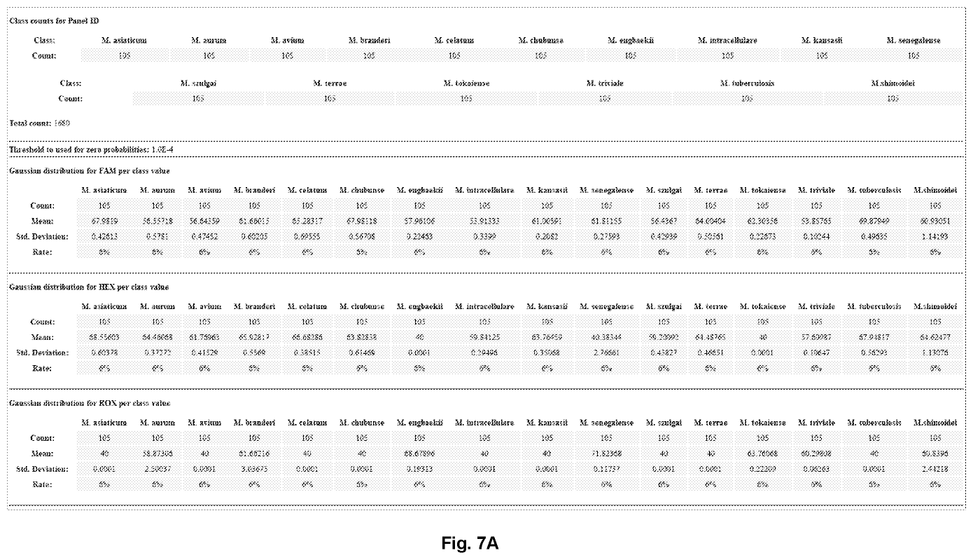

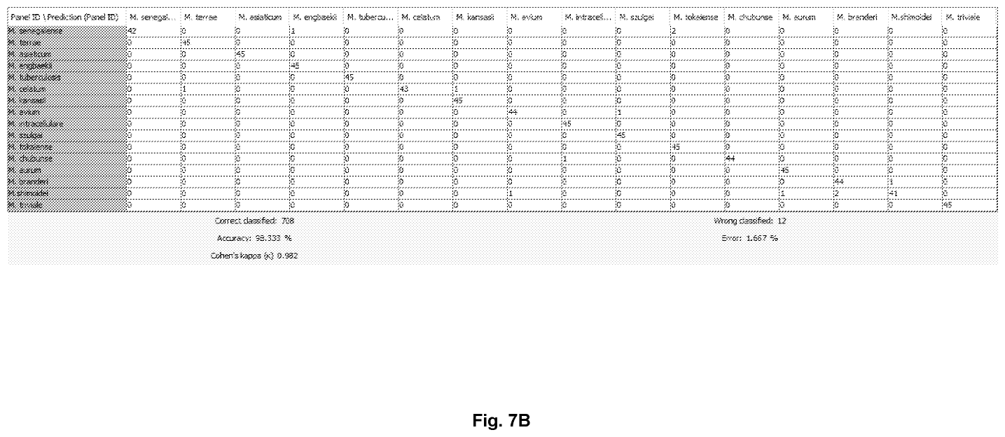

SAMBA Score 1 and SAMBA Score 2 are orthogonal vectors to each other and are chosen for each set of probes to produce good separation of different bacteria species. In some cases when a positive chamber does have an undetectable Tm at any fluorescence channel, presumably due to weak affinity of SMB probes, the undetectable Tm was defaulted to be 40° C. for the purpose of analysis. FIG. 2C shows the SAMBA 1 and SAMBA 2 scores of the 16 mycobacteria species in dPCR. Positive compartments loaded with individual templates from specific bacteria yield SAMBA scores that cluster tightly with those of the same species. Meanwhile, the SAMBA scores from compartments of different species are well separated. To assess the classification accuracy of individual bacteria DNA template by SAMBA Scores, a Naïve Bayes classifier (FIG. 7A) was trained using 70% (n=1680) of the SAMBA scores from 16 different species in dPCR and validated the accuracy of this classifier using the remaining 30% (n=720) of the SAMBA scores. This classifier is highly accurate (98.3%, FIG. 7B), further supporting the establishment of SAMBA as a highly accurate digital PCR platform with highest multiplexing capability to date (16-plex demonstrated) for single molecule profiling.

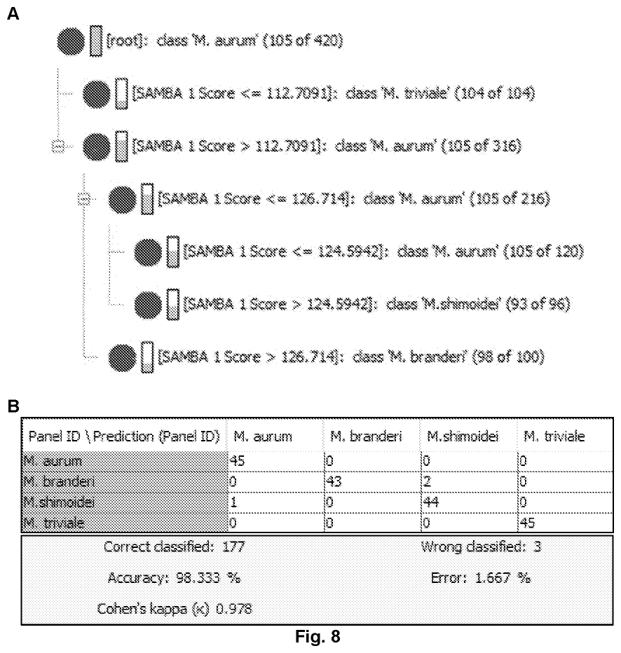

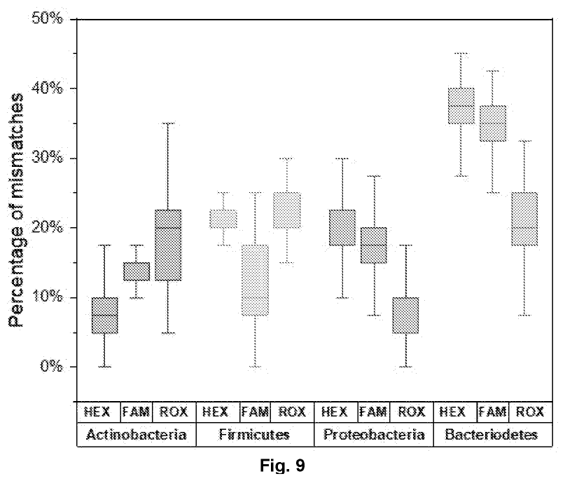

The use of dPCR allows absolute quantification of DNA molecules. In the context of SAMBA, it allows precise quantification of the composition of many mixed species—a key requirement for microbiome analysis. To validate this, mock microbial communities that mimic microbiome samples. The inventors observed that although both SAMBA 1 and SAMBA 2 Scores are needed to distinguish 16 bacteria species, SAMBA 1 Score alone is sufficient to separate four mycobacteria (M. aurum, M. branderi, M. shimoidei and M. triviale) (FIG. 2D). As a proof of concept of absolute quantification with SAMBA, a simple decision tree classifier in a single dimension will allow straightforward interpretation and visualization of a mixture of these four mycobacteria, though the inventors note that other classifiers such as Naïve Bayes would be more appropriate for quantification of other bacteria that are not well separated in one dimension. A simple decision tree classifier was trained using 70% of the SAMBA 1 Score from these four species in dPCR (FIG. 8A). A high accuracy of 98% was obtained when this classifier was validated with the SAMBA 1 Scores of the remaining 30% measurements (FIG. 8B). SAMBA was performed on the first mock sample containing even mixtures of the four bacteria. Compartments in the same array that contains four different fluorescence melting points were identified (data not shown). The mean fluorescence intensity and the negative derivative of fluorescence were plotted with respect to fluorescence to illustrate the distinct melting curve profiles in these compartments that allow the present study to infer the identities and abundance of the different bacteria species using SAMBA. SAMBA analysis of this sample showed a uniform distribution of each of the four mycobacteria species (FIG. 2D), as expected from the initial mixture. A second sample containing a staggered mixture with different proportions of four mycobacterial species (53.3%, 26.7%, 13.3%, 6.7%) was prepared. The abundance of each species in this mixture measured by SAMBA assay (55.9%, 29.4%, 7.6%, 7.0%) is close to the theoretical expected value (FIG. 2D) and provides a basis for using this platform to perform quantitative analysis of mixed bacteria in a rapid manner.

### Development of SAMBA Assay for Gut Microbiome Characterization

Microbes are an integral part of the human gut with approximately 100 trillion cells residing in the intestinal tract and are key contributors to human metabolism. Studies have shown that changes to the composition of the gut microbiota have been linked to many diseases and conditions such as Clostridium difficile infection, inflammatory bowel disease, colorectal cancer (CRC) and, human immunodeficiency virus infection.

The four predominant phyla in the gut are Firmicutes, Bacteriodetes, Actinobacteria, and Proteobacteria, together they constitute more than 95% of the gut microbiome. Previous studies have shown that higher proportions of Firmicutes and lower proportions of Actinobacteria and Proteobacteria are commonly observed in CRC patients compared to the gut microbiome of healthy subjects. Currently, quantitative microbiome analysis for investigating microbiome dysbiosis as a biomarker in CRC is performed via 16S rRNA sequencing that is costly, time-consuming, and difficult to scale-up. An implementation of SAMBA to rapidly perform quantitative analysis of these major phyla would be a valuable tool for routine clinical implementation of microbiome assay.

### In Silico Validation and Theoretical Consideration

The present disclosure first develops SMB probes that can enable classification of 16S rDNA sequence at the phylum level. The inventors of the present disclosure analysed 16S rDNA next generation sequencing results (V3-V4 region of the 16S rRNA gene) from stool samples of healthy subjects (n=25) and CRC patients (n=38) admitted to the National University Hospital in Singapore. Taxonomy of sequences was identifies using the SILVA bacteria database. Bioinformatics analysis showed that intra-phylum sequences are more similar than inter-phylum sequences.

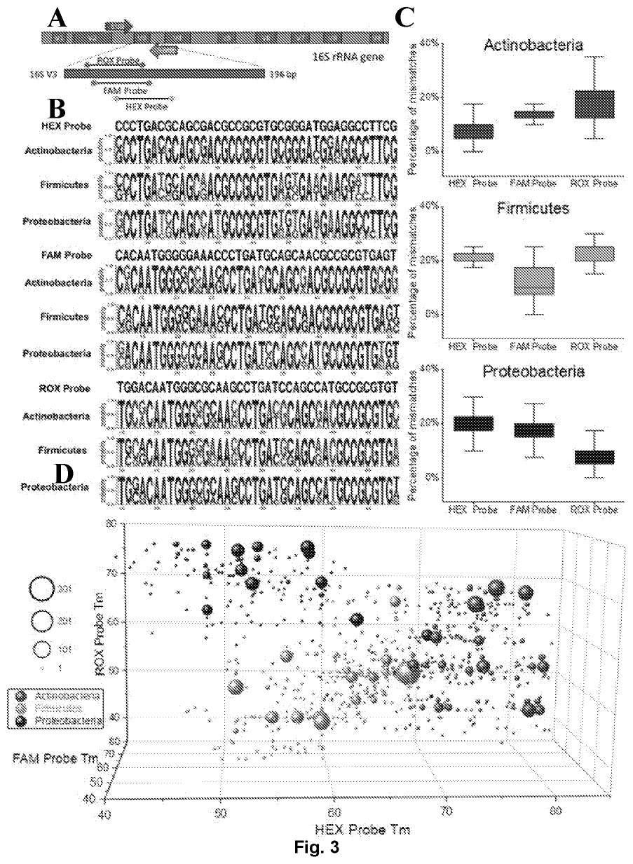

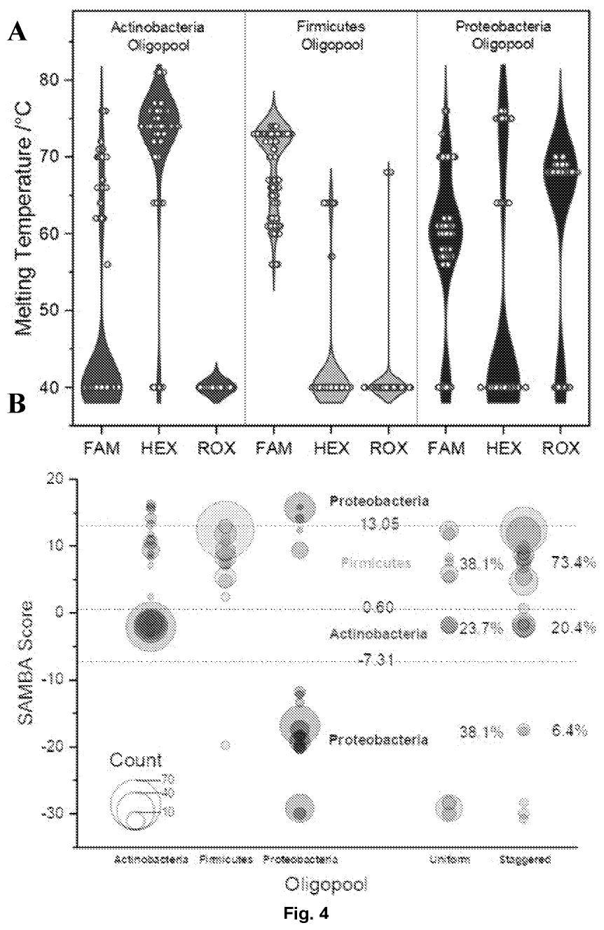

The present disclosure developed a bioinformatic pipeline to design an optimal set of 40 nucleotide-long SMB probes for determination of the phylum of specific 16S rRNA gene sequence. Using a sliding-window approach, the inventors of the present disclosure determined at each position probe sequences that would have the greatest affinity and least intra-phylum variability with a target phylum. The inventors then chose a probe sequence among these sequences that is able to best distinguish the target phylum from non-target phylum. Using this approach, SMB probes can be identified, that when used simultaneously, allows for accurate determination of the phylum. The locations of these probes in the V3 region (FIG. 3A). In FIG. 3B, the present disclosure showed that each of the SMB probes is highly complementary to the sequences in a target phylum but have significantly more mismatches to sequences in a non-target phylum at specific nucleotide positions. The percentage of mismatch of each SMB probe was plotted to corresponding sequences in the NGS results (FIG. 3C) and showed that sequences from a target phylum are represented by a characteristic affinity pattern (mismatches) to the three SMB probes that allows them to be identified.

The availability of only three fluorescence channels in the commercial dPCR platform limits the number of SMB probes that can be used. The inventors of the present disclosure prioritized the design of SMB probes to be able to distinguish three of the dominant phyla Firmicutes, Proteobacteria, and Actinobacteria as changes in their proportions is reported in CRC. 16S rDNA sequence of the other dominant phylum, Bacteriodetes, is significantly different from the three phyla targeted here. Bacteriodetes 16S rDNA have very high number of mismatches to FAM, HEX and ROX probes (FIG. 4). They would be effectively excluded from SAMBA measurements by virtue of the probe design.

Next, the inventors of the present disclosure estimated the combined Tm profiles of 16S rDNA sequence corresponding to different phyla when hybridized with the three designed SMB probes. The DINAMelt software is used to estimate the thermodynamic properties of the hybridization between the target sequence and SMB probes (parameters: sodium salt at 10 mM, magnesium salt at 1 mM, strand concentration at 0.01 μM). The theoretically predicted Tm values of the gut microbiome (from NGS) are visualized in a 3D scatter plot (FIG. 3D), with while the phylum information represented by different colors and the abundance of particular bacteria species represented by the size of the bubbles. It is observed that sequences from different phyla occupy distinct regions in the 3D Tm space. This provides the theoretical basis for the application of SAMBA for gut microbiome profiling.

### In Vitro Validation

The inventors of the present disclosure proceed to perform experimental validation to the theoretical prediction that DNA from the same phylum could have specific Tm profile that separate one from another, yet species from the same phylum can be defined by a range of Tm's. To do so, the inventors prepared pools of synthetic oligonucleotides corresponding to a panel of most abundant 16S rDNA gene sequence from each of the three phyla Firmicutes (24 sequences), Actinobacteria (21 sequences) and Proteobacteria (11 sequences) (Table 5). The inventors first performed SAMBA assay on the Actinobacteria pool, Firmicutes pool and Proteobacteria pool separately and obtained the Tm profiles in three fluorescence channels for each positive compartments.

TABLE 5
DNA sequences of the synthetic oligonucleotides corresponding
to a panel of most abundant 16S rDNA gene sequence from each of the three
phyla Firmicutes (24 sequences), Actinobacteria (21 sequences) and
Proteobacteria (11 sequences).
Actinobacteria
ACTCCTACGGGAGGCAGCAGGGGGAATATTGCACAATGGGCGCAAGCCTGATGCAG
CGACGCCGCGTGCGGGATGACGGCCTTCGGGTTGTAAACCGCTTTTGACTGGGAGC
AAGCCCTTCGGGGTGAGTGTACCTTTCGAATAAGCTCCGGCTAACTACGTGCCAGCA
GCCGCGGTAAT (SEQ ID NO: 10)
ACTCCTACGGGAGGCAGCAGGGGGAATCTTGCGCAATGGGGGGAACCCTGACGCA
GCGACGCCGCGTGCGGGACGGAGGCCTTCGGGTCGTAAACCGCTTTCAGCAGGGA
AGAGTCAAGACTGTACCTGCAGAAGAAGCCCCGGCTAACTACGTGCCAGCAGCCGC
GGTAAT (SEQ ID NO: 11)
ACTCCTACGGGAGGCAGCAGGGGGAATATTGCACAATGGGCGCAAGCCTGATGCAG
CGACGCCGCGTGAGGGATGGAGGCCTTCGGGTTGTAAACCTCTTTTATCGGGGAAC
AAGCGAGAGTGAGTTTACCCGTTGAATAAGCACCGGCTAACTACGTGCCAGCAGCC
GCGGTAAT (SEQ ID NO: 12)
ACTCCTACGGGAGGCAGCAGGGGGAATATTGCACAATGGGCGCAAGCCTGATGCAG
CGACGCCGCGTGAGGGATGGAGGCCTTCGGGTTGTAAACCTCTTTTATCGGGGAGC
AAGCCTTCGGGTGAGTGTACCTTTCGAATAAGCGCCGGCTAACTACGTGCCAGCAG
CCGCGGTAAT (SEQ ID NO: 13)
ACTCCTACGGGAGGCAGCAGGGGGAATATTGCACAATGGGCGCAAGCCTGATGCAG
CGACGCCGCGTGCGGGATGGAGGCCTTCGGGTTGTAAACCGCTTTTGATCGGGAGC
AAGCCCTTCGGGGTGAGTGTACCCTTCGAATAAGCACCGGCTAACTACGTGCCAGC
AGCCGCGGTAAT (SEQ ID NO: 14)
ACTCCTACGGGAGGCAGCAGGGGGAATCTTGCGCAATGGGCGGAAGCCTGACGCA
GCGACGCCGCGTGCGGGAGGAAGGCCCTCGGGTCGTAAACCGCTTTCAGCAGGGA
CGAGGCCGCAAGGTGACGGTACCTGCAGAAGAAGCCCCGGCTAACTACGTGCCAG
CAGCCGCGGTAAT (SEQ ID NO: 15)
ACTCCTACGGGAGGCAGCAGGGGGAATTTTGCGCAATGGGGGAAACCCTGACGCAG
CAACGCCGCGTGCGGGACGACGGCCTTCGGGTTGTAAACCGCTTTCAGCAGGGAAG
AAATTCGACGGTACCTGCAGAAGAAGCTCCGGCTAACTACGTGCCAGCAGCCGCGG
TAAT (SEQ ID NO: 16)
ACTCCTACGGGAGGCAGCAGGGGGAATATTGCACAATGGGCGCAAGCCTGATGCAG
CGACGCCGCGTGCGGGATGACGGCCTTCGGGTTGTAAACCTCTTTTGTTAGGGAGC
AAGGCACTTTGTGTTGAGTGTACCTTTCGAATAAGCACCGGCTAACTACGTGCCAGC
AGCCGCGGTAAT (SEQ ID NO: 17)
ACTCCTACGGGAGGCAGCAGGGGGAATCTTGCGCAATGGGGGCAACCCTGACGCA
GCGACGCCGCGTGCGGGACGAAGGCCTTCGGGTCGTAAACCGCTTTCAGCAGGGA
CGAGGCAAGACGGTACCTGCAGAAGAAGCCCCGGCTAACTACGTGCCAGCAGCCG
CGGTAAT (SEQ ID NO: 18)
ACTCCTACGGGAGGCAGCAGGGGGAATCTTGCGCAATGGGGGGAACCCTGACGCA
GCGACGCCGCGTGCGGGATGGAGGCCTTCGGGTCGTGAACCGCTTTCAGCAGGGA
CGAGTCTGACGGTACCTGCAGAAGAAGCCCCGGCTAACTACGTGCCAGCAGCCGCG
GTAAT (SEQ ID NO: 19)
ACTCCTACGGGAGGCAGCAGGGGGAATCTTGCGCAATGGGCGAAAGCCTGACGCA
GCGACGCCGCGTGCGGGACGAAGGCCTTCTGGTCGTAAACCGCTTTCAGCAGGGA
CGAGGGGGAGACCTGACGGTACCTGCAGAAGAAGCCCCGGCTAACTACGTGCCAG
CAGCCGCGGTAAT (SEQ ID NO: 20)
ACTCCTACGGGAGGCAGCAGGGGGAATCTTGCGAAATGGGGGCAACCCTGACGCA
GCGACGCCGCGTACGGGACGAAGGCCTTCGGGTCGTAAACCGCTTTCAGCAGGGA
CGAGGCCGGAAGGTGACGGTACCTGCAGAAGAAGCCCCGGCTAACTACGTGCCAG
CAGCCGCGGTAAT (SEQ ID NO: 21)
ACTCCTACGGGAGGCAGCAGGGGGAATATTGCACAATGGGCGCAAGCCTGATGCAG
CGACGCCGCGTGCGGGATGACGGCCTTCGGGTTGTAAACCGCTTTCGATCGGGAGC
AAGCCTTCGGGTGAGTGTACCTTTCGAATAAGCACCGGCTAACTACGTGCCAGCAGC
CGCGGTAAT (SEQ ID NO: 22)
ACTCCTACGGGAGGCAGCAGGGGGAATTTTGCGCAATGGGGGCAACCCTGACGCA
GCAACGCCGCGTGCGGGATGACGGCCTTCGGGTTGTAAACCGCTTTCAGCAGGGAA
GACCGACGACGGTACCTGCAGAAGAAGCCCCGGCTAACTACGTGCCAGCAGCCGC
GGTAAT (SEQ ID NO: 23)
ACTCCTACGGGAGGCAGCAGGGGGAAGGTTGCACAATGGGCGAAAGCCTGATGCA
GCGACGCCGCGTGCGGGAAGGAGGCCCTCGGGTCGTAAACCGCTTTCAGCAGGGA
CGAGGCCGCGAGGTGACGGTACCTGCAGAAGAAGCCCCGGCTAACTACGTGCCAG
CAGCCGCGGTAAT (SEQ ID NO: 24)
ACTCCTACGGGAGGCAGCAGGGGGAATCTTGCGCAATGGGGGCAACCCTGACGCA
GCGACGCCACGTGCGGGATGGAGGCCTTCGGGTCGTAAACCGCTTTCAGCAGGGA
CGAGTCAAGACTGTACCTGCAGAAGAAGCCCCGGCTAACTACGTGCCAGCAGCCGC
GGTAAT (SEQ ID NO: 25)
ACTCCTACGGGAGGCAGCAGGGGGAATTTTGCGCAATGGGGGCAACCCTGACGCA
GCAACGCCGCGTGCGGGACGAAGGCGTCCGCGTCGTAAACCGCTTTCAGCGGGGA
ACACCTAACGAGGGTACCCGCAGAAGAAGCCCCGGCTAAATACGTGCCAGCAGCCG
CGGTAAT (SEQ ID NO: 26)
ACTCCTACGGGAGGCAGCAGGGGGAATATTGCGCAATGGGGGGAACCCTGACGCA
GCGACGCCGCGTGCGGGACGGAGGCCTTCGGGTCGTAAACCGCTTTCAGCAGGGA
AGACATAGACGGTACCTGCAGAAGAAGCTCCGGCTAACTACGTGCCAGCAGCCGCG
GTAAT (SEQ ID NO: 27)
ACTCCTACGGGAGGCAGCAGGGGGAATCTTGCGCAATGGGGGCAACCCTGACGCA
GCGACGCCGCGTGCGGGATGGAGGCCTTCGGGTCGTAAACCGCTTTCAGCAGGGA
CGAGCCAAGACGGTACCTGCAGAAGAAGCCCCGGCTAACTACGTGCCAGCAGCCGC
GGTAAT (SEQ ID NO: 28)
ACTCCTACGGGAGGCAGCAGGGGGAATATTGCACAATGGGCGCAAGCCTGATGCAG
CGACGCCGCGTGCGGGATGGAGGCCTTCGGGTTGTAAACCGCTTTTGTTCAAGGGC
AAGGCACGGTTTCGGCCGTGTTGAGTGGATTGTTCGAATAAGCACCGGCTAACTACG
TGCCAGCAGCCGCGGTAAT (SEQ ID NO: 29)
ACTCCTACGGGAGGCAGCAGGGGGAATATTGCACAATGGGGGCAACCCTGACGCAG
CGACGCCGCGTGCGGGACGGAGGCCTTCGGGTCGTAAACCGCTTTCAGCAGGGAA
GAGACAAGACTGTACCTGCAGAAGAAGCCCCGGCTAACTACGTGCCAGCAGCCGCG
GTAAT (SEQ ID NO: 30)
Firmicutes
ACTCCTACGGGAGGCAGCAGGGGGAATATTGCACAATGGGGGAAACCCTGATGCAG
CGACGCCGCGTGAAGGAAGAAGTATCTCGGTATGTAAACTTCTATCAGCAGGGAAGA
CAGTGACGGTACCTGACTAAGAAGCCCCGGCTAACTACGTGCCAGCAGCCGCGGTA
AT (SEQ ID NO: 31)
ACTCCTACGGGAGGCAGCAGGGGGAATCTTCCGCAATGGACGAAAGTCTGACGGAG
CAACGCCGCGTGAACGATGACGGCCTTCGGGTTGTAAAGTTCTGTTATACGGGACG
AATGGTACGACGGTCAATACCCGTCGTAAGTGACGGTACCGTAAGAGAAAGCCACG
GCTAACTACGTGCCAGCAGCCGCGGTAAT (SEQ ID NO: 32)
ACTCCTACGGGAGGCAGCAGAGGGAATCTTCGGCAATGGGGGCAACCCTGACCGA
GCAACGCCGCGTGAGTGAAGAAGGTTTTCGGATCGTAAAGCTCTGTTGTAAGTCAAG
AACGAGTGTGAGAGTGGAAAGTTCACACTGTGACGGTAGCTTACCAGAAAGCGACG
GCTAACTACGTGCCAGCAGCCGCGGTAAT (SEQ ID NO: 33)
ACTCCTACGGGAGGCAGCAGGGGGAATATTGCACAATGGGGGAAACCCTGATGCAG
CGACGCCGCGTGAGCGATGAAGTATTTCGGTATGTAAAGCTCTATCAGCAGGGAAGA
TAGTGACAGTACCTGACTAAGAAGCCCCGGCTAACTACGTGCCAGCAGCCGCGGTA
AT (SEQ ID NO: 34)
ACTCCTACGGGAGGCAGCAGGGGGAATCTTCCGCAATGGGCGAAAGCCTGACGGA
GCAACGCCGCGTGAACGATGAAGGTCTTAGGATCGTAAAGTTCTGTTGTTAGGGACG
AAGGGTAAGGATAATAATAAGGTTTTTATTTGACGGTACCTAACGAGGAAGCCACGG
CTAACTACGTGCCAGCAGCCGCGGTAAT (SEQ ID NO: 35)
ACTCCTACGGGAGGCAGCAGGGGGAATATTGCACAATGGGGGAAACCCTGATGCAG
CGACGCCGCGTGGAGGAAGAAGGTCTTCGGATTGTAAACTCCTGTTGTTGAGGAAG
ATAATGACGGTACCCAACAAGGAAGTGACGGCTAACTACGTGCCAGCAGCCGCGGT
AAA (SEQ ID NO: 36)
ACTCCTACGGGAGGCAGCAGGGGGAATCTTCCGCAATGGACGAAAGTCTGACGGAG
CAACGCCGCGTGAGTGATGAAGGATTTCGGTCTGTAAAGCTCTGTTGTTTATGACGA
ACGTGCAGTGTGTGAACAATGCAATGCAATGACGGTAGTAAACGAGGAAGCCACGG
CTAACTACGTGCCAGCAGCCGCGGTAAT (SEQ ID NO: 37)
ACTCCTACGGGAGGCAGCAGGGGGAATATTGCACAATGGGGGAAACCCTGATGCAG
CGACGCCGCGTGAGCGAAGAAGTATTTCGGTATGTAAAGCTCTATCAGCAGGGAAG
ATAATGACGGTACCTGACTAAGAAGCACCGGCTAAATACGTGCCAGCAGCCGCGGT
AAT (SEQ ID NO: 38)
ACTCCTACGGGAGGCAGCAGGGGGAATCTTCCGCAATGGACGAAAGTCTGACGGAG
CAACGCCGCGTGAACGATGACGGCCTTCGGGTTGTAAAGTTCTGTTATATGGGACGA
ACAGGATAACGGTTAATACCCGATATCCCTGACGGTACCGTAAGAGAAAGCCACGGC
TAACTACGTGCCAGCAGCCGCGGTAAT (SEQ ID NO: 39)
ACTCCTACGGGAGGCAGCAGGGGGAATCTTCCGCAATGGACGAAAGTCTGACGGAG
CAACGCCGCGTGAGTGATGAAGGTCTTCGGATTGTAAAACTCTGTTGTTAGGGACGA
AAGCACCGTGTTCGAACAGGTCATGGTGTTGACGGTACCTAACGAGGAAGCCACGG
CTAACTACGTGCCAGCAGCCGCGGTAAA (SEQ ID NO: 40)
ACTCCTACGGGAGGCAGCAGGGGGAATATTGCACAATGGGGGAAACCCTGATGCAG
CGACGCCGCGTGAGCGAAGAAGTATTTCGGTATGTAAAGCTCTATCAGCAGGGAAG
AAAATGACGGTACCTGACTAAGAAGCCCCGGCTAACTACGTGCCAGCAGCCGCGTG
TAAT (SEQ ID NO: 41)
ACTCCTACGGGAGGCAGCAGGGGGAATATTGCACAATGGGGGAAACCCTGATGCAG
CGACGCCGCGTGAGCGATGAAGTACTTCGGTATGTAAAGCTCTATCAGCAGGGAAG
AAAATGACGGTACCTGACTAAGAAGCACCGGCTAAATACGTGCCAGCAGCCGCGGT
AAT (SEQ ID NO: 42)
ACTCCTACGGGAGGCAGCAGGGGGAATATTGCACAATGGGGGAAACCCTGATGCAG
CGACGCCGCGTGAAGGAAGAAGTATCTCGGTATGTAAACTTCTATCAGCAGGGAAGA
AGAAATGACGGTACCTGACTAAGAAGCACCGGCTAACTACGTGCCAGCAGCCGCGG
TAAT (SEQ ID NO: 43)
ACTCCTACGGGAGGCAGCAGGGGGGACATTGCACAATGGGGGAAACCCTGATGCA
GCGACGCCGCGTGGAGGAAGAAGGTTTTCGGATTGTAAACTCCTGTCGTTAGGGAC
GATAATGACGGTACCTAACAAGAAAGCACCGGCTAACTACGTGCCAGCAGCCGCGG
TAAA (SEQ ID NO: 44)
ACTCCTACGGGAGGCAGCAGGGGGAATATTGCACAATGGGGGAAACCCTGATGCAG
CGACGCCGCGTGAGTGAAGAAGTATCTCGGTATGTAAAGCTCTATCAGCAGGGAAG
AAAATGACGGTACCTGACTAAGAAGCCCCGGCTAACTACGTGCCAGCAGCCGCGGT
AAT (SEQ ID NO: 45)
ACTCCTACGGGAGGCAGCAGGGGGAATCTTCCGCAATGGACGAAAGTCTGACGGAG
CAACGCCGCGTGAGTGAAGAAGGTCTTCGGACTGTAAAACTCTGTTGTTAGGGACGA
AAGCAGTTATGAATAACAAGTGTAGCTGTTGACGGTACCTGACGAGGAAGCCACGGC
TAACTACGTGCCAGCAGCCGCGGTAAT (SEQ ID NO: 46)
ACTCCTACGGGAGGCAGCAGGGGGAATCTTCCGCAATGGACGAAAGTCTGACGGAG
CAACGCCGCGTGAGTGATGACGGCCTTCGGGTTGTAAAGCTCTGTTAATCGGGACG
AAAGGTCCTCTTGCGAATAGTTAGAGGAATTGACGGTACCGGAATAGAAAGCCACGG
CTAACTACGTGCCAGCAGCCGCGGTAAT (SEQ ID NO: 47)
ACTCCTACGGGAGGCAGCAGGGGGAATATTGCACAATGGGGGAAACCCTGATGCAG
CGACGCCGCGTGAGCGAAGAAGTATTTCGGTATGTAAAGCTCTATCAGCAGGGAAG
AAGAAATGACGGTACCTGACTAAGAAGCACCGGCTAAATACGTGCCAGCAGCCGCG
GTAAT (SEQ ID NO: 48)
ACTCCTACGGGAGGCAGCAGGGGGAATATTGCACAATGGGGGAAACCCTGATGCAG
CGACGCCGCGTGAAGGAAGAAGTATCTCGGTATGTAAACTTCTATCAGCAGGGAAGA
TAGTGACGGTACCTGACTAAGAAGCCCCGGCTAACTTACGTGCCAGCAGCCGCGGT
AAT (SEQ ID NO: 49)
ACTCCTACGGGAGGCAGCAGGGGGAATCTTGCGCAATGGGGGCAACCCTGACGCA
GCAACGCCGCGTGAAGGATGAAGGTTTTCGGATTGTAAACTTCTTTTATTAAGGACG
AAAATTGACGGTACTTAATGAATAAGCTCCGGCTAACTACGTGCCAGCAGCCGCGGT
AAT (SEQ ID NO: 50)
ACTCCTACGGGAGGCAGCAGGGGGAATATTGCACAATGGAGGAAACTCTGATGCAG
CGACGCCGCGTGAAGGATGAAGTATTTCGGTATGTAAACTTCTATCAGCAGGGAAGA
AAATGACGGTACCTGACTAAGAAGCACCGGCTAAATACGTGCCAGCAGCCGCGGTA
AT (SEQ ID NO: 51)
ACTCCTACGGGAGGCAGCAGGGGGAATATTGCACAATGGGGGAAACCCTGATGCAG
CGACGCCGCGTGAAGGAAGAAGTATCTCGGTTTGTAAACTTCTATCAGCAGGGAAGA
TAATGACGGTACCTGACTAAGAAGCCCCGGCTAACTACGTGCCAGCAGCCGCGGTA
AT (SEQ ID NO: 52)
ACTCCTACGGGAGGCAGCAGGGGGAATCTTGCGCAATGGGGGAAACCCTGATGCAG
CGACGCCGCGTGAAGGAAGAAGTATCTCGGTATGTAAACTTCTATCAGCAGGGAAGA
TAGTGACGGTACCTGACTAAGAAGCCCCGGCTAACTACGTGCCAGCAGCCGCGGTA
AT (SEQ ID NO: 53)
ACTCCTACGGGAGGCAGCAGAGGGAATCTTCGGCAATGGGGGCAACCCTGACCGA
GCAACGCCGCGTGAGTGAAGAAGGTTTTCGGATCGTAAAGCTCTGTTGTAAGAGAAG
AACGAGTGTGAGAGTGGAAAGTTCACACTGTGACGGTAACTTACCAGAAAGGGACG
GCTAACTACGTGCCAGCAGCCGCGGTAAT (SEQ ID NO: 54)
Proteobacteria
ACTCCTACGGGAGGCAGCAGGGGGAATATTGCACAATGGGCGCAAGCCTGATGCAG
CCATGCCGCGTGTATGAAGAAGGCCTTCGGGTTGTAAAGTACTTTCAGCGGGGAGG
AAGGGAGTAAAGTTAATACCTTTACTCATTGACGTTACCCGCAGAAGAAGCCCCGGC
TAACTACGTGCCAGCAGCCGCGGTAAT (SEQ ID NO: 55)
ACTCCTACGGGAGGCAGCAGGGGGAATTTTGGACAATGGGCGAAAGCCTGATCCAG
CAATGCCGCGTGTGTGAAGAAGGCCTTCGGGTTGTAAAGCACTTTTGTCCGGAAAGA
AATCCTTGGCTCTAATACAGTCGGGGGATGACGTTACCGGAAGAATAAGCACCGGCT
AACTACGTGCCAGCAGCCGCGGTAAT (SEQ ID NO: 56)
ACTCCTACGGGAGGCAGCAGGGGGAATATTGCACAATGGGCGCAAGCCTGATGCAG
CCATGCCGCGTGTGTGAAGAAGGCCTTCGGGTTGTAAAGCACTTTCAGCGGGGAGG
AAGGCGGTGAGGTTAATAACCTTGGCGATTGACGTTACCCGCAGAAGAAGCACCGG
CTAACTCCGTGCAGCAGCCGGGGTAAT (SEQ ID NO: 57)
ACTCCTACGGGAGGCAGCAGGGGGAATATTGGACAATGGGGGCAACCCTGATCCAG
CCATGCCGCGTGAGTGATGAAGGCCCTAGGGTTGTAAAGCTCTTTTGTGCGGGAAG
ATAATGACGGTACCGCAAGAATAAGCCCCGGCTAACTTCGTGCCAGCAGCCGCGGT
AAT (SEQ ID NO: 58)
ACTCCTACGGGAGGCAGCAGGGGGAATATTGGACAATGGGCGCAAGCCTGATCCAG
CCATGCCGCGTGGGTGAAGAAGGCCTTCGGGTTGTAAAGCCCTTTTGTTCGGGAAG
AAATCGTCTGGGCTAATACCCCGGGCGGATGACGGTACCGGAAGAATAAGCACCGG
CTAACTTCGTGCCAGCAGCCGCGGTAAT (SEQ ID NO: 59)
ACTCCTACGGGAGGCAGCAGGAGGAATATTGCACAATGGGCGCAAGCCTGATCCAG
CTATTCCGCGTGTGGGATGAAGGCCCTCGGGTTGTAAACCACTTTTGTAGAGAACGA
AAAGACACCTTCGAATAAAGGGTGTTGCTGACGGTACTCTAAGAATAAGCACCGGCT
AACTACGTGCCAGCAGCCGCGGTAAT (SEQ ID NO: 60)
ACTCCTACGGGAGGCAGCAGGGGGAATATTGCACAATGGGGGAAACCCTGACGCAG
CGACGCCGCGTGAGGGATGAAGGTTCTCGGATCGTAAACCTCTGTCAGGGGGGAAG
AAACCCCCTCGTGTGAATAATGCGAGGGCTTGACGGTACCCCCAAAGGAAGCACCG
GCTAACTCCGTGCCAGCAGCCGCGGTAAT (SEQ ID NO: 61)
ACTCCTACGGGAGGCAGCAGGGGGAATTTTGGACAATGGGGGCAACCCTGATCCAG
CCATGCCGCGTGCAGGATGAAGGTCTTCGGATTGTAAACTGCTTTTGTCAGGGACGA
AAAGGGATGCGATAACACCGTATTCCGCTGACGGTACCTGAAGAATAAGCACCGGCT
AACTACGTGCCAGCAGCCGCGGTAAT (SEQ ID NO: 62)
ACTCCTACGGGAGGCAGCAGGGGGAATATTGCGCAATGGGGGCAACCCTGACGCA
GCCATGCCGCGTGAATGAAGAAGGCCTTCGGGTTGTAAAGTTCTTTCGGTAGCGAG
GAAGGCATTTAGTTTAATAGACTAGGTGATTGACGTTAACTACAGAAGAAGCACCGG
CTAACTCCGTGCCAGCAGCCGCGGTAAT (SEQ ID NO: 63)
ACTCCTACGGGAGGCAGCAGGGGGAATATTGGACAATGGGGGCAACCCTGATCCAG
CCATGCCGCGTGAGTGATGAAGGCCTTAGGTTTGTAAAGCTCTTTTGTCCGGGACGA
TAATGACGGTACCGGAAGAATAAGCCCCGGCTAACTTCGTGCCAGCAGCCGCGGTA
AT (SEQ ID NO: 64)
ACTCCTACGGGAGGCAGCAGGGGGAATATTGCACAATGGGCGCAAGCCTGATGCAG
CCATGCCGCGTGTATGAAGAAGGCCTTAGGGTTGTAAAGTACTTTCAGCGGGGAGG
AAGGTGATAAGGTTAATACCCTTGTCAATTGACGTTACCCGCAGAAGAAGCACCGGC
TAACTCCGTGCCAGCAGCCGCGGTAAT (SEQ ID NO: 65)

To facilitate comparison of raw Tm profiles of individual rDNA molecules from different samples, the raw Tm values of each probe Tm was rounded to the nearest integer and grouped trios of Tm's with the same integer values into Operational Taxonomic Units (OTUs). SAMBA OTUs are related to conventional NGS OTUs (16S rDNA sequences sharing high degree of sequence similarities) in that both are units that are determined by sequence similarity. The study results yielded 54 unique SAMBA OTUs.

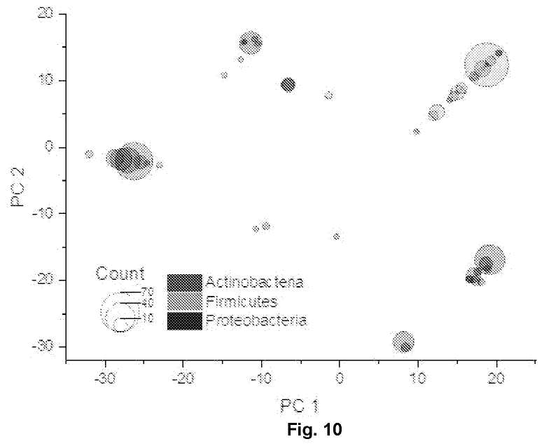

Violin plots of the Tm values was constructed in three fluorescence channels for the phylum oligonucleotide pools (FIG. 4A). The inventors observed distinctive inter-phylum differences in Tm profiles, in a manner that correlates negatively with the degree of probe mismatch with species in the phylum (FIG. 3C), confirming the intended outcome of the probe design. Next, the inventors proceed to determine an appropriate SAMBA Score to distinguish among the different taxonomic group. The inventors performed Principal Component Analysis (PCA) on the multidimensional Tm values of the all the SAMBA OTUs obtained. The results (FIG. 10) showed that while PC 1 provides maximal separation between the Actinobacteria and Firmicutes DNA, PC 2 is able to distinguish among Actinobacteria, Firmicutes and Proteobacteria. Thus the inventors can define the SAMBA Score as PC 2, where

$$SAMBA Score = 0.589 ( T {m}_{F A M} - 57.146 ) + 0.145 ( T {m}_{H E X} - 54.774 ) - 0.795 ( T {m}_{R O X} - 46.490 )$$

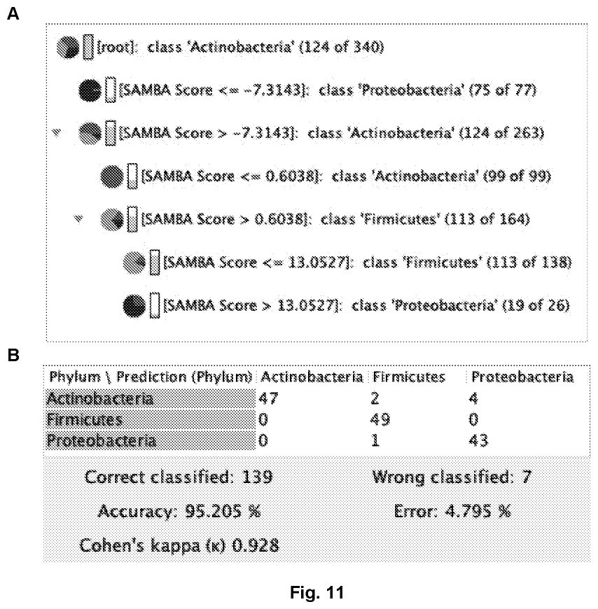

The inventors plotted the SAMBA Scores of the positive compartments in the phylum-specific oligonucleotide pools (FIG. 4B). The inventors trained and validated a decision tree classifier (70% training, 30% validation) to predict the phylum based on SAMBA Score and achieved a high accuracy (95.2%, FIG. 11). With this classifier, compartments with SAMBA Scores between −7.31 and 0.60 will be classified as Actinobacteria, compartments with SAMBA Scores between 0.60 and 13.05 will be classified as Firmicutes, while the rest of the positive compartments will be classified as Proteobacteria.

The inventors next tested the use of SAMBA to quantitative analysis of mixed samples containing DNA from different phyla. The inventors performed SAMBA assay on two mixtures of equal and staggered proportion of the phylum-specific oligonucleotide pools (Actinobacteria:Firmicutes:Proteobacteria=33%:33%:33% and 27%:67%:5%). Using the decision tree classifier developed earlier, the inventors calculated the phylum proportions in SAMBA assay to be 24%:38%:38% and 20%:73%:5% for the equal and staggered mixtures respectively, demonstrating that SAMBA assay can yield accurate quantitative analysis of microbiome compositions, in samples that contain up to 56 different synthetic DNA species.

### SAMBA Accurately Identifies Microbiome Composition in Clinical Samples

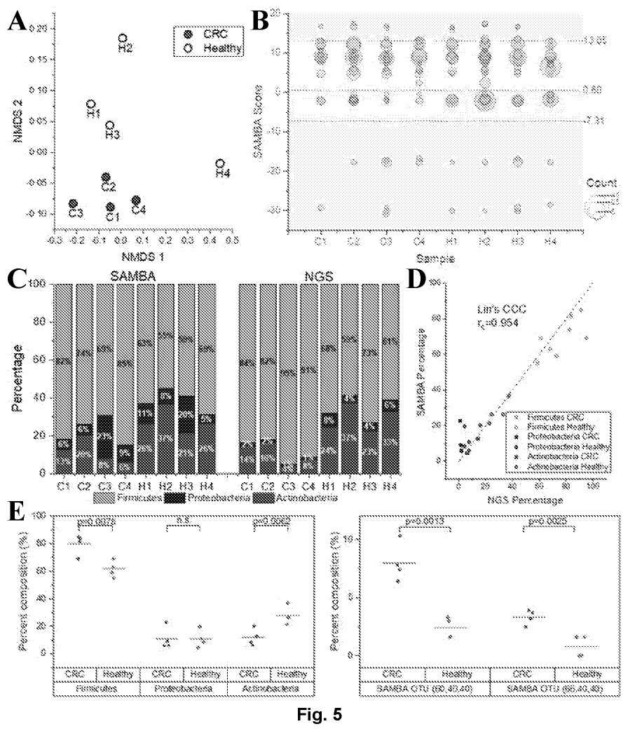

To demonstrate the utility of SAMBA for microbiome analysis in clinical setting, the inventors tested SAMBA on 16S rDNA from eight patient stool samples (C1 to C4 for CRC samples and H1 to H4 for healthy samples) that were subjected to concurrent NGS microbiome analysis in the 16S V3 region. As before, the inventors aggregated measurements into SAMBA OTUs to facilitate microbiome abundance analysis. To analyze the similarity in microbiome composition among the different samples, the inventors performed Principal Coordinate Analysis (PCoA) based on Bray-Curtis dissimilarity among the SAMBA OTUs in different samples (FIG. 5A). The results showed that fecal microbiome of healthy subjects and CRC patients can be separated in the NMDS plot, suggesting that compositional differences may exist between the microbiome composition of healthy and disease samples.

Next, the inventors plotted the SAMBA Scores for the clinical samples (FIG. 5B). The results showed that while the microbiome samples shared many SAMBA OTUs, there are observable differences in their abundance and presence of a number of sample-specific SAMBA OTUs. This is consistent with current understanding of gut microbiome where there is often a shared core microbiome but also distinct bacteria communities associated with particular individuals. The inventors next assigned each SAMBA Score measurement to a phylum based on the previously defined classifier, and calculated the relative proportions of each of the phylum in all the clinical samples (FIG. 5C). The matching NGS analysis of the microbiome composition is also presented for comparison. It is observed that the microbiome composition determined by SAMBA assay highly resembles the composition determined by NGS analysis. The inventors compared the SAMBA measured phyla percentages for all eight samples with the phyla percentage measured by NGS (FIG. 5D). The bacteria composition measured by SAMBA is highly concordant with NGS, with a Lin's Concordance Correlation Coefficient r_{c} of 0.95. These results demonstrate that the SAMBA assay is a robust and accurate method of quantifying the phylum composition of human stool samples.

The inventors observed in SAMBA assay an increase in Firmicutes and decrease in Actinobacteria for CRC patients compared to healthy subjects. This difference is statistically significant in two-tailed t-test (FIG. 5E) and agrees with similar observation previously reported in literature¹⁹. Although the inventors have focused on phylum classification in this manuscript due to its known clinical significance and ease of validation, SAMBA is capable of quantitative analysis of microbiome composition at much higher resolution. The inventors compared the relative abundance of SAMBA OTUs between the samples from CRC patients and healthy subjects, and found two SAMBA OTUs, 60_{FAM}40_{HEX}40_{ROX} and 66_{FAM}40_{HEX}40_{ROX} belonging to the Firmicutes phylum that are significantly higher (two tailed t-test) in CRC patients compared to healthy subjects (FIG. 5F). These results demonstrate potential biomarker discovery and measurement with quantitative analysis at the SAMBA OTU levels. Significantly, these highly quantitative results are obtained from a single SAMBA measurement that can be performed much more rapidly and simply compared to NGS approaches. These results lay the foundation for future validation of this method on a larger cohort of samples as a scalable diagnostics method.

There is increasing utility of using microbiome composition to use it as a biomarker for prognostic disease testing of CRC, or to look at the usage of prebiotics and probiotics as treatment for diseases like inflammatory bowel disorder and diabetes. However, it is still challenging to scale up NGS for routine clinical implementation due to the complicated workflow, high cost and long turnaround time. There is a lack of a simple microbiome diagnostics technologies that can rival the multiplexing and quantitative capability of NGS.

Digital PCR is highly attractive as a rapid and highly quantitative platform for profiling microbial species. However, the lack of multiplexing capability in dPCR so far has precluded it from being used for microbiome analysis. Although there have been efforts to enable discrimination of bacteria species, most notably through HRM analysis in dPCR, they are limited to distinguishing bacteria with very different intrinsic GC contents. So far, these platforms have been demonstrated on selected mixtures of bacteria species that are distinguishable with HRM. There is, however, no generalizable relationship that would allow dHRM to identify bacteria species or taxons from a complex microbiome community.

This SAMBA assay is the first demonstration of a multicolor probe-based dPCR assay that has a superior multiplexing capability which is useful for processing and analysing a complex sample such as the microbiome of human stool samples. The combination of PCR, single-molecule partition, and melting curve analysis allows for a rapid, cost effective and quantitative assay. SAMBA assay is easy to perform, high throughput (up to 48 samples per chip in current setting), rapid (<4 hours), requires a low amount of sample (<1 ng), and thus represents a huge improvement as a microbiome diagnostics method for large-scale clinical studies.

In this disclosure, the inventors have shown that SAMBA achieves the highest multiplexing capability (16-plex) reported to date for digital PCR. Absolute quantification of mixed bacteria sample is demonstrated, and the inventors have developed the notion of SAMBA Score to facilitate easy classification of different bacteria in the mixture. The present disclosure has shown that using three carefully designed SMB probes, the phylum information on highly complex gut microbiome samples can be accurately determined. Extensive validation of this platform was performed using synthetic oligonucleotides, and finally showed that the platform as described herein is able to measure microbiome composition from stool samples in healthy and CRC patients, with results closely matching concurrent NGS analysis of the same samples.

The ability to rapidly identifying the phylum compositions changes using SAMBA is already potentially very attractive due to many studies linking higher level taxonomy changes in microbiome to specific states of health. The inventors also showed that temperature based OTU-level analysis is possible with SAMBA and have found specific SAMBA OTUs that are significantly different between microbiome of healthy subjects and cancer patients. Thus, SAMBA may have utility for polymicrobial profiling with much higher taxonomic resolution. The inventors also note that multiplexing capability of SAMBA can be further improved with use of additional probes to give signals at different fluorescence channels. This is on the horizon with the development of dPCR platforms with increased throughput and fluorescence channels.

In summary, SAMBA combines the superior multiplexing capability of SMB and the rapid, cost-effective, and quantitative aspect of dPCR to analyze complex samples such as the human microbiome. With dPCR assays becoming more common in the clinical setting, SAMBA provides an alternative method of performing microbiome diagnostic test.

### REFERENCES

1. Sobhani, I. et al. Microbial Dysbiosis in Colorectal Cancer (CRC) Patients. PLoS ONE 6, e16393 (2011).
2. Wu, N. et al. Dysbiosis Signature of Fecal Microbiota in Colorectal Cancer Patients. Microbial Ecology 66, 462-470 (2013).
3. Sokol, H. & Seksik, P. The intestinal microbiota in inflammatory bowel diseases: time to connect with the host. Current Opinion in Gastroenterology 26, 327-331 (2010).
4. Romano, S. et al. Meta-analysis of the Parkinson's disease gut microbiome suggests alterations linked to intestinal inflammation. npj Parkinson's Disease 7, 1-13 (2021).
5. Schlaberg, R. Microbiome Diagnostics. Clinical Chemistry 66, 68-76 (2020).
6. Hwang, K., Shin, S. G., Kim, J. & Hwang, S. Methanogenic profiles by denaturing gradient gel electrophoresis using order-specific primers in anaerobic sludge digestion. Applied Microbiology and Biotechnology 80, 269-276 (2008).
7. De Vrieze, J., Ijaz, U. Z., Saunders, A. M. & Theuerl, S. Terminal restriction fragment length polymorphism is an “old school” reliable technique for swift microbial community screening in anaerobic digestion. Scientific Reports 8, 16818 (2018).
8. Zhong, Q. et al. Multiplex digital PCR: Breaking the one target per color barrier of quantitative PCR. Lab on a Chip (2011) doi:10.1039/cl 1c20126c.
9. El-Hajj, H. H. et al. Use of Sloppy Molecular Beacon Probes for Identification of Mycobacterial Species. Journal of Clinical Microbiology 47, 1190-1198 (2009).
10. Chakravorty, S. et al. Rapid Universal Identification of Bacterial Pathogens from Clinical Cultures by Using a Novel Sloppy Molecular Beacon Melting Temperature Signature Technique. Journal of Clinical Microbiology 48, 258-267 (2010).
11. Sanchez, J. A., Pierce, K. E., Rice, J. E. & Wangh, L. J. Linear-After-The-Exponential (LATE)-PCR: An advanced method of asymmetric PCR and its uses in quantitative real-time analysis. Proceedings of the National Academy of Sciences 101, 1933-1938 (2004).
12. Tang, X., Morris, S. L., Langone, J. J. & Bockstahler, L. E. Simple and effective method for generating single-stranded DNA targets and probes. BioTechniques 40, 759-763 (2006).
13. O'Keefe, C. M. et al. Facile profiling of molecular heterogeneity by microfluidic digital melt. Science Advances 4, eaat6459 (2018).
14. Ley, R. E., Peterson, D. A. & Gordon, J. I. Ecological and Evolutionary Forces Shaping Microbial Diversity in the Human Intestine. Cell 124, 837-848 (2006).
15. Husted, A. S., Trauelsen, M., Rudenko, O., Hjorth, S. A. & Schwartz, T. W. GPCR-Mediated Signaling of Metabolites. Cell Metabolism 25, 777-796 (2017).
16. Antharam, V. C. et al. Intestinal Dysbiosis and Depletion of Butyrogenic Bacteria in Clostridium difficile Infection and Nosocomial Diarrhea. Journal of Clinical Microbiology 51, 2884-2892 (2013).
17. Mudd, J. C. & Brenchley, J. M. Gut Mucosal Barrier Dysfunction, Microbial Dysbiosis, and Their Role in HIV-1 Disease Progression. Journal of Infectious Diseases 214, S58-S66 (2016).
18. Khanna, S. & Tosh, P. K. A Clinician's Primer on the Role of the Microbiome in Human Health and Disease. Mayo Clinic Proceedings 89, 107-114 (2014).
19. Gao, Z., Guo, B., Gao, R., Zhu, Q. & Qin, H. Microbiota disbiosis is associated with colorectal cancer. Frontiers in Microbiology 6, (2015).
20. Dadkhah, E. et al. Gut microbiome identifies risk for colorectal polyps. BMJ Open Gastroenterology 6, e000297 (2019).
21. Quast, C. et al. The SILVA ribosomal RNA gene database project: Improved data processing and web-based tools. Nucleic Acids Research 41, D590-D596 (2013).
22. Markham, N. R. & Zuker, M. DINAMelt web server for nucleic acid melting prediction. Nucleic Acids Research 33, W577-W581 (2005).
23. Narayanan, V., Peppelenbosch, M. P. & Konstantinov, S. R. Human fecal microbiome-based biomarkers for colorectal cancer. Cancer Prevention Research 7, 1108-1111 (2014).
24. Wasilewski, A., Zielińska, M., Storr, M. & Fichna, J. Beneficial Effects of Probiotics, Prebiotics, Synbiotics, and Psychobiotics in Inflammatory Bowel Disease. Inflammatory Bowel Diseases 21, 1674-1682 (2015).
25. Yoo, J. Y. & Kim, S. S. Probiotics and prebiotics: Present status and future perspectives on metabolic disorders. Nutrients vol. 8 173 (2016).
26. Athamanolap, P. et al. Nanoarray Digital Polymerase Chain Reaction with High-Resolution Melt for Enabling Broad Bacteria Identification and Pheno-Molecular Antimicrobial Susceptibility Test. Analytical Chemistry 91, 12784-12792 (2019).
27. Velez, D. O. et al. Massively parallel digital high resolution melt for rapid and absolutely quantitative sequence profiling. Scientific Reports 7, 1-14 (2017).
28. Whale, A. S., Huggett, J. F. & Tzonev, S. Fundamentals of multiplexing with digital PCR. Biomolecular Detection and Quantification vol. 10 15-23 (2016).
29. Hindson, B. J. et al. High-throughput droplet digital PCR system for absolute quantitation of DNA copy number. Analytical Chemistry 83, 8604-8610 (2011).
30. Schloss, P. D. et al. Introducing mothur: Open-source, platform-independent, community-supported software for describing and comparing microbial communities. Applied and Environmental Microbiology 75, 7537-7541 (2009).
31. Rognes, T., Flouri, T., Nichols, B., Quince, C. & Mahé, F. VSEARCH: A versatile open source tool for metagenomics. PeerJ 2016, e2584 (2016).

### Applications

Embodiments of the methods disclosed herein provide a high throughput approach that has superior multiplexing capability for rapidly identifying the different microbial (such as bacterial) phylum and quantifying its proportion. The methods as described herein has been exemplified in colorectal cancer samples and have accurately measured their microbiome composition. The results indicate that the assay as described herein is a useful tool that can allow microbiome to become an important biomarker in disease and treatment planning. The methods as described herein is also transferable to non-medical samples, such as in consumer products to accurately measure their microbiotic composition.

Advantageously, the use of the probes as described herein provides Tm signatures that serve as highly accurate sequence identifier that enables for analysing complex samples such as microbiome. The use of stochastic encapsulation of single 16S rRNA gene copy allows for a mixed sample containing thousands of different 16S rRNA gene targets to be analyzed without interference during the melting curve analysis to obtain thousands of Tm signatures. The feature of enumeration of the positive chambers with Tm signatures allows for the absolute quantification of each Tm signature which allows quantification of the absolute abundance of each bacterial identity.

Even more advantageously, the rapid and cost-effective PCR based approach allows the methods as described herein to be used in large-scale clinical setting.

It will be appreciated by a person skilled in the art that other variations and/or modifications may be made to the embodiments disclosed herein without departing from the spirit or scope of the disclosure as broadly described. For example, in the description herein, features of different exemplary embodiments may be mixed, combined, interchanged, incorporated, adopted, modified, included etc. or the like across different exemplary embodiments. The present embodiments are, therefore, to be considered in all respects to be illustrative and not restrictive.

1. A method of profiling a microbiotic composition of a sample comprising the nucleic acid molecules of the microbiotic composition, the method comprising:
a) partitioning the sample into a sufficient number of partitions such that at least a portion of the partitions comprises no more than one nucleic acid molecule of the microbiotic composition;
b) contacting the partitions of the sample to a plurality of mismatch-tolerant probes that are capable of binding to the nucleic acid molecules or parts thereof under suitable conditions;
c) determining signals generated by each of the plurality of mismatch-tolerant probes in each partition; and
d) establishing the microbiotic composition in the sample based on the signals.

2. The method according to claim 1, wherein the plurality of mismatch-tolerant probes is configured to collectively generate different signal intensity profiles for different groups of nucleic acid molecules.

3. The method according to claim 1, wherein d) comprises computing a melting temperature (Tm) of each of the plurality of mismatch-tolerant probes to obtain a Tm signature for each partition based on the signals generated by each of the plurality of mismatch-tolerant probes in each partition.

4. The method according to claim 3, wherein computing the Tm comprises identifying an inflection point in a signal intensity generated by each of the plurality of mismatch-tolerant probes in each partition.

5. The method according to claim 3, wherein d) further comprises classifying the Tm signature as belonging to a group of nucleic acid molecules.

6. The method according to claim 3, wherein d) further comprises counting the number of partitions with the same Tm signature.

7. The method according to claim 6, wherein d) further comprises determining a proportion of different groups of nucleic acid molecules in the sample based on the count numbers.

8. The method according to claim 1, wherein the amplification reaction comprises an asymmetric polymerase chain reaction (PCR).

9. The method according to claim 8, wherein the asymmetric PCR uses a primer set comprising:
a pair of forward and reverse primers for amplifying the nucleic acid molecules or parts thereof to produce double-stranded PCR products; and
a third primer for amplifying one of the two strands of the double-stranded PCR products or a part thereof.

10. The method according to claim 9, wherein the forward and reverse primers have a higher annealing temperature than the third primer.

11. The method according to claim 9, wherein the third primer is present at a higher concentration than the forward and reverse primers.

12. The method according to claim 1, wherein the plurality of mismatch-tolerant probes is configured to bind to a 16s and/or 18s ribosomal region of nucleic acid region of the microbiotic composition at temperatures below their melting temperature.

13. The method according to claim 1, wherein the plurality of mismatch-tolerant probes is configured to bind to a V3 region of a 16s and/or 18s ribosomal nucleic acid region of the nucleic acid molecules at temperatures below their melting temperatures.

14. The method according to claim 1, wherein the plurality of mismatch-tolerant probes is configured to have different Tm signatures for different groups of nucleic acid molecules, optionally
the plurality of mismatch-tolerant probes is configured to have different Tm signatures for nucleic acid molecules from different phyla and/or species of bacteria and/or fungus.

15. The method according to claim 1, wherein the plurality of mismatch-tolerant probes has one or more of the following properties:
is capable of producing a fluorescent signal;
has a greater number of mismatches with the sequences of one group of nucleic acid molecules than another group of nucleic acid molecules;
comprises an oligonucleotide comprising a reporter moiety and a quencher moiety;
comprises a molecular beacon;
each is capable of producing a distinct colorimetric signal, optionally wherein the colorimetric signal is selected from the group consisting of: a green signal, an orange signal and a red signal.

16. The method according to claim 1, wherein the microbiotic composition comprises one or more microbes comprising bacteria, fungi, and/or combination thereof.

17. The method according to claim 1, wherein the microbiotic composition comprises microbes from one or more bacteria from the genus of Acetobacter, Acinetobacter, Actinomyces, Agrobacterium spp., Azorhizobium, Azotobacter, Anaplasma spp., Bacillus spp., Bacteroides spp., Bartonella spp., Bordetella spp., Borrelia, Brucella spp., Burkholderia spp., Calymmatobacterium, Campylobacter, Chlamydia spp., Chlamydophila spp., Clostridium spp., Corynebacterium spp., Coxiella, Ehrlichia, Enterobacter, Enterococcus spp., Escherichia, Francisella, Fusobacterium, Gardnerella, Haemophilus spp., Helicobacter, Klebsiella, Lactobacillus spp., Lactococcus, Legionella, Listeria, Methanobacterium extroquens, Microbacterium multiforme, Micrococcus luteus, Moraxella catarrhalis, Mycobacterium spp., Mycoplasma spp., Neisseria spp., Pasteurella spp., Peptostreptococcus, Porphyromonas, Pseudomonas, Rhizobium, Rickettsia spp., Rochalimaea spp., Rothia, Salmonella spp., Serratia, Shigella, Staphylococcus spp., Stenotrophomonas, Streptococcus spp., Treponema spp., Vibrio spp., Wolbachia, and Yersinia spp, and/or
one or more fungus from the genus Absidia, Ajellomyces, Arthroderma, Aspergillus, Blastomyces, Candida, Cladophialophora, Coccidioides, Cryptococcus, Cunninghamella, Epidermophyton, Exophiala, Filobasidiella, Fonsecaea, Fusarium, Geotrichum, Histoplasma, Hortaea, Issatschenkia, Madurella, Malassezia, Microsporum, Microsporidia, Mucor, Nectria, Paecilomyces, Paracoccidioides, Penicillium, Pichia, Pneumocystis, Pseudallescheria, Rhizopus, Rhodotorula, Scedosporium, Schizophyllum, Sporothrix, Trichophyton, and Trichosporon.

18. The method according to claim 1, wherein the microbiotic composition comprises microbes from
one or more bacteria from the group consisting of Acetobacter aurantius, Acinetobacter baumannii, Actinomyces Israelii, Agrobacterium radiobacter, Agrobacterium tumefaciens, Azorhizobium caulinodans, Azotobacter vinelandii, Anaplasma phagocytophilum, Anaplasma marginale, Bacillus anthracis, Bacillus brevis, Bacillus cereus, Bacillus fusiformis, Bacillus licheniformis, Bacillus megaterium, Bacillus mycoides, Bacillus stearothermophilus, Bacillus subtilis, Bacteroides fragilis, Bacteroides gingivalis, Bacteroides melaminogenicus (Prevotella melaminogenica), Bartonella henselae, Bartonella quintana, Bordetella bronchiseptica, Bordetella pertussis, Borrelia burgdorferi, Brucella abortus, Brucella melitensis, Brucella suis, Burkholderia mallei, Burkholderia pseudomallei, Burkholderia cepacia complex, Burkholderia cenocepacia, Calymmatobacterium granulomatis, Campylobacter coli, Campylobacter fetus, Campylobacter jejuni, Campylobacter pylori, Chlamydia trachomatis, Chlamydophila. (such as C. pneumoniae, Chlamydophila psittaci, Clostridium botulinum, Clostridium difficile, Clostridium perfringens, Clostridium tetani), Corynebacterium diphtheriae, Corynebacterium fusiforme, Coxiella bumetii, Ehrlichia chaffeensis, Enterobacter cloacae, Enterococcus avium, Enterococcus durans, Enterococcus faecalis, Enterococcus faecium, Enterococcus galllinarum, Enterobacter gergoviae (now known as Pluralibacter gergoviae), Enterococcus maloratus, Escherichia coli, Francisella tularensis, Fusobacterium nucleatum, Gardnerella vaginalis, Haemophilus ducreyi, Haemophilus influenzae, Haemophilus parainfluenzae, Haemophilus pertussis, Haemophilus vaginalis, Helicobacter pylori, Klebsiella pneumoniae, Lactobacillus acidophilus, Lactobacillus casei, Lactococcus lactis, Legionella pneumophila, Listeria monocytogenes, Methanobacterium extroquens, Microbacterium multiforme, Micrococcus luteus, Moraxella catarrhalis, Mycobacterium avium, Mycobacterium bovis, Mycobacterium diphtheriae, Mycobacterium intracellulare, Mycobacterium leprae, Mycobacterium lepraemurium, Mycobacterium phlei, Mycobacterium smegmatis, Mycobacterium tuberculosis, Mycoplasma fermentans, Mycoplasma genitalium, Mycoplasma hominis, Mycoplasma penetrans, Mycoplasma pneumoniae, Neisseria gonorrhoeae, Neisseria meningitidis, Pasteurella multocida, Pasteurella tularensis Peptostreptococcus, Porphyromonas gingivalis, Pseudomonas aeruginosa, Rhizobium Radiobacter, Rickettsia prowazekii, Rickettsia psittaci, Rickettsia quintana, Rickettsia rickettsii, Rickettsia trachomae, Rochalimaea henselae, Rochalimaea quintana, Rothia dentocariosa, Salmonella enteritidis, Salmonella typhi, Salmonella typhimurium, Serratia marcescens, Shigella dysenteriae, Staphylococcus aureus, Staphylococcus epidermidis, Stenotrophomonas maltophilia, Streptococcus agalactiae, Streptococcus. avium, Streptococcus bovis, Streptococcus cricetus, Streptococcus faceium, Streptococcus faecalis, Streptococcus ferus, Streptococcus gallinarum, Streptococcus lactis, Streptococcus mitior, Streptococcus mitis, Streptococcus mutans, Streptococcus oralis, Streptococcus pneumoniae, Streptococcus pyogenes, Streptococcus rattus, Streptococcus salivarius, Streptococcus sanguis, Streptococcus sobrinus, Treponema pallidum, Treponema denticola, Vibrio cholerae, Vibrio comma, Vibrio parahaemolyticus, Vibrio vulnificus, Wolbachia, Yersinia enterocolitica, Yersinia pestis and Yersinia pseudotuberculosis, and/or
one or more fungus from the group consisting of Absidia corymbifera, Ajellomyces capsulatus, Ajellomyces dermatitidis, Arthroderma benhamiae, Arthroderma fulvum, Arthroderma gypseum, Arthroderma incurvatum, Arthroderma otae and Arthroderma vanbreuseghemii, Aspergillus flavus, Aspergillus fumigatus and Aspergillus niger, Blastomyces dermatitidis, Candida albicans, Candida glabrata, Candida guilliermondii, Candida krusei, Candida parapsilosis, Candida tropicalis and Candida pelliculosa, Cladophialophora carrionii, Coccidioides immitis and Coccidioides posadasii, Cryptococcus neoformans, Cunninghamella Sp, Epidermophyton floccosum, Exophiala dermatitidis, Filobasidiella neoformans, Fonsecaea pedrosoi, Fusarium solani, Geotrichum candidum, Histoplasma capsulatum, Hortaea werneckii, Issatschenkia orientalis, Madurella grisae, Malassezia furfur, Malassezia globosa, Malassezia obtusa, Malassezia pachydermatis, Malassezia restricta, Malassezia slooffiae, Malassezia sympodialis, Microsporum canis, Microsporum fulvum, Microsporum gypseum, Microsporidia, Mucor circinelloides, Nectria haematococca, Paecilomyces variotii, Paracoccidioides brasiliensis, Penicillium marneffei, Pichia anomala, Pichia guilliermondii, Pneumocystis jiroveci, Pneumocystis carinii, Pseudallescheria boydii, Rhizopus oryzae, Rhodotorula rubra, Scedosporium apiospermum, Schizophyllum commune, Sporothnx schenckii, Trichophyton mentagrophytes, Trichophyton rubrum, Trichophyton verrucosum and Trichophyton violaceum, and Trichosporon asahii, Trichosporon cutaneum, Trichosporon inkin and Trichosporon mucoides, and/or
combinations of bacteria and/or fungus thereof.

19. The method according to claim 1, wherein the sample is a medical sample and/or a non-medical sample, optionally
wherein the non-medical sample is a sample of one or more selected from the group consisting of food industries, consumer products, agriculture, and laboratory, or wherein the medical sample is a biological sample.

20. A microbiotic composition profiling kit, comprising:
a reagent for detecting a nucleic acid molecules of a microbiotic composition,
wherein the reagent comprises a plurality of mismatch-tolerant probes configured to have different Tm signatures for different groups of nucleic acid molecules of the microbiotic composition.
